(19) United States
(12) Patent Application Publication
(10) Pub. No.: US 2026/0209212 A1
(43) Pub. Date: Jul. 23, 2026

(54) HETEROCYCLIC GLP-1 AGONISTS

(71) Applicant: Gasherbrum Bio, Inc., South San Francisco, CA (US)

(72) Inventors: Chunliang Lu, Shanghai (CN); Zhongmiao Xu, Shanghai (CN); Ding Xue, Shanghai (CN); Haizhen Zhang, Shanghai (CN); Qiong Zhang, Shanghai (CN)

(73) Assignee: Gasherbrum Bio, Inc., South San Francisco, CA (US)

(21) Appl. No.: 19/142,131

(22) PCT Filed: Dec. 21, 2023
(86) PCT No.: PCT/CN2023/140456

§ 371 Date: Jun. 20, 2025

(30) Foreign Application Priority Data
Dec. 22, 2022 (WO) PCT/CN2022/141073

(51) Int. Cl. C07D 405/14; A61K 31/436; A61K 31/4439; A61K 31/454; A61K 45/06; C07D 491/056

(52) U.S. Cl. CPC C07D 405/14; A61K 31/436; A61K 31/4439; A61K 31/454; A61K 45/06; C07D 491/056

(57) ABSTRACT

The present disclosure relates generally to GLP-1 agonists and pharmaceutical compositions comprising the same, as well as methods for treating a GLP-1 associated disease, disorder, or condition.

### CROSS REFERENCE TO RELATED APPLICATIONS

This application claims the benefit of International Patent Application Number PCT/CN2022/141073 filed on Dec. 22, 2022, the content of which is hereby incorporated herein in its entirety.

### FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

In one aspect, provided are compounds of Formula I:

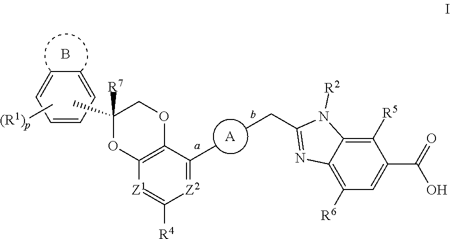

or a pharmaceutically acceptable salt, isotopically enriched analog, stereoisomer, mixture of stereoisomers, or prodrug thereof, wherein ring A, ring B, Z¹, Z², R¹, R², R⁴, R¹, R⁶, R⁷, and p are each independently as defined herein.

This disclosure also provides pharmaceutical compositions comprising one or more compounds of Formula I, or a pharmaceutically acceptable salt, isotopically enriched analog, stereoisomer, mixture of stereoisomers, or prodrug thereof, and a pharmaceutically acceptable excipient.

Also provided herein are pharmaceutical compositions comprising a compound of Formula I, or a pharmaceutically acceptable salt, isotopically enriched analog, stereoisomer, mixture of stereoisomers, or prodrug thereof, and a pharmaceutically acceptable excipient.

Also provided herein are methods for treating type 2 diabetes mellitus in a patient in need thereof, the methods comprising administering to the patient a therapeutically effective amount of a compound of Formula I, or a pharmaceutically acceptable salt, isotopically enriched analog, stereoisomer, mixture of stereoisomers, or prodrug thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I, or a pharmaceutically acceptable salt, isotopically enriched analog, stereoisomer, mixture of stereoisomers, or prodrug thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I, or a pharmaceutically acceptable salt, isotopically enriched analog, stereoisomer, mixture of stereoisomers, or prodrug thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I, or a pharmaceutically acceptable salt, isotopically enriched analog, stereoisomer, mixture of stereoisomers, or prodrug thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I, or a pharmaceutically acceptable salt, isotopically enriched analog, stereoisomer, mixture of stereoisomers, or prodrug thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I, or a pharmaceutically acceptable salt, isotopically enriched analog, stereoisomer, mixture of stereoisomers, or prodrug thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I, or a pharmaceutically acceptable salt, isotopically enriched analog, stereoisomer, mixture of stereoisomers, or prodrug thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I, or a pharmaceutically acceptable salt, isotopically enriched analog, stereoisomer, mixture of stereoisomers, or prodrug thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DESCRIPTION

Provided herein are heterocyclic GLP-1 agonists for use in the management of T2DM and other conditions where activation of GLP-1 activity is useful.

Before the present compounds and methods are described, it is to be understood that the disclosure is not limited to the methodologies, protocols, cell lines, assays, and reagents described, as these may vary. It is also to be understood that the terminology used herein is intended to describe embodiments of the present disclosure, and is in no way intended to limit the scope of the present disclosure as set forth in the appended claims.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%.

In certain other embodiments, the term “about” includes the indicated amount±1%. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₈ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃), and “propyl” includes n-propyl (i.e., —(CH₂)₂CH₃), and isopropyl (i.e., —CH(CH₃)₂).

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl, and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Thioalkoxy” refers to the group “alkyl-S—”.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6, or 1 to 3) hydrogen atoms are replaced by a halogen.

“Hydroxyalkyl” refers to an alkyl group as defined above, wherein one or more (e.g., 1 to 6, or 1 to 3) hydrogen atoms are replaced by a hydroxy group.

“Cyanoalkyl” refers to an alkyl group as defined above, wherein one, or one or more (e.g., 1 to 6, or 1 to 3) hydrogen atoms are replaced by a hydroxy group.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like.

Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroalkylene” refers to a divalent heteroalkyl group. “Heteroalkylene” groups must have at least one carbon and at least one heteroatomic group within the chain. The term “heteroalkylene” includes unbranched or branched saturated chain having carbon and heteroatoms. By way of example, 1, 2 or 3 carbon atoms may be independently replaced with the same or different heteroatomic group.

Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkylene groups include, e.g., —CH₂OCH₂—, —CH(CH₃)OCH₂—, —CH₂CH₂OCH₂—, —OCH₂—, —CH(CH₃)O—, —CH₂CH₂O—, —CH₂CH₂OCH₂CH₂OCH₂—, —CH₂CH₂OCH₂CH₂O—, —CH₂SCH₂—, —CH(CH₃)SCH₂—, —CH₂CH₂SCH₂—, —CH₂CH₂SCH₂CH₂SCH₂—, —SCH₂—, —CH(CH₃)S—, —CH₂CH₂S—, —CH₂CH₂SCH₂CH₂S—, —CH₂S(O)₂CH₂—, —CH(CH₃)S(O)₂CH₂—, —CH₂CH₂S(O)₂CH₂—, —CH₂CH₂S(O)₂CH₂CH₂OCH₂—, —CH₂NR^{y}CH₂—, —CH(CH₃)NR^{y}CH₂—, —CH₂CH₂NR^{y}CH₂—, —CH₂CH₂NR^{y}CH₂CH₂NR^{y}CH₂—, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkylene includes 1 to 10 carbon atoms, 1 to 8 carbon atoms, or 1 to 4 carbon atoms; and 1 to 3 heteroatoms, 1 to 2 heteroatoms, or 1 heteroatom.

As used herein, the term “heteroalkylene” does not include groups such as amides or other functional groups having an oxo present on one or more carbon atoms.

“Heteroaryl” refers to an aromatic group having a single ring or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl, benzotriazolyl, benzo[4,6]imidazo[1,2-a]pyridyl, carbazolyl, cinnolinyl, dibenzofuranyl, dibenzothienyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thienyl, triazolyl, tetrazolyl, and triazinyl. Examples of the fused-heteroaryl rings include, but are not limited to, benzo[d]thiazolyl, quinolinyl, isoquinolinyl, benzo[b]thienyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (=O) or N-oxide (—O—) moieties. Any non-aromatic ring or fused ring system containing at least one heteroatom and one non-aromatic ring is considered a heterocyclyl, regardless of the attachment to the remainder of the molecule. For example, fused ring systems such as decahydroquinazolinyl, 1,2,3,4-tetrahydroquinazolinyl, and 5,6,7,8-tetrahydroquinazolinyl are heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Sulfonyl” refers to the group —S(O)₂R³, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Alkylsulfonyl” refers to the group —S(O)₂R, where R is alkyl.

“Alkylsulfinyl” refers to the group —S(O)R, where R is alkyl.

The terms “optional” or “optionally” means that the subsequently described event or circumstance may or may not occur, and that the description includes instances where said event or circumstance occurs and instances in which it does not. Also, the term “optionally substituted” refers to any one or more (e.g., 1 to 5, or 1 to 3) hydrogen atoms on the designated atom or group may or may not be replaced by a moiety other than hydrogen.

As used herein, the term “compound,” is meant to include any or all stereoisomers, geometric isomers, tautomers, and isotopically enriched analogs (e.g., deuterated analogs) of the structures depicted. Compounds herein identified by name or structure as one particular tautomeric form are intended to include other tautomeric forms unless otherwise specified.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ⁵S, ¹⁸F, ³⁶Cl, ¹²³I, and ¹²⁵I, respectively. Various isotopically labeled compounds of the present disclosure, for example those into which radioactive isotopes such as 3H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. In some embodiments, the one or more substituents include, but are not limited to, alkyl, alkenyl, alkynyl, alkoxy, alkoxyalkyl, acyl, amino, amido, amidino, aryl, azido, carbamoyl, carboxyl, carboxyl ester, cyano, cycloalkyl, cycloalkylalkyl, guanidino, halo, haloalkyl, hydroxyalkyl, haloalkoxy, haloalkoxyalkyl, heteroalkyl, heteroaryl, heterocyclyl, hydroxy, hydrazino, imino, imido, oxo, nitro, sulfinyl, sulfonic acid, sulfonyl, thiocyanate, thiol, thione, or combinations thereof.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

As used herein, when a ring is described as being “aromatic,” it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thiazole, isoxazole, isothiazole, and the like. When a ring system comprising at least two rings is described as “aromatic,” it means the ring system comprises one or more aromatic ring(s). Accordingly, when a ring system comprising at least two rings is described as “non-aromatic,” none of the constituent rings of the ring system is aromatic.

As used herein, when a ring is described as being “partially unsaturated,” it means the ring has one or more additional degrees of unsaturation (in addition to the degree of unsaturation attributed to the ring itself; e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated,” it means the ring system comprises one or more partially unsaturated ring(s), provided that none of the constituent rings of the ring system is aromatic.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “GLP-1 R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (Tanzeum®), dulaglutide (LY2189265, Trulicity®), efpeglenatide, exenatide (Byetta®, Bydureon®, Exendin-4), liraglutide (Victoza®, NN2211), lixisenatide (Lyxumia®), semaglutide (Ozempic®), tirzepatide, ZP2929, NNC0113-0987, BPI-3016, and TT401.

The term “pharmaceutically acceptable” as used herein indicates that the compound, or salt or composition thereof is compatible chemically and/or toxicologically with the other ingredients comprising a formulation and/or the subject being treated therewith.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a subformula thereof, or a pharmaceutically acceptable salt or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt or solvate thereof as provided herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing,” as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the subject is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulate,” “modulating,” or “modulation,” as used herein, refers to a regulation or an adjustment (e.g., increase or decrease) and can include, for example agonism, partial agonism or antagonism.

### Compounds

Provided herein are compounds of Formula I:

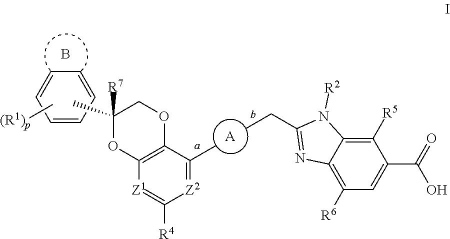

or a pharmaceutically acceptable salt, isotopically enriched analog, stereoisomer, mixture of stereoisomers, or prodrug thereof, wherein:
ring A is

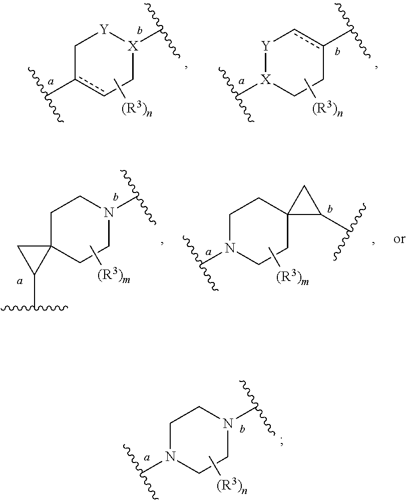

where [symbol] the bond represents a single or double bond;
where X is N or CH and Y is O, CH₂, CHR³, or C(R³)₂;
Z¹ is N or CR⁴;
Z² is N or CR⁴;
ring B is absent; or ring B is present and, together with the carbon atoms of the phenyl ring to which it is fused, forms a C₅₋₁₀ cycloalkyl, heterocyclyl, aryl, or heteroaryl; wherein ring B is independently optionally substituted with one to five R¹;
p is 0, 1, 2, or 3;
n is 0, 1, 2, or 3;
m is 0, 1, 2, or 3;
each R¹ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹OC(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₁₋₉ haloalkyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z;
R² is hydrogen, heterocyclyl, C₁₋₆ alkyl, or C₁₋₆ alkyl substituted with C₁₋₆ alkoxy, C₁₋₆ thioalkoxy, C₁₋₆ haloalkoxy, S(O)₂(C₁₋₆ alkyl), C₃₋₆cycloalkyl, 3- to 6-membered heterocyclyl, phenyl, or 5- to 6-membered heteroaryl; wherein each of the C₃₋₆ cycloalkyl, 3- to 6-membered heterocyclyl, phenyl, or 5- to 6-membered heteroaryl is optionally substituted with one to four R¹²;
R³ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀ cycloalkyl; or
two R³, together with the carbon atom to which they are attached, form a C₃₋₁₀ cycloalkyl or heterocyclyl; wherein each C₃₋₁₀cycloalkyl or heterocyclyl is independently optionally substituted with one or two halo;
each R⁴ is independently hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀cycloalkyl;
R¹ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁶ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁷ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀cycloalkyl;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z^{1a};
each R¹² is independently selected from the group consisting of cyano, halo, hydroxy, SH, nitro, C₁₋₆ alkyl, C₂₋₆ alkenyl, C₂₋₆ alkynyl, C₂₋₆ haloalkyl, C₁₋₆ cyanoalkyl, C₁₋₆ hydroxyalkyl, C₁₋₆ alkoxy, C₁₋₆ haloalkoxy, C₃₋₆ cycloalkyl, amino, C₁₋₆ alkylamino, and di-C₁₋₆ alkylamino;
each Z is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z^{1a};
each L¹ is independently —O—, —S—, —NR²⁰—, —C(O)—, —C(O)O—, —OC(O)—, —OC(O)O—, —C(O)NR²O, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a}; and
each Z^{1a} is independently halo, hydroxy, cyano, nitro, oxo, —SH, —NH₂, —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆cycloalkyl, heterocyclyl, aryl, or heteroaryl; wherein each —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆cycloalkyl, heterocyclyl, aryl, or heteroaryl of Z^{1a} is independently optionally substituted with one to five substituents selected from C₁₋₉ alkyl, oxo, halo, hydroxy, and cyano.

Provided herein are compounds of Formula I′:

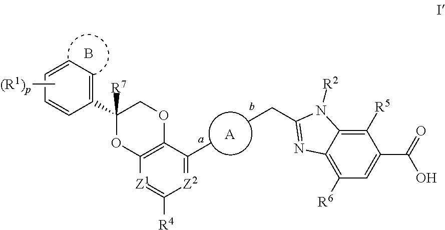

or a pharmaceutically acceptable salt, isotopically enriched analog, stereoisomer, mixture of stereoisomers, or prodrug thereof, wherein:
ring A is

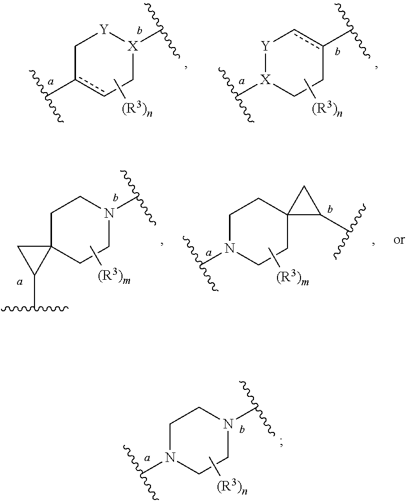

where [symbol] the bond represents a single or double bond;
where X is N or CH and Y is O, CH₂, CHR³, or C(R³)₂;
Z is N or CR⁴;
Z² is N or CR⁴;
ring B is absent; or ring B is present and, together with the carbon atoms of the phenyl ring to which it is fused, forms a C₅₋₁₀ cycloalkyl, heterocyclyl, aryl, or heteroaryl; wherein ring B is independently optionally substituted with one to five R¹;
p is 0, 1, 2, or 3;
n is 0, 1, 2, or 3;
m is 0, 1, 2, or 3;
each R¹ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹OC(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₁₋₉ haloalkyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z;
R² is hydrogen, heterocyclyl, C₁₋₆ alkyl, or C₁₋₆ alkyl substituted with C₁₋₆ alkoxy, C₁₋₆ thioalkoxy, C₁₋₆ (haloalkoxy, S(O)₂(C₁₋₆ alkyl), C₃₋₆ cycloalkyl, 3- to 6-membered heterocyclyl, phenyl, or 5- to 6-membered heteroaryl; wherein each of the C₃₋₆ cycloalkyl, 3- to 6-membered heterocyclyl, phenyl, or 5- to 6-membered heteroaryl is optionally substituted with one to four R¹²;
R³ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀ cycloalkyl; or
two R³, together with the carbon atom to which they are attached, form a C₃₋₁₀ cycloalkyl or heterocyclyl; wherein each C₃₋₁₀ cycloalkyl or heterocyclyl is independently optionally substituted with one or two halo;
each R⁴ is independently hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁵ is hydrogen, cyano, halo, hydroxy, C₁₋₆ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁶ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₆ alkoxy, C₁₋₆ haloalkyl, C₁₋₆ haloalkoxy, or C₃₋₁₀ cycloalkyl;
R⁷ is hydrogen, cyano, halo, hydroxy, C₁₋₆ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z^{1a};
each R¹² is independently selected from the group consisting of cyano, halo, hydroxy, SH, nitro, C₁₋₆ alkyl, C₂₋₆ alkenyl, C₂₋₆ alkynyl, C₁₋₆ (haloalkyl, C₁₋₆ cyanoalkyl, C₁₋₆ hydroxyalkyl, C₁₋₆ alkoxy, C₁₋₆ haloalkoxy, C₃₋₆ cycloalkyl, amino, C₁₋₆ alkylamino, and di-C₁₋₆ alkylamino;
each Z is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z^{1a};
each L¹ is independently —O—, —S—, —NR²⁰—, —C(O)—, —C(O)O—, —OC(O)—, —OC(O)O—, —C(O)NR²⁰, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Zia; and
each Z^{1a} is independently halo, hydroxy, cyano, nitro, oxo, —SH, —NH₂, —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆cycloalkyl, heterocyclyl, aryl, or heteroaryl; wherein each —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano.

In certain embodiments, one or more of the following occurs:
A) ring A is

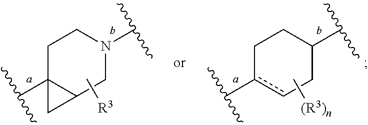

B) one of R¹ and R⁶ is other than hydrogen;
C) ring B is present;
D) at least one R⁴ is other than hydrogen;
E) R² is heterocyclyl optionally substituted with one to four R¹²; or
F) at least one R¹² is —C(O)—C₁₋₆ alkyl.

In certain embodiments, one or more of the following occurs:
A) ring A is

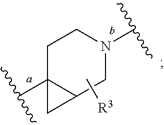

B) one of R¹ and R⁶ is other than hydrogen;
C) R² is heterocyclyl optionally substituted with one to four R¹²; or
D) at least one R¹² is —C(O)—C₁₋₆ alkyl.

In certain embodiments, ring A is:

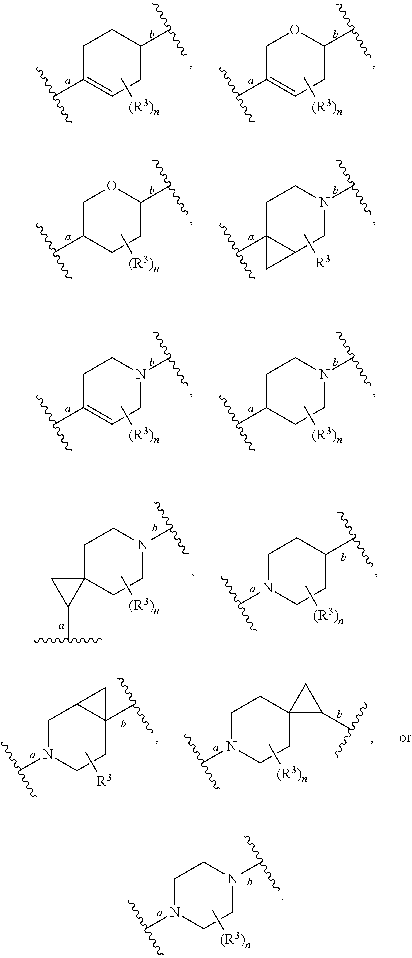

In certain embodiments, ring A is

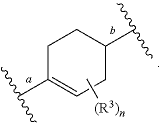

In certain embodiments, ring A is

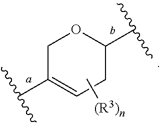

In certain embodiments, ring A is

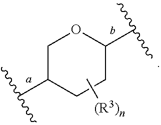

In certain embodiments, ring A is

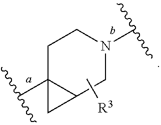

In certain embodiments, ring A is

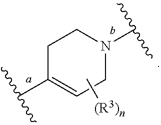

In certain embodiments, ring A is

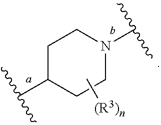

In certain embodiments, ring A is

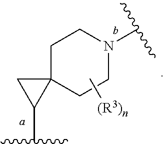

In certain embodiments, ring A is

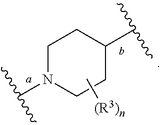

In certain embodiments, ring A is

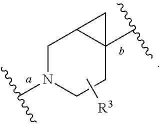

In certain embodiments, ring A is

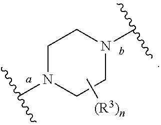

In certain embodiments, ring A is

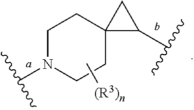

In certain embodiments, ring A is

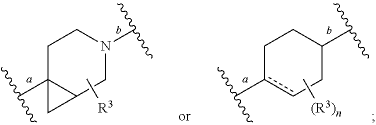

In certain embodiments, one of R⁵ and R⁶ is other than hydrogen.

In certain embodiments, ring B is present.

In certain embodiments, at least one R⁴ is other than hydrogen.

In certain embodiments, Z¹ is CR⁴; and R⁴ is other than hydrogen. In certain embodiments Z¹ is CR⁴; and R⁴ is other than hydrogen. In certain embodiments, Z² is CR⁴; and R⁴ is other than hydrogen

In certain embodiments, R² is heterocyclyl optionally substituted with one to four R¹².

In certain embodiments, at least one R¹² is —C(O)—C₁₋₆ alkyl.

In certain embodiments, n is 0. In certain embodiments, n is 1. In certain embodiments, n is 2.

In certain embodiments, in is 0.

In certain embodiments, each R³ is independently C₁₋₉ alkyl.

In certain embodiments, n is 1; and R³ is methyl.

In certain embodiments, two R³, together with the carbon atom to which they are attached, form a C₃₋₁₀ cycloalkyl or heterocyclyl; wherein each C₃₋₁₀ cycloalkyl or heterocyclyl is independently optionally substituted with one or two halo. In certain embodiments, two R³, together with the carbon atom to which they are attached, form a C₃₋₁₀ cycloalkyl optionally substituted with one or two halo. In certain embodiments, two R³, together with the carbon atom to which they are attached, form a C₃₋₁₀ cycloalkyl.

In certain embodiments, provided herein is a compound of Formula IA:

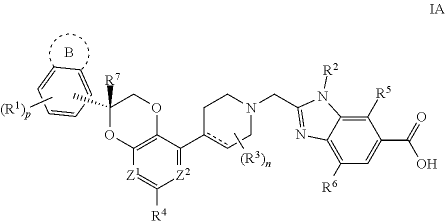

or a pharmaceutically acceptable salt, isotopically enriched analog, stereoisomer, mixture of stereoisomers, or prodrug thereof; wherein the dashed line, ring B, Z¹, Z², R¹, R², R³, R⁴, R⁵, R⁶, n, and p are each independently as defined herein.

In certain embodiments, provided herein is a compound of Formula IB:

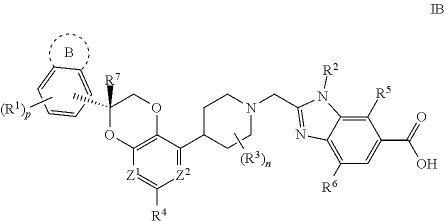

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula IC:

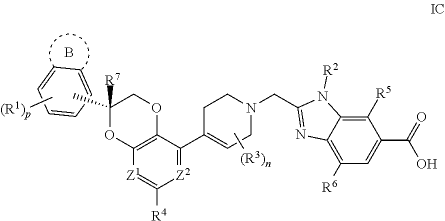

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula ID:

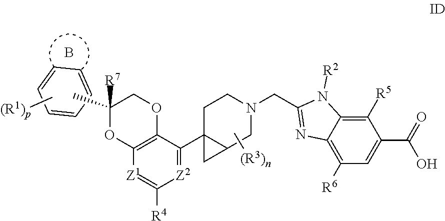

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, and p are each independently as defined herein.

In certain embodiments, provided herein is a compound of Formula IE:

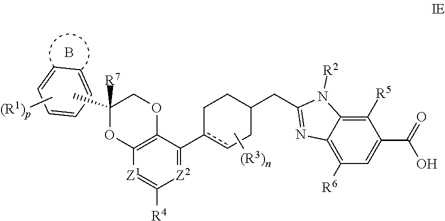

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula IF:

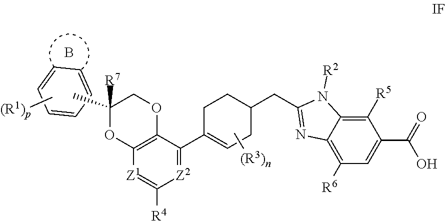

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula I′A:

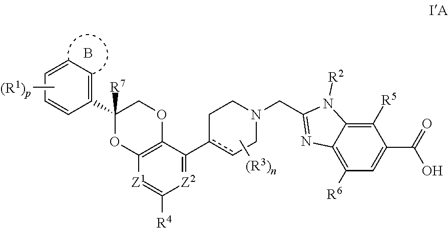

or a pharmaceutically acceptable salt, isotopically enriched analog, stereoisomer, mixture of stereoisomers, or prodrug thereof; wherein the dashed line, ring B, Z¹, Z², R¹, R², R³, R⁴, R⁵, R⁶, n, and p are each independently as defined herein.

In certain embodiments, provided herein is a compound of Formula I′B:

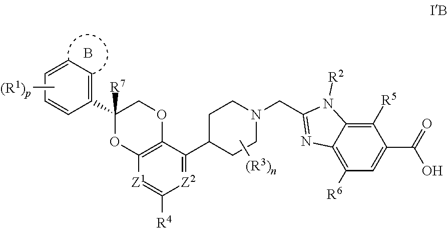

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula I′C:

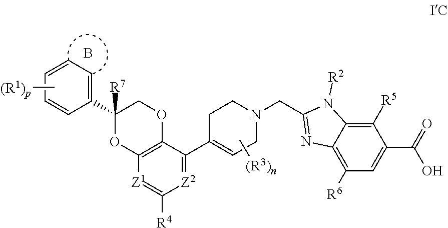

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula I′D:

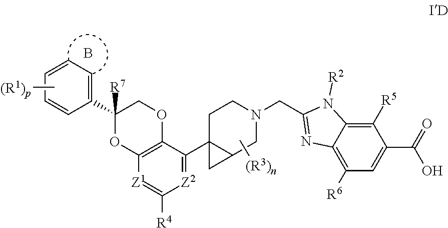

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, and p are each independently as defined herein.

In certain embodiments, provided herein is a compound of Formula I′E:

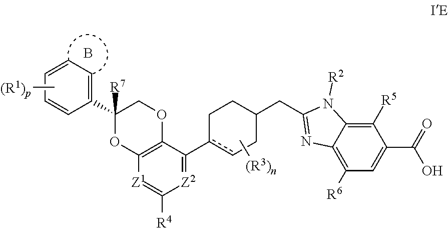

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In certain embodiments, provided herein is a compound of Formula I′F:

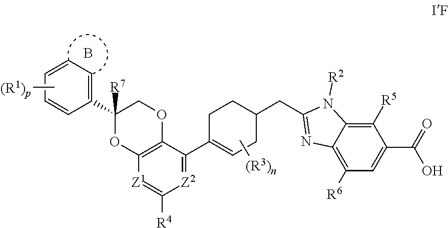

or a pharmaceutically acceptable salt, isotopically enriched analog, stereoisomer, mixture of stereoisomers, or prodrug thereof; wherein ring B, Z¹, Z², R¹, R², R³, R⁴, R⁵, R⁶, n, and p are each independently as defined herein.

In some embodiments, R⁵ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀ cycloalkyl.

In some embodiments, R⁶ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀ cycloalkyl.

In some embodiments, R⁵ is hydrogen, halo, or C₁₋₉ alkoxy. In some embodiments, R⁵ is hydrogen or halo. In some embodiments, R⁵ is hydrogen. In some embodiments, R⁵ is halo.

In some embodiments, R⁶ is hydrogen, halo, or C₁₋₉ alkoxy. In some embodiments, R⁶ is hydrogen. In some embodiments, R⁶ is halo. In some embodiments, R⁶ is C₁₋₉ alkoxy.

In some embodiments, at least one of R⁵ and R⁶ is halo or C₁₋₉ alkoxy.

In some embodiments, R⁵ is hydrogen or halo; and R⁶ is halo or C₁₋₉ alkoxy. In some embodiments, R⁵ is hydrogen; and R⁶ is halo or C₁₋₉ alkoxy. In some embodiments, R⁵ is hydrogen; and R⁶ is fluoro or methoxy.

In some embodiments, R⁵ is halo; and R⁶ is hydrogen, halo, or C₁₋₉ alkoxy. In some embodiments, R⁵ is halo; and R⁶ is hydrogen.

In some embodiments, ring B is absent. In some embodiments, ring B is present.

In some embodiments, ring B is present and, together with the carbon atoms of the phenyl ring to which it is fused, forms a C₅₋₁₀ cycloalkyl, heterocyclyl, aryl, or heteroaryl; wherein ring B is independently optionally substituted with one to five R¹.

In some embodiments, ring B is present and, together with the carbon atoms of the phenyl ring to which it is fused, forms a C₅₋₁₀ cycloalkyl optionally substituted with one to five R¹.

In some embodiments, ring B is present and, together with the carbon atoms of the phenyl ring to which it is fused, forms a heterocyclyl optionally substituted with one to five R¹.

In some embodiments, ring B is present and, together with the carbon atoms of the phenyl ring to which it is fused, forms an aryl optionally substituted with one to five R¹.

In some embodiments, ring B is present and, together with the carbon atoms of the phenyl ring to which it is fused, forms a heteroaryl optionally substituted with one to five R¹.

In some embodiments, the moiety

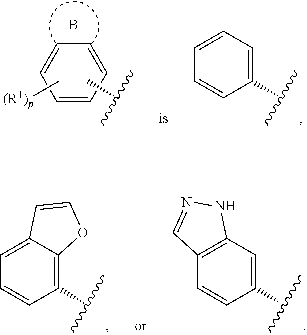

In some embodiments, the moiety

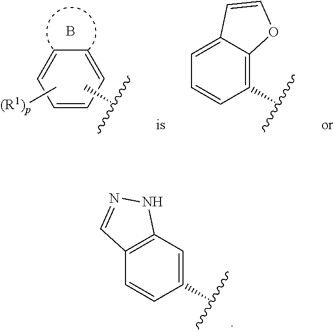

In some embodiments, the moiety

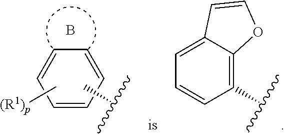

In some embodiments, the moiety

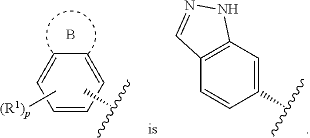

In some embodiments, the moiety

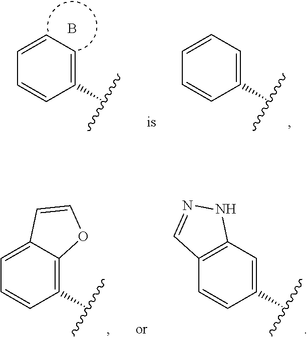

In some embodiments, the moiety

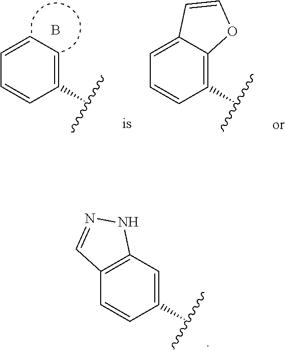

In some embodiments, the moiety

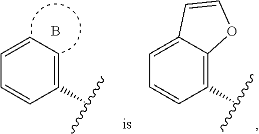

In some embodiments, the moiety

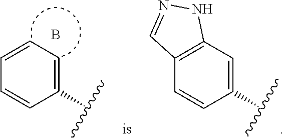

In some embodiments, each R¹ is independently halo, cyano, or C₁₋₉ haloalkyl.

In some embodiments, p is 1. In some embodiments, p is 1; and R¹ is halo, cyano, or C₁₋₉ haloalkyl.

In some embodiments, the moiety

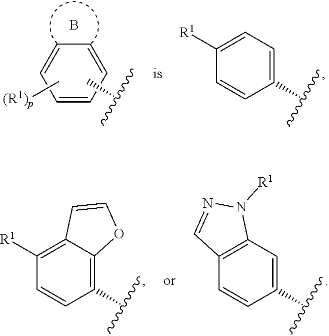

In some embodiments, the moiety

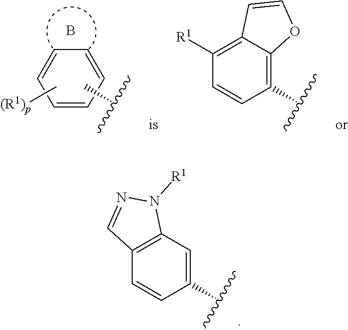

In some embodiments, the moiety

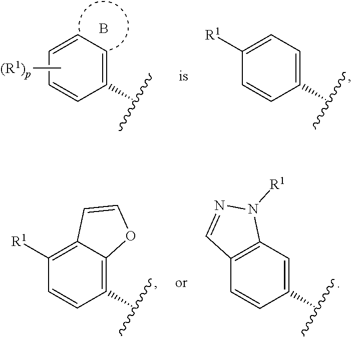

In some embodiments, the moiety

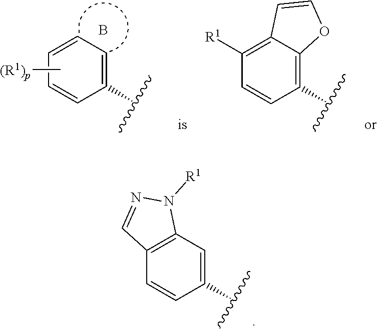

In some embodiments, R¹ is halo, cyano, or C₁₋₉ haloalkyl. In some embodiments, R¹ is halo or cyano. In some embodiments, R¹ is chloro or cyano.

In some embodiments, at least one R⁴ is other than hydrogen.

In some embodiments, at least one R⁴ is halo.

In some embodiments, Z² is CR⁴; and R⁴ of Z² is cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl.

In some embodiments, R² is heterocyclyl or C₁₋₆ alkyl substituted with C₁₋₆ (alkoxy, C₁₋₆ thioalkoxy, C₁₋₆ haloalkoxy, S(O)₂(C₁₋₆ alkyl), C₃₋₆ cycloalkyl, 3- to 6-membered heterocyclyl, phenyl, or 5- to 6-membered heteroaryl; wherein each of the heterocyclyl, C₃₋₆ cycloalkyl, 3- to 6-membered heterocyclyl, phenyl, or 5- to 6-membered heteroaryl is optionally substituted with one to four, or one to three, or one to two, or one R¹².

In some embodiments, R² is heterocyclyl or C₁₋₆ alkyl substituted with C₃₋₆ cycloalkyl, 3- to 6-membered heterocyclyl, or 5- to 6-membered heteroaryl; wherein each of the heterocyclyl, C₃₋₆ cycloalkyl, 3- to 6-membered heterocyclyl, or 5- to 6-membered heteroaryl is optionally substituted with one to four, or one to three, or one to two, or one R¹².

In some embodiments, R² is heterocyclyl or C₁₋₆ alkyl substituted with C₃₋₆ cycloalkyl, 3- to 6-membered heterocyclyl, or 5- to 6-membered heteroaryl; wherein each of the heterocyclyl, C₃₋₆ cycloalkyl, 3- to 6-membered heterocyclyl, or 5- to 6-membered heteroaryl is optionally substituted with one to four, or one to three, or one to two, or one R¹².

In some embodiments, R² is heterocyclyl or C₁₋₆ alkyl substituted with C₃₋₆cycloalkyl, 3- to 6-membered heterocyclyl, or 5- to 6-membered heteroaryl; wherein each of the heterocyclyl, C₃₋₆ cycloalkyl, 3- to 6-membered heterocyclyl, or 5- to 6-membered heteroaryl is optionally substituted with one to four, or one to three, or one to two, or one, cyano, halo, C₁₋₆ alkyl, C₁₋₆ cyanoalkyl, or —C(O)—C₁₋₆ alkyl.

In some embodiments, R² is heterocyclyl optionally substituted with one to four R¹².

In some embodiments, R² is 3-6 membered heterocyclyl substituted with C₁₋₆ (alkoxy.

R² is hydrogen, heterocyclyl, C₁₋₆ alkyl, or C₁₋₆ alkyl substituted with C₁₋₆ alkoxy, C₁₋₆ thioalkoxy, C₁₋₆ (haloalkoxy, S(O)₂(C₁₋₆ alkyl), C₃₋₆ cycloalkyl, 3- to 6-membered heterocyclyl, phenyl, or 5- to 6-membered heteroaryl; wherein each of the C₃₋₆ cycloalkyl, 3- to 6-membered heterocyclyl, phenyl, or 5- to 6-membered heteroaryl is optionally substituted with one to four R¹²;
each R¹² is independently selected from the group consisting of cyano, halo, hydroxy, SH, nitro, C₁₋₆ alkyl, C₂₋₆ alkenyl, C₂₋₆ alkynyl, C₁₋₆ haloalkyl, C₁₋₆ cyanoalkyl, C₁₋₆ hydroxyalkyl, C₁₋₆ alkoxy, C₁₋₆ haloalkoxy, —C(O)—C₁₋₆ alkyl, C₃₋₆ cycloalkyl, amino, C₁₋₆ alkylamino, and di-C₁₋₆ alkylamino;

In some embodiments, each R⁴ is independently hydrogen or halo.

In some embodiments, R⁷ is hydrogen.

In some embodiments, Z¹ is N.

In some embodiments, Z² is N.

In some embodiments, the moiety

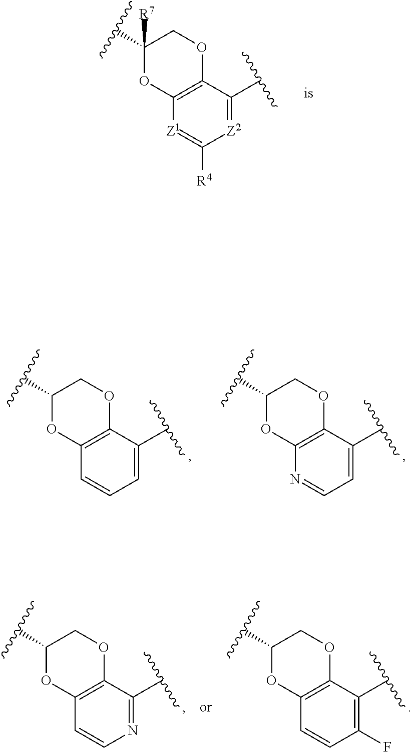

In certain embodiments, provided is a compound selected from Table 1, or a pharmaceutically acceptable salt, isotopically enriched analog, stereoisomer, mixture of stereoisomers, or prodrug thereof:

TABLE 1

| Compound | |
|---|---|
| No. | Structure |
| 101 | 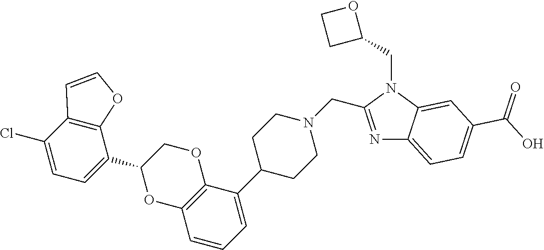 [SMILES: O=C(O)c1ccc2nc(CN3CCC(c4cccc5c4OC[C@@H](c4ccc(Cl)c6ccoc46)O5)CC3)n(C[C@@H]3CCO3)c2c1] |
| 102 | 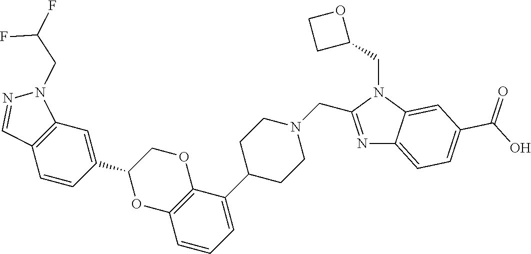 [SMILES: O=C(O)c1ccc2nc(CN3CCC(c4cccc5c4OC[C@@H](c4ccc6cnn(CC(F)F)c6c4)O5)CC3)n(C[C@@H]3CCO3)c2c1] |
| 103 | 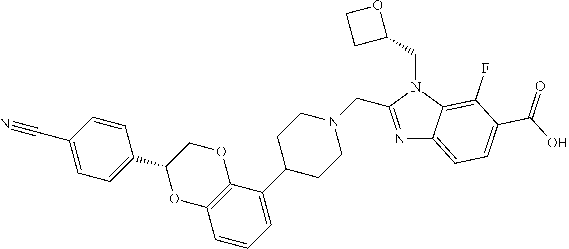 [SMILES: N#Cc1ccc([C@@H]2COc3c(cccc3C3CCN(Cc4nc5ccc(C(=O)O)c(F)c5n4C[C@@H]4CCO4)CC3)O2)cc1] |
| 104 | 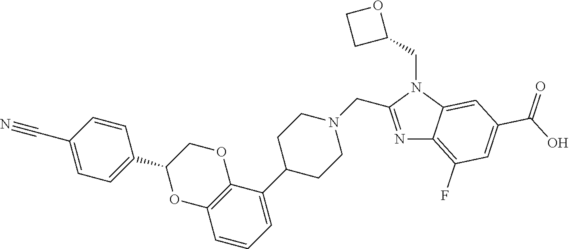 [SMILES: N#Cc1ccc([C@@H]2COc3c(cccc3C3CCN(Cc4nc5c(F)cc(C(=O)O)cc5n4C[C@@H]4CCO4)CC3)O2)cc1] |
| 105 | 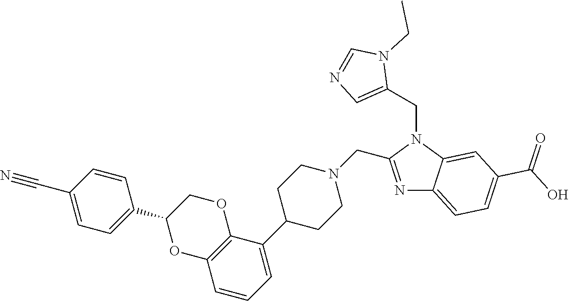 [SMILES: CCn1cncc1Cn1c(CN2CCC(c3cccc4c3OC[C@@H](c3ccc(C#N)cc3)O4)CC2)nc2ccc(C(=O)O)cc21] |
| 106 | 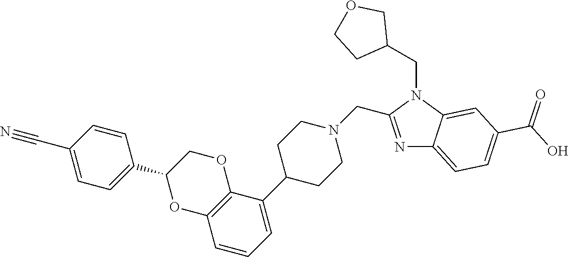 [SMILES: N#Cc1ccc([C@@H]2COc3c(cccc3C3CCN(Cc4nc5ccc(C(=O)O)cc5n4CC4CCOC4)CC3)O2)cc1] |
| 107 | 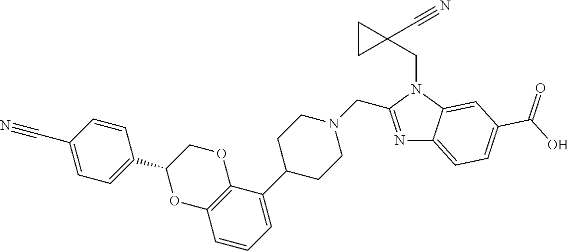 [SMILES: N#Cc1ccc([C@@H]2COc3c(cccc3C3CCN(Cc4nc5ccc(C(=O)O)cc5n4CC4(C#N)CC4)CC3)O2)cc1] |
| 108 | 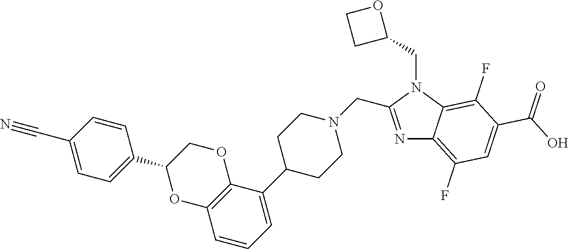 [SMILES: N#Cc1ccc([C@@H]2COc3c(cccc3C3CCN(Cc4nc5c(F)cc(C(=O)O)c(F)c5n4C[C@@H]4CCO4)CC3)O2)cc1] |
| 109 | 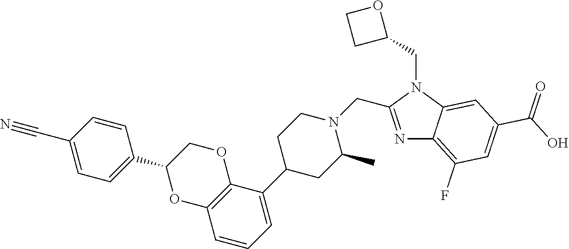 [SMILES: C[C@H]1CC(c2cccc3c2OC[C@@H](c2ccc(C#N)cc2)O3)CCN1Cc1nc2c(F)cc(C(=O)O)cc2n1C[C@@H]1CCO1] |
| 110 | 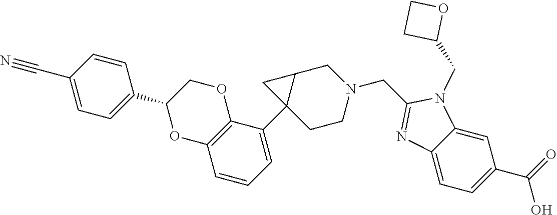 |
| 111 | 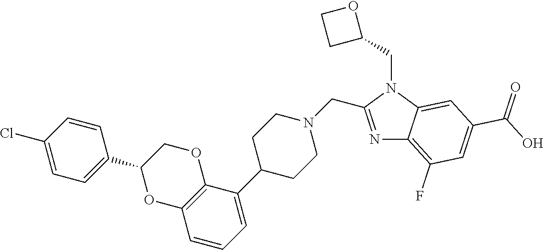 [SMILES: O=C(O)c1cc(F)c2nc(CN3CCC(c4cccc5c4OC[C@@H](c4ccc(Cl)cc4)O5)CC3)n(C[C@@H]3CCO3)c2c1] |
| 112 | 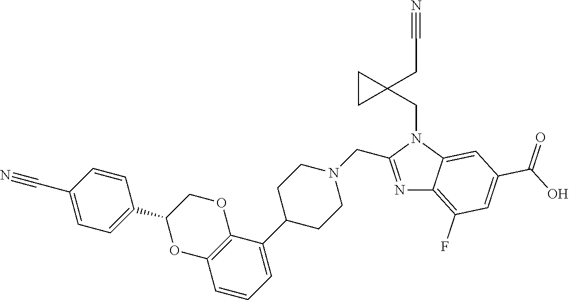 [SMILES: N#CCC1(Cn2c(CN3CCC(c4cccc5c4OC[C@@H](c4ccc(C#N)cc4)O5)CC3)nc3c(F)cc(C(=O)O)cc32)CC1] |
| 113 | 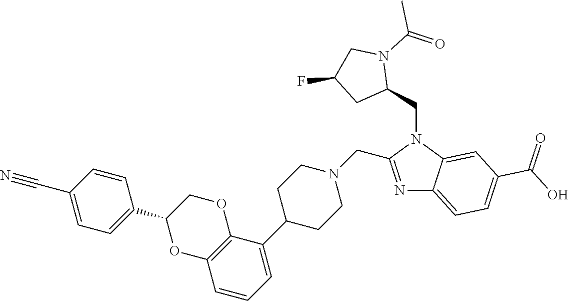 [SMILES: CC(=O)N1C[C@H](F)C[C@@H]1Cn1c(CN2CCC(c3cccc4c3OC[C@@H](c3ccc(C#N)cc3)O4)CC2)nc2ccc(C(=O)O)cc21] |
| 114 | 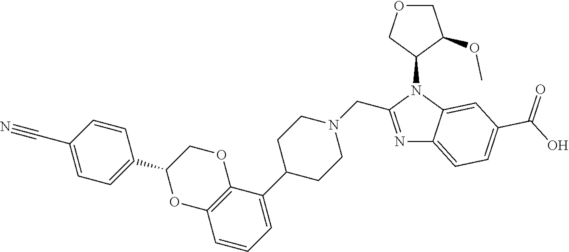 [SMILES: CO[C@@H]1COC[C@@H]1n1c(CN2CCC(c3cccc4c3OC[C@@H](c3ccc(C#N)cc3)O4)CC2)nc2ccc(C(=O)O)cc21] |
| 115 | 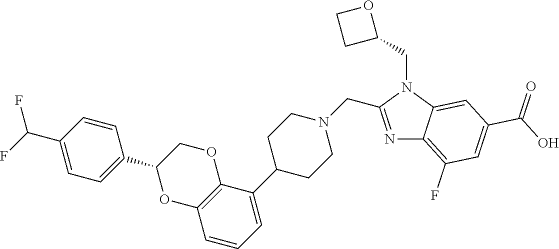 [SMILES: O=C(O)c1cc(F)c2nc(CN3CCC(c4cccc5c4OC[C@@H](c4ccc(C(F)F)cc4)O5)CC3)n(C[C@@H]3CCO3)c2c1] |
| 116 | 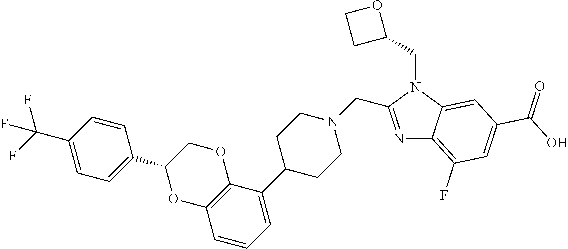 [SMILES: O=C(O)c1cc(F)c2nc(CN3CCC(c4cccc5c4OC[C@@H](c4ccc(C(F)(F)F)cc4)O5)CC3)n(C[C@@H]3CCO3)c2c1] |
| 117 | 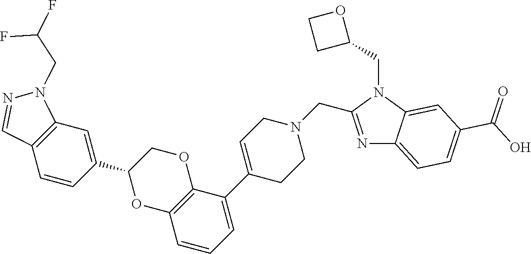 [SMILES: O=C(O)c1ccc2nc(CN3CC=C(c4cccc5c4OC[C@@H](c4ccc6cnn(CC(F)F)c6c4)O5)CC3)n(C[C@@H]3CCO3)c2c1] |
| 118 | 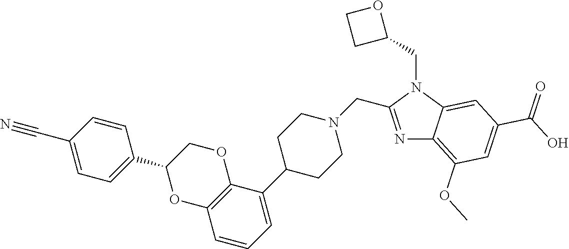 [SMILES: COc1cc(C(=O)O)cc2c1nc(CN1CCC(c3cccc4c3OC[C@@H](c3ccc(C#N)cc3)O4)CC1)n2C[C@@H]1CCO1] |
| 119 | 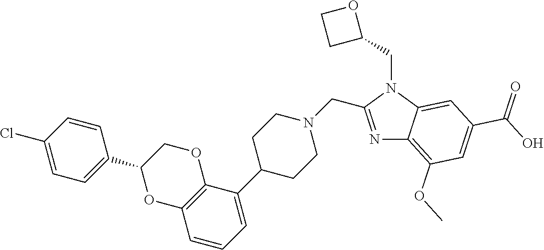 [SMILES: COc1cc(C(=O)O)cc2c1nc(CN1CCC(c3cccc4c3OC[C@@H](c3ccc(Cl)cc3)O4)CC1)n2C[C@@H]1CCO1] |
| 120 | 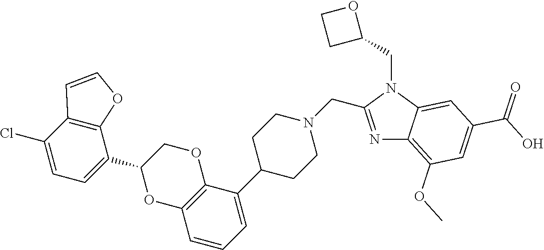 [SMILES: COc1cc(C(=O)O)cc2c1nc(CN1CCC(c3cccc4c3OC[C@@H](c3ccc(Cl)c5ccoc35)O4)CC1)n2C[C@@H]1CCO1] |
| 121 | 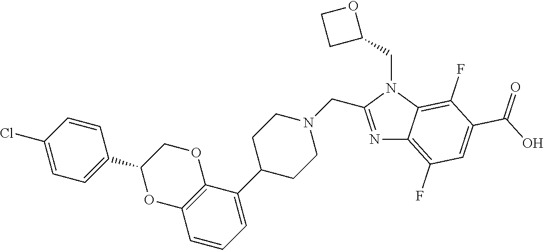 [SMILES: O=C(O)c1cc(F)c2nc(CN3CCC(c4cccc5c4OC[C@@H](c4ccc(Cl)cc4)O5)CC3)n(C[C@@H]3CCO3)c2c1F] |
| 122 | 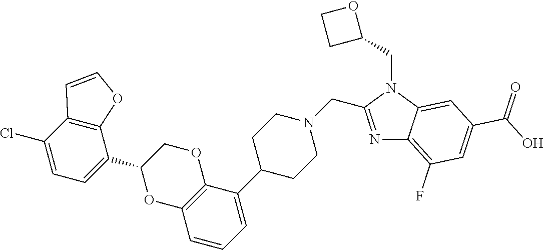 [SMILES: O=C(O)c1cc(F)c2nc(CN3CCC(c4cccc5c4OC[C@@H](c4ccc(Cl)c6ccoc46)O5)CC3)n(C[C@@H]3CCO3)c2c1] |
| 123 | 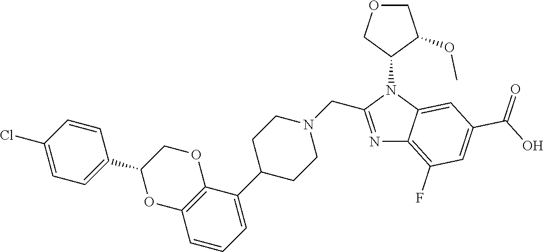 [SMILES: CO[C@H]1COC[C@H]1n1c(CN2CCC(c3cccc4c3OC[C@@H](c3ccc(Cl)cc3)O4)CC2)nc2c(F)cc(C(=O)O)cc21] |
| 124 | 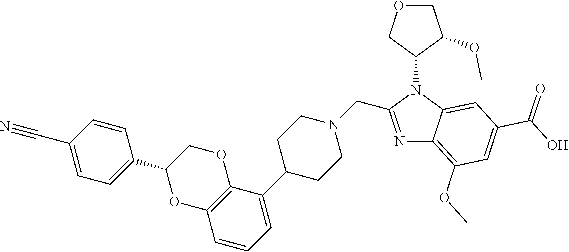 [SMILES: COc1cc(C(=O)O)cc2c1nc(CN1CCC(c3cccc4c3OC[C@@H](c3ccc(C#N)cc3)O4)CC1)n2[C@@H]1COC[C@@H]1OC] |
| 125 | 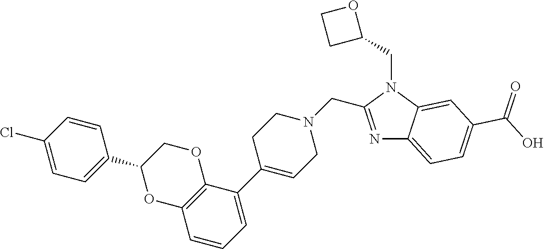 [SMILES: O=C(O)c1ccc2nc(CN3CC=C(c4cccc5c4OC[C@@H](c4ccc(Cl)cc4)O5)CC3)n(C[C@@H]3CCO3)c2c1] |
| 126 | 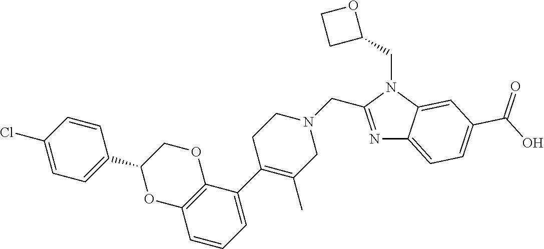 [SMILES: CC1=C(c2cccc3c2OC[C@@H](c2ccc(Cl)cc2)O3)CCN(Cc2nc3ccc(C(=O)O)cc3n2C[C@@H]2CCO2)C1] |
| 127 | 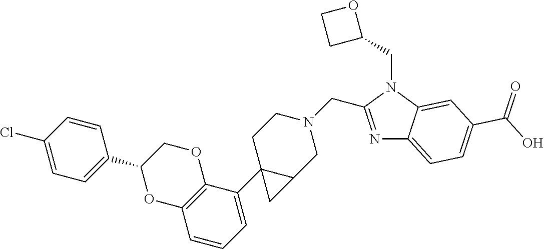 [SMILES: O=C(O)c1ccc2nc(CN3CCC4(c5cccc6c5OC[C@@H](c5ccc(Cl)cc5)O6)CC4C3)n(C[C@@H]3CCO3)c2c1] |
| 128 | 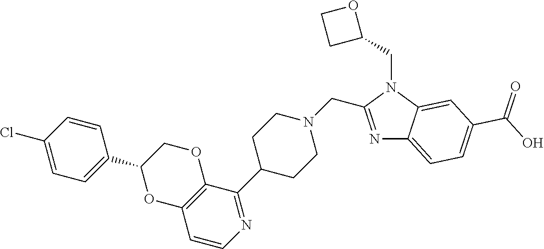 [SMILES: O=C(O)c1ccc2nc(CN3CCC(c4nccc5c4OC[C@@H](c4ccc(Cl)cc4)O5)CC3)n(C[C@@H]3CCO3)c2c1] |
| 129 | 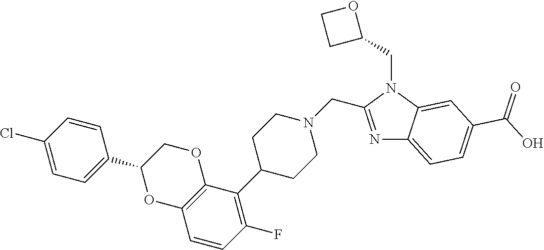 [SMILES: O=C(O)c1ccc2nc(CN3CCC(c4c(F)ccc5c4OC[C@@H](c4ccc(Cl)cc4)O5)CC3)n(C[C@@H]3CCO3)c2c1] |
| 130 | 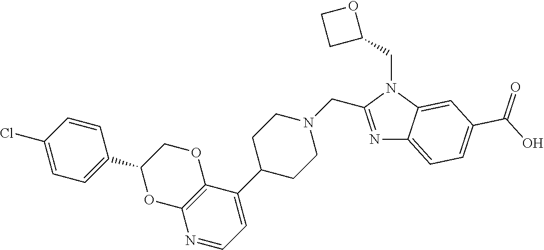 [SMILES: O=C(O)c1ccc2nc(CN3CCC(c4ccnc5c4OC[C@@H](c4ccc(Cl)cc4)O5)CC3)n(C[C@@H]3CCO3)c2c1] |

In certain embodiments, provided is a compound selected from compound 103, 104, 108, 109, 110, 111, 112, 113, 114, 115, 116, 118, 119, 120, 121, 122, 123, 124, and 127, or a pharmaceutically acceptable salt, isotopically enriched analog, stereoisomer, mixture of stereoisomers, or prodrug thereof.

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula I.

It will further be appreciated that the compounds of Formula I or their salts may be isolated in the form of solvates, and accordingly that any such solvate is included within the scope of the present disclosure. For example, compounds of Formula I and salts thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., a compound of Formula I, or a subformula thereof, or a pharmaceutically acceptable salt, isotopically enriched analog, stereoisomer, mixture of stereoisomers, or prodrug thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be necessary or desirable.

Also provided herein are pharmaceutical compositions which contain, as the active ingredient, a compound of Formula I, or a subformula thereof, or a pharmaceutically acceptable salt, isotopically enriched analog, stereoisomer, mixture of stereoisomers, or prodrug thereof, in combination with one or more pharmaceutically acceptable excipients (carriers). For example, a pharmaceutical composition prepared using a compound of Formula I, or a subformula thereof, or a pharmaceutically acceptable salt, isotopically enriched analog, stereoisomer, mixture of stereoisomers, or prodrug thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I, or a subformula thereof, or a pharmaceutically acceptable salt, isotopically enriched analog, stereoisomer, mixture of stereoisomers, or prodrug thereof with a pharmaceutically acceptable excipient. Pharmaceutical compositions containing a compound of Formula I, or a subformula thereof, or a pharmaceutically acceptable salt, isotopically enriched analog, stereoisomer, mixture of stereoisomers, or prodrug thereof as the active ingredient can be prepared by intimately mixing the compound of Formula I, or a subformula thereof, or a pharmaceutically acceptable salt, isotopically enriched analog, stereoisomer, mixture of stereoisomers, or prodrug thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, a compound of Formula I, or a subformula thereof, or a pharmaceutically acceptable salt, isotopically enriched analog, stereoisomer, mixture of stereoisomers, or prodrug thereof as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I, or a subformula thereof, or a pharmaceutically acceptable salt, isotopically enriched analog, stereoisomer, mixture of stereoisomers, or prodrug thereof, in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Formula I, or a subformula thereof, or a pharmaceutically acceptable salt, isotopically enriched analog, stereoisomer, mixture of stereoisomers, or prodrug thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I, or a subformula thereof, or a pharmaceutically acceptable salt, isotopically enriched analog, stereoisomer, mixture of stereoisomers, or prodrug thereof, as described herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, a compound of Formula I, or a subformula thereof, or a pharmaceutically acceptable salt, isotopically enriched analog, stereoisomer, mixture of stereoisomers, or prodrug thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I, or a subformula thereof, or a pharmaceutically acceptable salt, isotopically enriched analog, stereoisomer, mixture of stereoisomers, or prodrug thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I, or a subformula thereof, or a pharmaceutically acceptable salt, isotopically enriched analog, stereoisomer, mixture of stereoisomers, or prodrug thereof as described herein or a pharmaceutical composition thereof is formulated for ocular administration. In some embodiments, ocular compositions can include, without limitation, one or more of any of the following: viscogens (e.g., carboxymethylcellulose, glycerin, polyvinylpyrrolidone, polyethylene glycol); stabilizers (e.g., pluronic (triblock copolymers), cyclodextrins); preservatives (e.g., benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I, or a subformula thereof, or a pharmaceutically acceptable salt, isotopically enriched analog, stereoisomer, mixture of stereoisomers, or prodrug thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) (PLGA)-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet Formulation

The following ingredients are mixed intimately and pressed into single scored tablets.

| | Quantity per |
|---|---|
| Ingredient | tablet, mg |
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for a compound of Formula I, or a subformula thereof, or a pharmaceutically acceptable salt, isotopically enriched analog, stereoisomer, mixture of stereoisomers, or prodrug thereof, is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I, or a subformula thereof, or a pharmaceutically acceptable salt, isotopically enriched analog, stereoisomer, mixture of stereoisomers, or prodrug thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I, or a subformula thereof, or a pharmaceutically acceptable salt, isotopically enriched analog, stereoisomer, mixture of stereoisomers, or prodrug thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I, or a subformula thereof, or a pharmaceutically acceptable salt, isotopically enriched analog, stereoisomer, mixture of stereoisomers, or prodrug thereof as described herein is administered as a dosage of about 100 mg/Kg.

In some embodiments, the foregoing dosages of a compound of Formula I, or a subformula thereof, or a pharmaceutically acceptable salt, isotopically enriched analog, stereoisomer, mixture of stereoisomers, or prodrug thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I, or a subformula thereof, or a pharmaceutically acceptable salt, isotopically enriched analog, stereoisomer, mixture of stereoisomers, or prodrug thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I, or a subformula thereof, or a pharmaceutically acceptable salt, isotopically enriched analog, stereoisomer, mixture of stereoisomers, or prodrug thereof is administered to a patient for a period of time followed by a separate period of time where administration of the compound of Formula I, or a subformula thereof, or a pharmaceutically acceptable salt, isotopically enriched analog, stereoisomer, mixture of stereoisomers, or prodrug thereof is stopped. In some embodiments, a compound of Formula I, or a subformula thereof, or a pharmaceutically acceptable salt, isotopically enriched analog, stereoisomer, mixture of stereoisomers, or prodrug thereof is administered for a first period and a second period following the first period, with administration stopped during the second period, followed by a third period where administration of the compound of Formula I, or a subformula thereof, or a pharmaceutically acceptable salt, isotopically enriched analog, stereoisomer, mixture of stereoisomers, or prodrug thereof is started and then a fourth period following the third period where administration is stopped. For example, the period of administration of a compound of Formula I, or a subformula thereof, or a pharmaceutically acceptable salt, isotopically enriched analog, stereoisomer, mixture of stereoisomers, or prodrug thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I, or a subformula thereof, or a pharmaceutically acceptable salt, isotopically enriched analog, stereoisomer, mixture of stereoisomers, or prodrug thereof, is orally administered to the patient one or more times per day (e.g., one time per day, two times per day, three times per day, four times per day per day or a single daily dose).

In some embodiments, a compound of Formula I, or a subformula thereof, or a pharmaceutically acceptable salt, isotopically enriched analog, stereoisomer, mixture of stereoisomers, or prodrug thereof, is administered by parenteral administration to the patient one or more times per day (e.g., 1 to 4 times, one time per day, two times per day, three times per day, four times per day or a single daily dose).

In some embodiments, a compound of Formula I, or a subformula thereof, or a pharmaceutically acceptable salt, isotopically enriched analog, stereoisomer, mixture of stereoisomers, or prodrug thereof, is administered by parenteral administration to the patient weekly.

### Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein.

Also provided herein is a method of treating type 2 diabetes mellitus in a patient in need thereof, the method comprising administering to the patient a therapeutically effective amount of a compound of Formula I, or a subformula thereof, or a pharmaceutically acceptable salt, isotopically enriched analog, stereoisomer, mixture of stereoisomers, or prodrug thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, abetalipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver).

NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, Steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US2006/0275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US2012/0021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related Conditions and Disorders

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., US20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the condition, disease or disorder is an eating disorder, such as hyperphagia, binge eating, bulimia, or compulsive eating.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of Formula I, or a subformula thereof, or a pharmaceutically acceptable salt, isotopically enriched analog, stereoisomer, mixture of stereoisomers, or prodrug thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11p-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in W0006/112549, W0007/028135, W0008/047821, W0008/050821, W0008/136428 and W0008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), farnesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an acetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, Schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropyridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein IIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in W0007/013694, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), and NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). It is to be understood that when referring to a therapeutically effective amount of an anti-emetic agent, the amount administered is an amount needed to counteract (e.g., reduce or remove) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: granisetron (Kytril), dolasetron, ondansetron (Zofran), tropisetron, ramosetron, palonosetron, alosetron, azasetron, bemesetron, zatisetron, batanopirde, MDL-73147EF; metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, tropanyl-3,5-dimethylbenzoate, 3-(4-allylpiperazin-1-yl)-2-quinoxalinecarbonitrile maleate, zacopride hydrochloride, and mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+ netupitant), quetiapine, gamosetron, ramosteron, ricasetron, risperidone, ziprasidone, and zatosetron.

In certain embodiments, the 5HT-3-receptor antagonist is granisetron, dolasetron, ondansetron hydrochloride, tropisetron, ramosetron, palonosetron, alosetron, bemesetron, zatisetron, batanopirde, MDL-73147EF, metoclopramide, N-3389, Y-25130 hydrochloride, MDL 72222, tropanyl-3,5-dimethylbenzoate 3-(4-allyl-piperazin-1-yl)-2-quinoxalinecarbonitrile maleate, zacopride hydrochloride and mirtazepine.

In certain embodiments, the 5HT-3-receptor antagonist is granisetron, dolasetron, ondansetron hydrochloride, tropisetron, ramosetron, palonosetron, alosetron, bemesetron, and zatisetron.

In certain embodiments, the 5HT-3-receptor antagonist is granisetron, dolasetron and ondansetron.

In certain embodiments, the 5HT-3-receptor antagonist is granisetron.

In certain embodiments, the 5HT-3-receptor antagonist is ondansetron.

In some embodiments, the anti-emetic agent is an antihistamine. Non-limiting examples of antihistamines include: piperazine derivatives (e.g., cyclizine, meclizine, and cinnarizine); promethazine; dimenhydrinate (Dramamine, Gravol); diphenhydramine; hydroxyzine; buclizine; and meclizine hydrochloride (Bonine, Antivert), doxylamine, and mirtazapine.

In some embodiments, the anti-emetic agent is an anticholinergic agent (inhibitors of the acetylcholine receptors). Non-limiting examples of anticholinergic agents include: atropine, scopolamine, glycopyrron, hyoscine, Artane (trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, dexamethasone, methylprednisolone, Prednisone®, and trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH₂OCO]—(R)-alpha-MeTrp-(S)—NHCH(CH₃) Ph), FK888, and (D-Pro4, D-Trp7,9,10, Phel1)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine); benzamides (e.g., metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., cannabidiol (CBD), cannabidiol dimethylheptyl (CBD-DMH), tetra-hydro-cannabinol (THC), cannabinoid agonists such as WIN 55-212 (a CB1 and CB2 receptor agonist), dronabinol (Marinol®), and nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### General Synthetic Methods

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme I illustrates a general method which can be employed for the synthesis of compounds described herein, where ring A, ring B, Z¹, Z², R¹, R², R⁴, R⁵, R⁶, R⁷, and p are each independently as defined herein, LG is a leaving group, such as halo (e.g., Cl, Br, or I), and R is hydrogen or a suitable carboxyl protecting group, such as alkyl or benzyl.

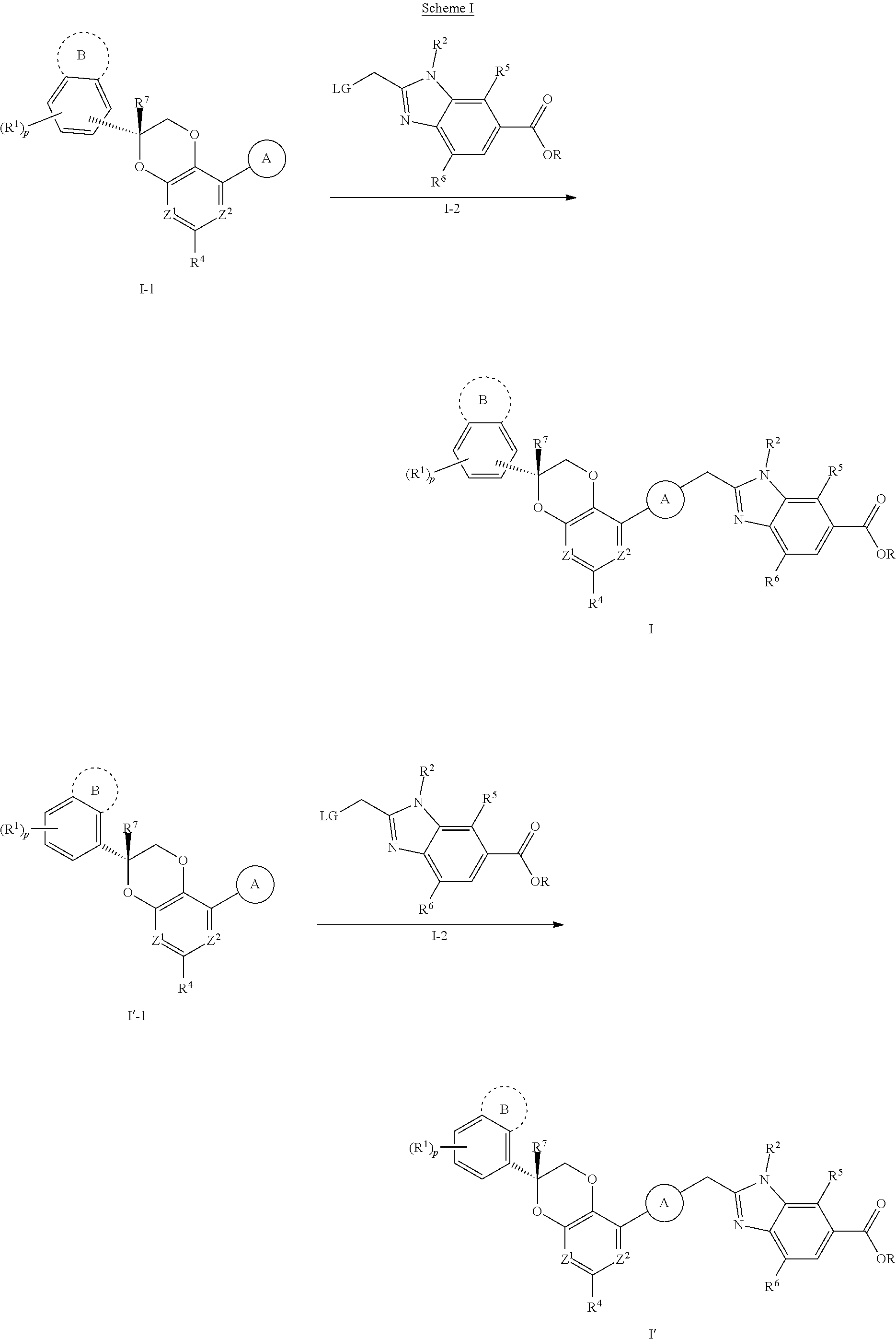

Compounds of Formula I and I′ can be provided by coupling compound I-1 or I′-1 with compound 1-2 under suitable coupling reaction conditions, such as SN2 reaction conditions, where ring A comprises a complimentary functional group such as, but not limited to, a nucleophilic moiety. Exemplary suitable reaction conditions include, but are not limited to, a polar aprotic solvent (e.g., acetonitrile), optionally in the presence of a base (e.g., an amine base, such as DIEA, or potassium carbonate, etc.). For processes where R is a carboxyl protecting group, such as alkyl or benzyl, the process may further comprise a deprotection step, e.g., a hydrolysis step. Suitable reagents for the deprotection step may comprise the presence of water under basic conditions, such as LiOH in water or a suitable aqueous solvent mixture (e.g., water in methanol, THF, or a combination thereof.

It should be appreciated that further derivatization can be performed of the resulting product via methods and chemical transformations which are known to those of skill in the art can provide alternative compounds of Formula I, I′, or other subformula thereof. For example, when the leaving group is an electrophile, such as an aldehyde, the coupling reaction conditions may comprise reductive amination reaction conditions. Thus, the conversion may comprise more than one reaction or set of reactants.

For any compound shown in Scheme I, it should be understood that various derivatives can be provided by functional group interconversion at any step. For example with R as alkyl, various compounds of Formula I, I′, or other subformula thereof can be provided via transesterification or hydrolysis using methods known to one of skill in the art. Likewise, various compounds of Formula I can be prepared by contacting compounds where one or more substituent (e.g., R¹, R², R³, R⁴, R⁵, R⁶, R⁷) is or comprises a leaving group (e.g., halo, such as Cl, Br, or I, or a pseudohalide, such as a triflate, sulfonate, or phosphate), under suitable coupling reaction conditions with a reagent comprising a complimentary functional group such as, but not limited to, a boronic acid or a derivative thereof, such as a boronic ester, zinc or magnesium halide, an organotin compound, such as tributylstannane or trimethylstannane, fluorosulfonyl esters, tin, sodium, hydrogen, and the like. Such reactions are commonly utilized for aromatic functionalization, and are typically conducted in the presence of suitable catalyst such as, but not limited to, a palladium catalyst including [1,1′-bis(diphenylphosphino)ferrocene]palladium(II) dichloride, Pd(OAc)₂, Pd(PPh₃)₄, PdCl₂(PPh₃)₂ or tris(dibenzylideneacetone)dipalladium(O), and the like, or a copper catalyst such as CuCl or CuI, and if required suitable mediator, co-catalyst and/or base known to one skilled in the art using suitable solvents/solvent mixtures. Upon reaction completion, compounds of Formula I can be recovered by conventional techniques such as neutralization, extraction, precipitation, chromatography, filtration and the like. In certain embodiments, when control of stereochemistry is desired, proper control of reaction conditions and selection of substituents for the reagents can at least partially dictate or preserve the formation of the various stereoisomers.

In some embodiments, the various substituents of Formula I-1, I′-1, or 1-2 are as defined herein. However, derivatization thereof prior to reacting in any step, and/or further derivatization of the resulting reaction product, provides various compounds of Formula I or I′. Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration, and the like. Other modifications to arrive at compounds of this disclosure are within the skill of the art.

In some embodiments, provided is a process for preparing a compound of Formula I, or a pharmaceutically acceptable salt, isotopically enriched analog, stereoisomer, mixture of stereoisomers, or prodrug thereof, comprising contacting a compound of Formula I-1:

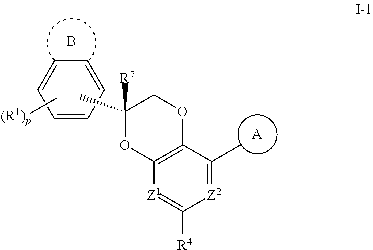

with a compound of Formula I-2:

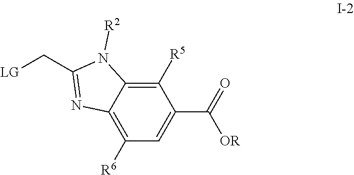

wherein ring A, ring B, Z¹, Z², R¹, R², R⁴, R¹, R⁶, R⁷, and p are each independently as defined herein; LG is a leaving group; and R is hydrogen or C₁₋₆ alkyl optionally substituted with phenyl, under conditions sufficient to provide the compound of Formula I, or a pharmaceutically acceptable salt, isotopically enriched analog, stereoisomer, mixture of stereoisomers, or prodrug thereof.

In some embodiments, provided is a process for preparing a compound of Formula I′, or a pharmaceutically acceptable salt, isotopically enriched analog, stereoisomer, mixture of stereoisomers, or prodrug thereof, comprising contacting a compound of Formula I′-1:

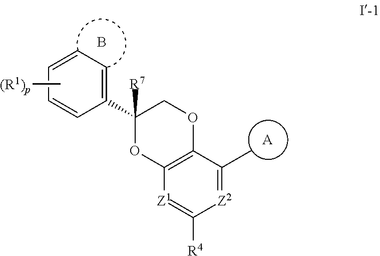

with a compound of Formula I-2:

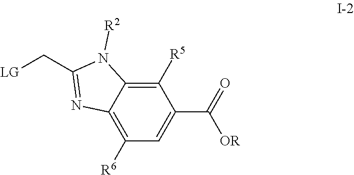

wherein ring A, ring B, Z¹, Z², R¹, R², R⁴, R¹, R⁶, R⁷, and p are each independently as defined herein; LG is a leaving group; and R is hydrogen or C₁₋₆ alkyl optionally substituted with phenyl, under conditions sufficient to provide the compound of Formula I′, or a pharmaceutically acceptable salt, isotopically enriched analog, stereoisomer, mixture of stereoisomers, or prodrug thereof.

In some embodiments, the process further comprises a hydrolysis or transesterification step prior to or after the contacting step. In some embodiments, the process comprises a base. In some embodiments, the process comprises elevated temperature.

### EXAMPLES

This disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Abbreviations (as Used Herein):

| | |
|---|---|
| AcOH | acetic acid |
| AcOK | potassium acetate |
| aq. | aqueous |
| CH₃CN | acetonitrile |
| Cs₂CO₃ | cesium carbonate |
| CuI | cuprous iodide |
| DCM or CH₂Cl₂ | dichloromethane |
| DIEA | N,N-Diisopropylethylamine |
| DMF | N,N-dimethyl-formamide |
| EtOAc | ethyl acetate |
| EtOH | ethanol |
| h | hour |
| HCl | hydrochloric acid |
| H₂O | water |
| prep. HPLC | Preparative High Performance |
| | Liquid Chromatography |
| prep. TLC | Preparative Thin Layer Chromatography |
| LiOH | lithium hydroxide |
| MeOH | methanol |
| NaBO₃ | sodium perborate |
| Na₂SO₄ | sodium sulfate |
| NaOH | sodium hydroxide |
| NBS | N-Bromosuccinimide |
| NH₄Cl | ammonium chloride |
| Pd(dppf)Cl₂ | [1,1′-bis(diphenylphosphino) |
| | ferrocene]dichloropalladium(II) |
| PE | petrol ether |
| PtO₂ | platinum oxide |
| RuCl[(S,S)- | chloro(p-cymene)[(S,S)-N-(p- |
| Tsdpen](p-cymene) | toluenesulfonyl)-1,2-diphenylethylene- |
| | diamine]ruthenium(II), CAS RN: 192139-90-5. |
| sat. | saturated |
| TEA | triethylamine |
| TFA | trifluoroacetic acid |
| THF | tetrahydrofuran |

General information: All evaporations or concentrations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H and ¹⁹F chemical shifts are reported in 6 values in ppm with the deuterated solvent as the internal standard. Data are reported as follows: chemical shift, multiplicity (s=singlet, d=doublet, t=triplet, q=quartet, br=broad, in =multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### General Synthetic Schemes

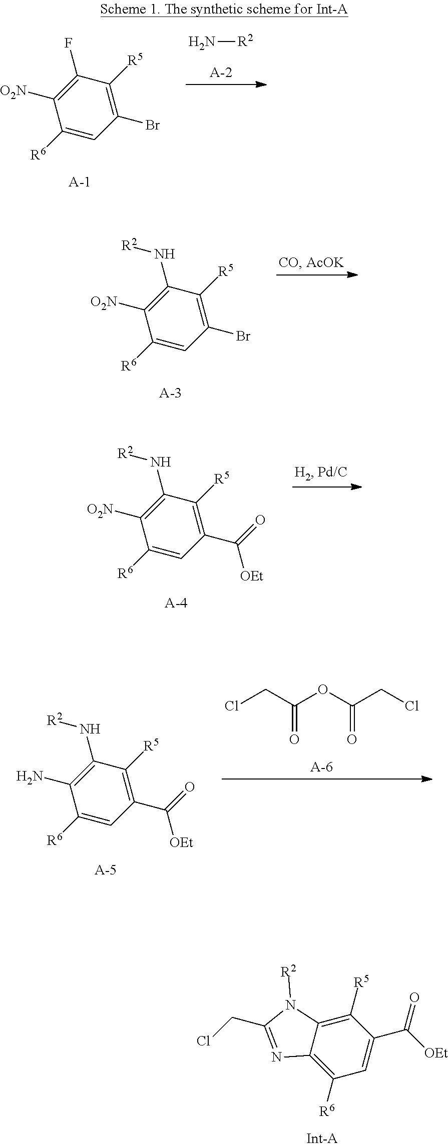

Key intermediate Int-A is synthesized through the method depicted in Scheme 1. A-1 goes through SNAr reaction with appropriate amine A-2 to give A-3. In certain embodiments, the reaction comprises DIEA. A-3 is then subjected to palladium-catalyzed carbonylative Suzuki coupling to generate intermediate A-4. In certain embodiments, the reaction comprises Pd(dppf)Cl₂. The nitro group of A-4 is then reduced by catalytic hydrogenation and the phenyl diamine obtained is reacted with 2-chloroacetic anhydride A-6, for example in dioxane, to give Int-A.

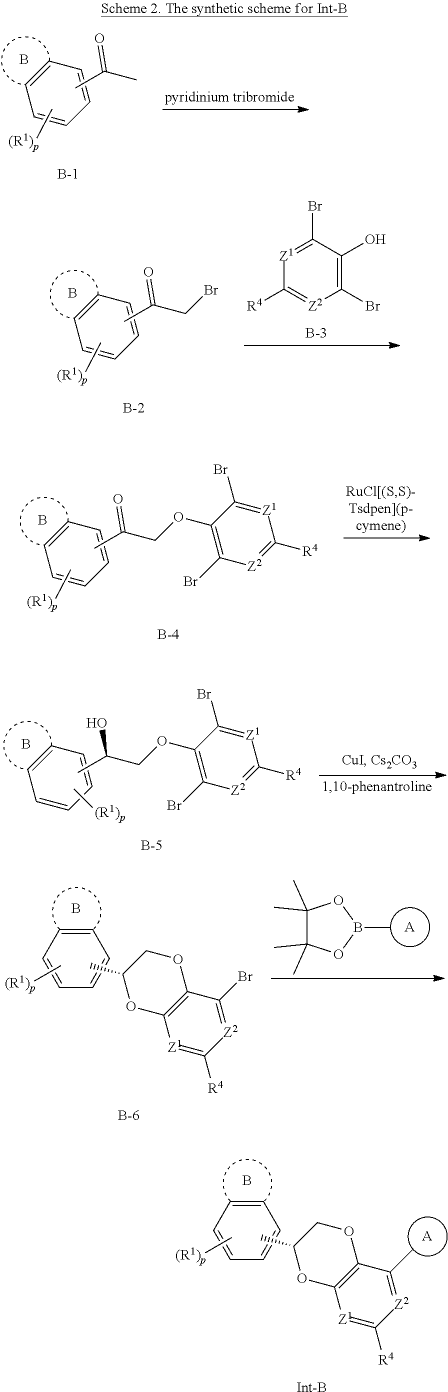

The synthesis of Int-B starts with bromination of acetophenone B-1, for example using pyridinium tribromide, to give B-2, which is then reacted with B-3 to give B-4. In certain embodiments, the reaction comprises a base, such as K₂CO₃. B-4 is then enantioselectively reduced by RuCl[(S,S)-Tsdpen](p-cymene) and goes through a intramolecular coupling reaction to give B-6. In certain embodiments, the reaction from B-4 to B-5 comprises TEA and formic acid. In certain embodiments, the reaction from B-5 to B-6 comprises toluene and heat. B-6 is then coupled with benzylboronic acid pinacol ester B-7 to give Int-B. In certain embodiments, the reaction comprises Pd(dppf)Cl₂.

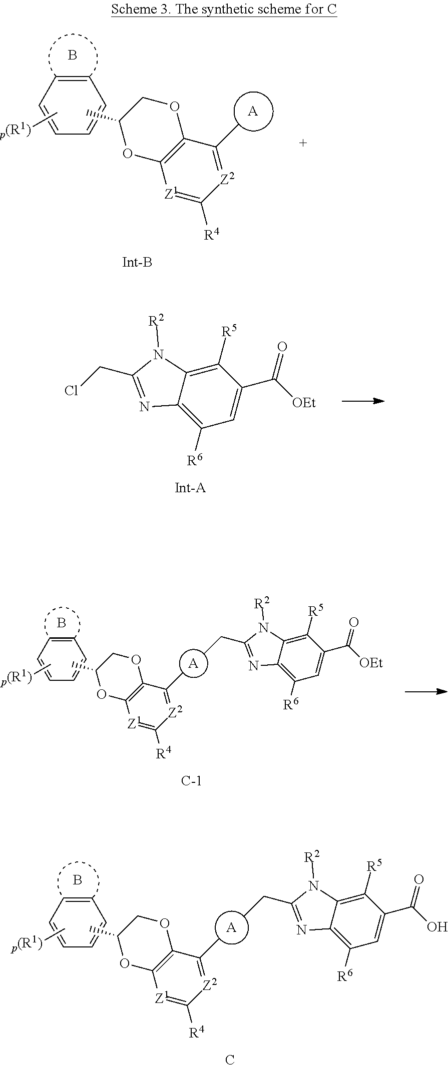

The reaction between Int-A and Int-B gives intermediate C-1. In certain embodiments, the reaction comprises DIEA. The hydrolysis of C-1 gives final product C. In certain embodiments, the reaction comprises LiOH.

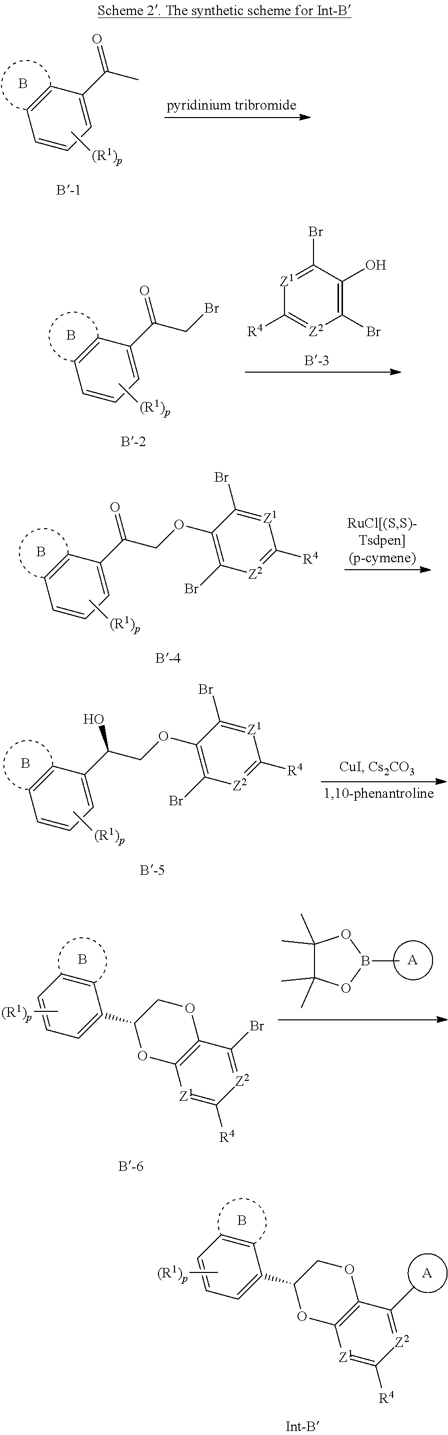

The synthesis of Int-B′ starts with bromination of acetophenone B′-1, for example using pyridinium tribromide, to give B′-2, which is then reacted with B′-3 to give B′-4. In certain embodiments, the reaction comprises a base, such as K₂CO₃. B′-4 is then enantioselectively reduced by RuCl[(S,S)-Tsdpen](p-cymene) and goes through a intramolecular coupling reaction to give B′-6. In certain embodiments, the reaction from B′-4 to B′-5 comprises TEA and formic acid. In certain embodiments, the reaction from B′-5 to B′-6 comprises toluene and heat. B′-6 is then coupled with benzylboronic acid pinacol ester B′-7 to give Int-B′. In certain embodiments, the reaction comprises Pd(dppf)Cl₂.

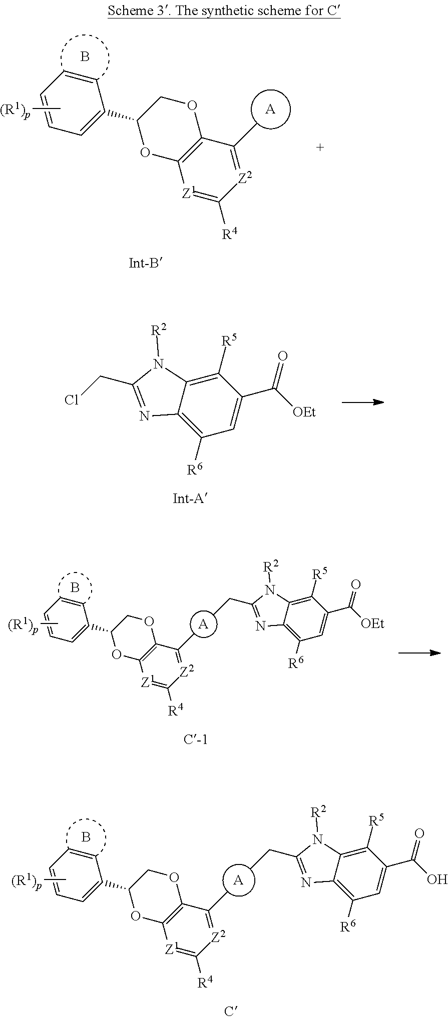

The reaction between Int-A′ and Int-B′ gives intermediate C′-1. In certain embodiments, the reaction comprises DIEA. The hydrolysis of C′-1 gives final product C′. In certain embodiments, the reaction comprises LiOH.

### Example A1

### 2-({4-[(2R)-2-(4-cyanophenyl)-2,3-dihydro-1,4-benzodioxin-5-yl]piperidin-1-yl}methyl)-1-[(oxolan-3-yl)methyl]-1H-1,3-benzodiazole-6-carboxylic acid (Compound 106)

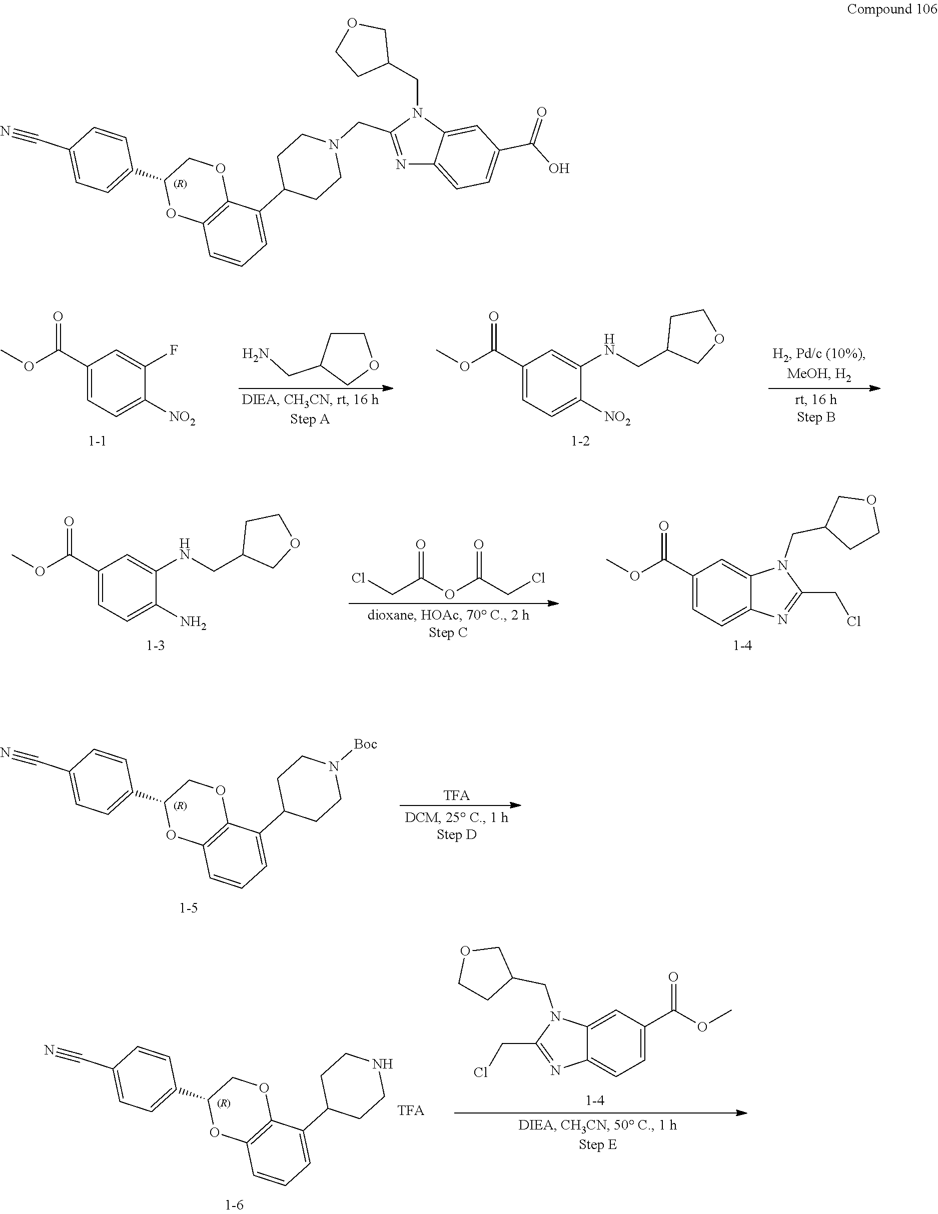

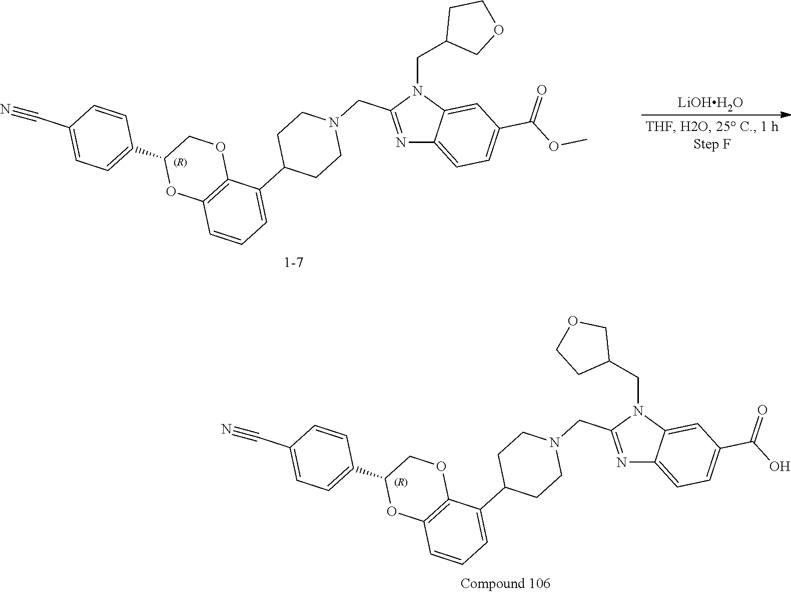

### Step A methyl 4-nitro-3-(((tetrahydrofuran-3-yl)methyl)amino)benzoate

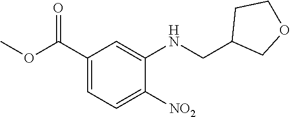 [SMILES: COC(=O)c1ccc([N+](=O)[O-])c(NCC2CCOC2)c1]

To a solution of methyl 3-fluoro-4-nitrobenzoate (1.514 g, 76 mmol), (tetrahydrofuran-3-yl)methanamine (1 g, 9.88 mmol) in CH₃CN (15 mL) was added DIEA (2.944 g, 22.8 mmol) and tetrahydro-3-furylmethanamine (2.306 g, 22.8 mmol). The reaction was stirred at room temperature for 1 hr. After the reaction was completed, the mixture was diluted with EtOAc (50 mL), washed with water (50 mL×4). The organic layer was dried over anhydrous Na₂SO₄, filtered and concentrated. The residue was purified by flash silica gel chromatography (40 g SepaFlash® Silica Flash Column, Eluent of 0~5% EtOAc/PE gradient@50 mL/min) to afford the methyl 4-nitro-3-(((tetrahydrofuran-3-yl)methyl)amino)benzoate (2.062 g, 96.8% yield). LC-MS: m/z 281.1 (M+H)⁺.

### Step B methyl 4-amino-3-(((tetrahydrofuran-3-yl)methyl)amino)benzoate

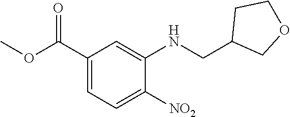 [SMILES: COC(=O)c1ccc([N+](=O)[O-])c(NCC2CCOC2)c1]

To a solution of methyl 4-nitro-3-(((tetrahydrofuran-3-yl)methyl)amino)benzoate (500 mg, 1.784 mmol) in MeOH (12 mL) and THF (10 mL) was added 10% Pd/C (100 mg). The resulting mixture was stirred at room temperature for 16 h under H₂ balloon. The mixture was filtered, the filtrate was concentrated to afford the methyl 4-amino-3-(((tetrahydrofuran-3-yl)methyl)amino)benzoate (490 mg, crude). LC-MS: m/z 251.2 (M+H)⁺.

### Step A methyl 2-(chloromethyl)-1-((tetrahydrofuran-3-yl)methyl)-1H-benzo[d]imidazole-6-carboxylate

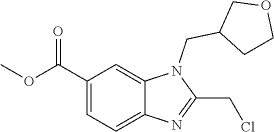 [SMILES: COC(=O)c1ccc2nc(CCl)n(CC3CCOC3)c2c1]

The mixture of methyl 4-amino-3-(((tetrahydrofuran-3-yl)methyl)amino)benzoate (590 mg, 2.357 mmol), 2-chloroacetic anhydride (403.01 mg, 2.357 mmol) in dioxane (2 mL) was stirred at 60° C. for 2 h. Then HOAc (0.2 mL) was added. The resulting reaction mixture was stirred at 70° C. for 2 hr After the reaction was completed, the mixture was diluted with DCM (50 mL), washed with sat. aq.

NaHCO₃. The organic phase was dried over anhydrous Na₂SO₄, filtered and concentrated. The residue was purified by flash silica gel chromatography (40 g SepaFlash® Silica Flash Column, Eluent of 0~25% EtOAc/PE gradient@50 mL/min) to afford the methyl 2-(chloromethyl)-1-((tetrahydrofuran-3-yl)methyl)-1H-benzo[d]imidazole-6-carboxylate (644 mg, 88.5% yield). LC-MS: m/z 309.1 (M+H)⁺.

### Step D (R)-4-(5-(piperidin-4-yl)-2,3-dihydrobenzo[b][1,4]dioxin-2-yl)benzonitrile TFA salt

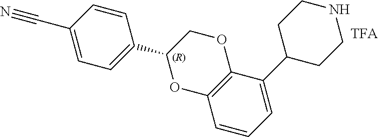

To the solution of tert-butyl (R)-4-(2-(4-cyanophenyl)-2,3-dihydrobenzo[b][1,4]dioxin-5-yl)piperidine-1-carboxylate (160 mg, 0.38 mmol) in DCM (3 mL) was added TFA (0.6 mL). The reaction mixture was stirred at 25° C. for 1 h. Then the solution was concentrated to give (R)-4-(5-(piperidin-4-yl)-2,3-dihydrobenzo[b][1,4]dioxin-2-yl)benzonitrile TFA salt (130 mg, crude). LC-MS: m/z 321.2 (M+H)⁺.

### Step E methyl 2-((4-((R)-2-(4-cyanophenyl)-2,3-dihydrobenzo[b][1,4]dioxin-5-yl)piperidin-1-yl)methyl)-1-((tetrahydrofuran-3-yl)methyl)-1H-benzo[d]imidazole-6-carboxylate

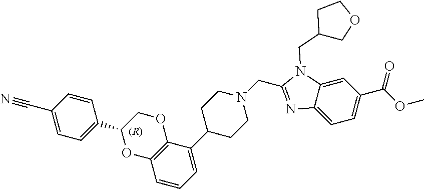

To a solution of (R)-4-(5-(piperidin-4-yl)-2,3-dihydrobenzo[b][1,4]dioxin-2-yl)benzonitrile TFA salt (182.89 mg, 0.421 mmol) in DMF (2 mL) was added DIEA (0.5 mL). The reaction mixture was stirred at room temperature for 2 min, then methyl 2-(chloromethyl)-1-((tetrahydrofuran-3-yl)methyl)-1H-benzo[d]imidazole-6-carboxylate (130 mg, 0.421 mmol) was added. The reaction was stirred at 50° C. for 1 h. After cooling, the mixture was diluted with EtOAc (50 mL), washed with water (50 mL×3). The organic phases were dried over anhydrous Na₂SO₄, filtered and concentrated. The residue was purified by flash silica gel chromatography (40 g SepaFlash® Silica Flash Column, Eluent of 0~25% EtOAc/PE gradient@50 mL/min) to afford the methyl 2-((4-((R)-2-(4-cyanophenyl)-2,3-dihydrobenzo[b][1,4]dioxin-5-yl)piperidin-1-yl)methyl)-1-((tetrahydrofuran-3-yl)methyl)-1H-benzo[d]imidazole-6-carboxylate (148 mg, 61.8% yield). LC-MS: m/z 593.4 (M+H)⁺.

### Step F 2-({4-[(2R)-2-(4-cyanophenyl)-2,3-dihydro-1,4-benzodioxin-5-yl]piperidin-1-yl}methyl)-1-[(oxolan-3-yl)methyl]-1H-1,3-benzodiazole-6-carboxylic acid (Compound 106)

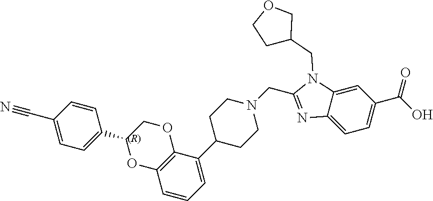

The mixture of methyl 2-((4-((R)-2-(4-cyanophenyl)-2,3-dihydrobenzo[b][1,4]dioxin-5-yl)piperidin-1-yl)methyl)-1-((tetrahydrofuran-3-yl)methyl)-1H-benzo[d]imidazole-6-carboxylate (100 mg, 0.169 mmol), LiOH·H₂O (20.25 mg, 0.844 mmol) in THF/H₂O (1 mL/0.2 mL) was stirred at 40° C. for 16 h. After cooling, the mixture was purified directly by prep. HPLC (Column: Sunfire C18, 19*250 mm*10 m; Mobile Phase A: Water (0.1% formic acid), B: CH₃CN; Flow rate: 20 mL/min; Gradient: 36% B to 38% B; Retention Time: 8.9-13.2 min of 16 min) to give Compound 106 (85.61 mg, 87.6% yield). LC-MS: m/z 579.4 (M+H)⁺. ¹H NMR (400 MHz, DMSO-d6) δ 12.99 (br s, 1H), 8.35 (s, 1H), 7.96-7.89 (m, 3H), 7.80 (d, J=8.4 Hz, 1H), 7.69 (d, J=8.0 Hz, 2H), 6.96-6.90 (m, 2H), 6.90-6.76 (m, 1H), 5.44-5.37 (m, 1H), 4.87-4.77 (m, 2H), 4.59-4.52 (m, 1H), 4.43 (d, J=8.0 Hz, 2H), 4.20-4.11 (m, 1H), 3.95-3.87 (m, 1H), 3.81-3.78 (m, 2H), 3.71-3.61 (m, 3H), 3.48-3.42 (m, 2H), 3.21-3.14 (m, 1H), 2.84-2.73 (m, 1H), 2.10-1.86 (m, 5H), 1.72-1.61 (m, 1H).

Example compounds 105 and 107 were synthesized using a similar procedure described in the Example A1 above using the appropriate materials.

### Example A2

### 2-({4-[(2R)-2-(4-cyanophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 104)

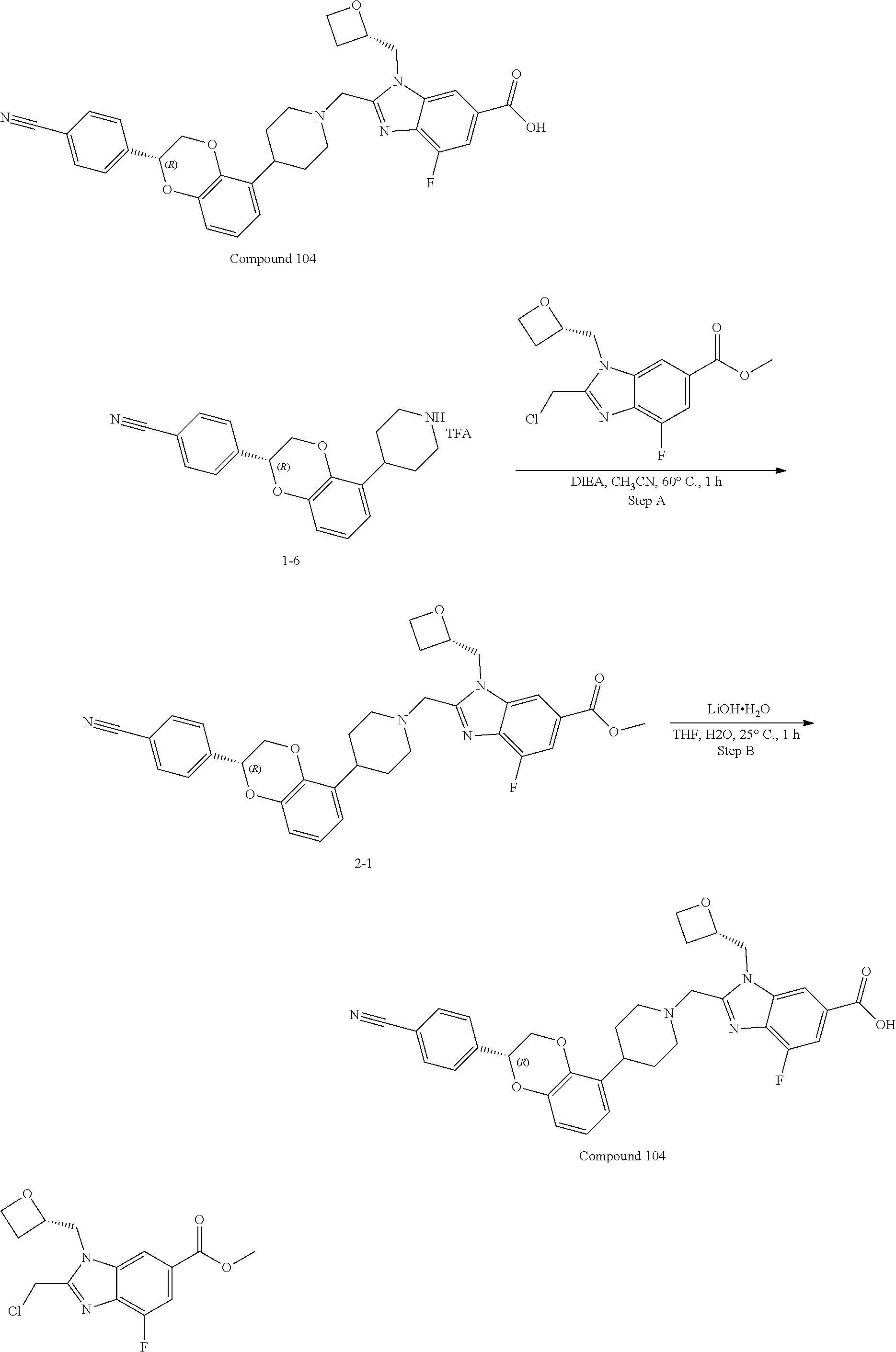

(S)-2-(chloromethyl)-4-fluoro-1-(oxetan-2-ylmethyl)-1H-benzo[d]imidazole-6-carboxylate was synthesized according to the procedures described for the preparation of intermediate 1-4 (step A B and C) by using (S)-oxetan-2-ylmethanamine and methyl 3,5-difluoro-4-nitrobenzoate in step A. LC-MS: m/z 313.2 (M+H)⁺.

### Step A methyl 2-((4-((R)-2-(4-cyanophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate

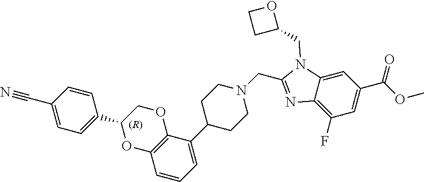

To the mixture of (R)-4-(5-(piperidin-4-yl)-2,3-dihydrobenzo[b][1,4]dioxin-2-yl)benzonitrile TFA salt (130 mg, crude) in CH₃CN (3.0 mL) was added DIEA (248 mg, 1.91 mmol) and methyl (S)-2-(chloromethyl)-4-fluoro-1-(oxetan-2-ylmethyl)-1H-benzo[d]imidazole-6-carboxylate (100 mg, 0.34 mmol). The reaction mixture was stirred at 60° C. for 1 h. After cooling and concentration, the residue was purified by flash silica gel chromatography (12 g SepaFlash® Silica Flash Column, Eluent of 0~5% MeOH/DCM gradient@50 mL/min) to give methyl 2-((4-((R)-2-(4-cyanophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate (205 mg, 90.5% yield over two steps). LC-MS: m/z 597.3 (M+H)⁺.

### Step B 2-({4-[(2R)-2-(4-cyanophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 104)

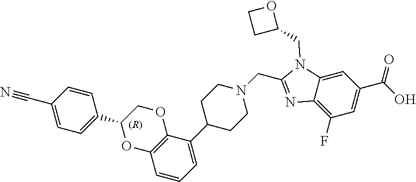

To a solution of methyl 2-((4-((R)-2-(4-cyanophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate (205 mg, 0.34 mmol) in THF/H₂O (V/V=4:1, 2.5 mL) was added LiOH·H₂O (58 mg, 1.38 mmol). The reaction mixture was stirred at 25° C. for 16 h. The reaction mixture was filtered and the filtrate was purified by prep. HPLC purification (Column: Sunfire C18, 19*250 mm*10 μm; Mobile Phase A: Water (0.1% formic acid), B: CH₃CN; Flow rate: 20 mL/min; Gradient: 32% B to 33% B; Retention Time: 8.4-11.1 min of 16 min) to give 2-({4-[(2R)-2-(4-cyanophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (80.2 mg, 40.1% yield). LC-MS: m/z 583.2 (M+H)⁺. ¹H NMR (400 MHz, DMSO-d6) δ 8.16 (s, 1H), 7.91 (d, J=8.4 Hz, 2H), 7.68 (d, J=8.4 Hz, 2H), 7.50 (d, J=11.2 Hz, 1H), 6.87-6.78 (m, 3H), 5.42-5.35 (m, 1H), 5.16-5.06 (m, 1H), 4.80 (dd, J₁=13.2 Hz, J₂=2.4 Hz, 1H), 4.73 (d, J=9.6 Hz, 1H), 4.58-4.46 (m, 2H), 4.42-4.33 (m, 1H), 4.13-4.05 (m, 1H), 3.95 (d, J=13.6 Hz, 1H), 3.82 (d, J=13.6 Hz, 1H), 3.05-2.96 (m, 1H), 2.91-2.81 (m, 2H), 2.77-2.65 (m, 1H), 2.48-2.38 (m, 1H), 2.32-2.13 (m, 2H), 1.81-1.71 (m, 1H), 1.69-1.55 (m, 3H). ¹⁹F NMR (377 MHz, DMSO-d6) 3-129.16.

### Example A3

### 2-({4-[(2R)-2-(4-cyanophenyl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (Compound 118)

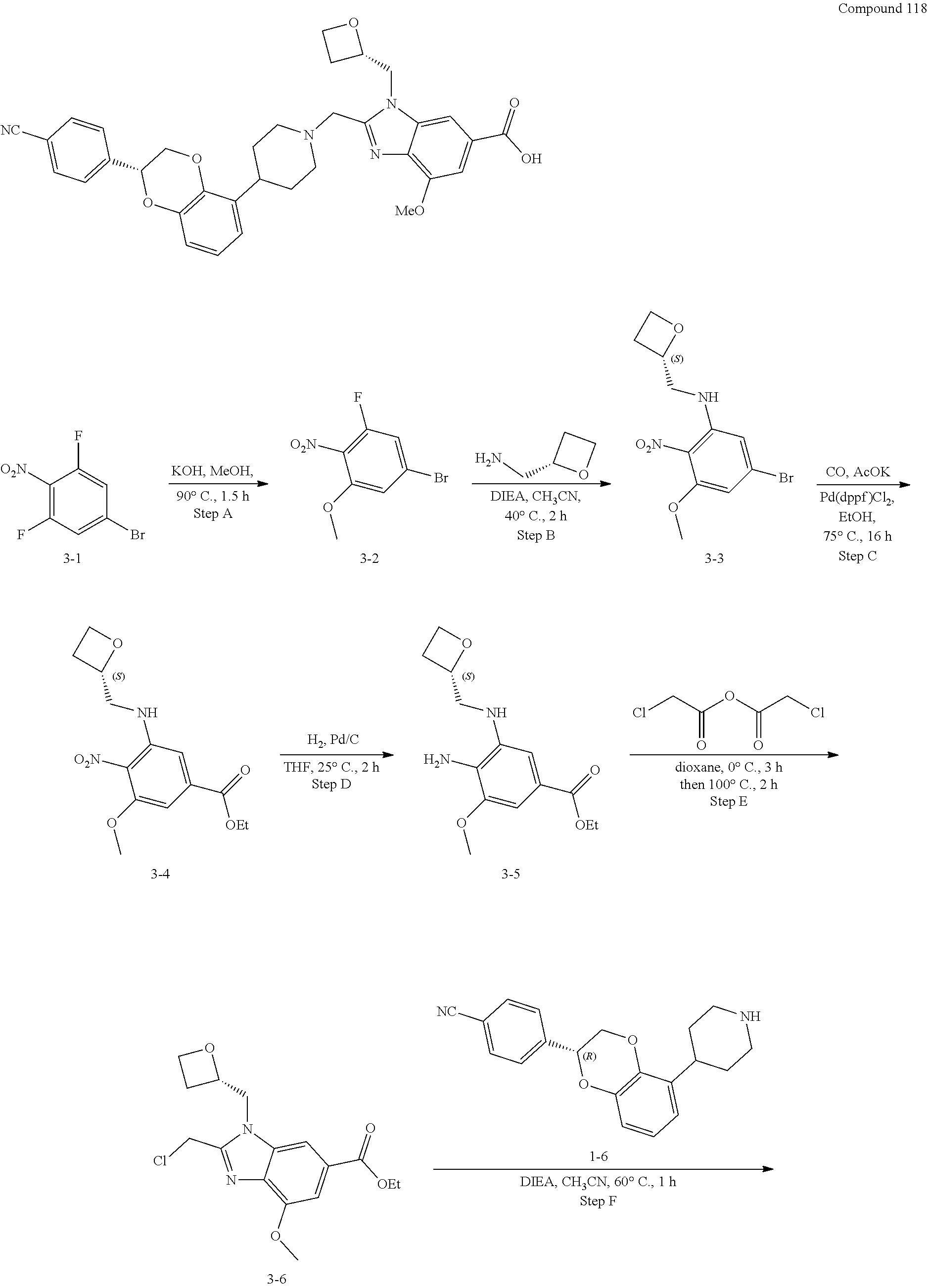

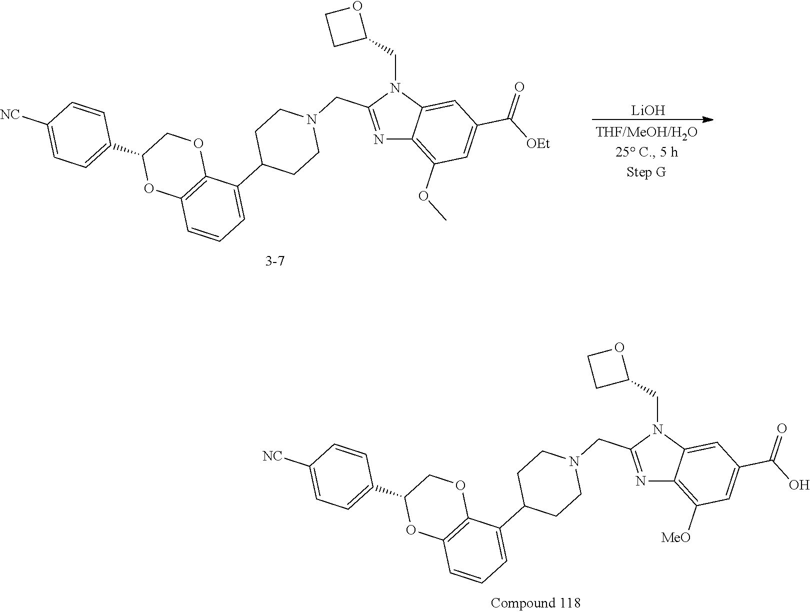 [SMILES: CCOC(=O)c1cc(OC)c2nc(CN3CCC(c4cccc5c4OC[C@@H](c4ccc(C#N)cc4)O5)CC3)n(C[C@@H]3CCO3)c2c1 ; "LiOH" ; COc1cc(C(=O)O)cc2c1nc(CN1CCC(c3cccc4c3OC[C@@H](c3ccc(C#N)cc3)O4)CC1)n2C[C@@H]1CCO1]

### Step A 5-bromo-1-fluoro-3-methoxy-2-nitrobenzene

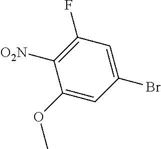

The mixture of 5-bromo-1,3-difluoro-2-nitrobenzene (10.0 g, 42.02 mmol) and KOH (2.47 g, 44.12 mmol) in MeOH (100 mL) was stirred at 90° C. for 1.5 h. After cooling, the reaction mixture was diluted with EtOAc (400 mL), washed with water (300 mL×2) and brine (100 mL). The organic layer was dried over anhydrous Na₂SO₄, filtered and concentrated. The residue was purified by flash silica gel chromatography (120 g SepaFlash® Silica Flash Column, Eluent of 0~10% EtOAc/PE gradient@100 mL/min) to give 5-bromo-1-fluoro-3-methoxy-2-nitrobenzene (5.6 g, 53.3% yield).

### Step B (S)-5-bromo-3-methoxy-2-nitro-N-(oxetan-2-ylmethyl)aniline

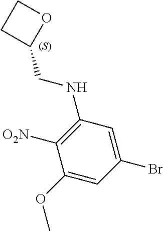

To a solution of 5-bromo-1-fluoro-3-methoxy-2-nitrobenzene (10.8 g, 43.37 mmol) in CH₃CN (130 mL) was added DIEA (16.81 g, 130.12 mmol) and (S)-oxetan-2-ylmethanamine (3.77 g, 43.37 mmol). The resulting mixture was stirred at 40° C. for 2 h. After cooling, the reaction mixture was diluted with EtOAc (300 mL), washed with water (300 mL×2) and brine (100 mL). The organic layer was dried over anhydrous Na₂SO₄, filtered and concentrated. The residue was purified by flash silica gel chromatography (120 g SepaFlash® Silica Flash Column, Eluent of 0~25% EtOAc/PE gradient@100 mL/min) to give (S)-5-bromo-3-methoxy-2-nitro-N-(oxetan-2-ylmethyl)aniline (10.4 g, 75.9% yield).

LC-MS: m/z 317.2 (M+H)⁺.

### Step C ethyl (S)-3-methoxy-4-nitro-5-((oxetan-2-ylmethyl)amino)benzoate

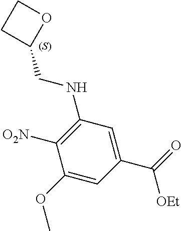

To the mixture of (S)-5-bromo-3-methoxy-2-nitro-N-(oxetan-2-ylmethyl)aniline (10.4 g, 32.91 mmol) in EtOH (120 mL), was added AcOK (6.46 g, 65.8 mmol) and Pd(dppf)Cl₂ (2.41 g, 3.29 mmol). The reaction mixture was stirred at 75° C. for 16 h under CO atmosphere. After the reaction was completed, the mixture was concentrated. The residue was purified by flash silica gel chromatography (120 g SepaFlash® Silica Flash Column, Eluent of 10~25% EtOAc/PE gradient@100 mL/min) to give ethyl (S)-3-methoxy-4-nitro-5-((oxetan-2-ylmethyl)amino)benzoate (6.1 g, 59.8% yield). LC-MS: m/z 310.9 (M+H)⁺.

### Step D ethyl (S)-4-amino-3-methoxy-5-((oxetan-2-ylmethyl)amino)benzoate

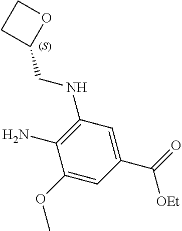

To a solution of ethyl (S)-3-methoxy-4-nitro-5-((oxetan-2-ylmethyl)amino)benzoate (6.1 g, 19.68 mmol) in THF (60 mL) was added 10% Pd/C (915 mg). The reaction mixture was degassed and charged with H₂ three times and then stirred at 25° C. for 2 h under H₂ balloon. The reaction mixture was filtered, the filtrate was concentrated. The residue was purified by flash silica gel chromatography (120 g SepaFlash® Silica Flash Column, Eluent of 0~5% MeOH/DCM gradient@100 mL/min) to give ethyl (S)-4-amino-3-methoxy-5-((oxetan-2-ylmethyl)amino)benzoate (5.05 g, 91.7% yield). LC-MS: m/z 281.0 (M+H)⁺.

### Step E ethyl (S)-4-(2-chloroacetamido)-3-methoxy-5-((oxetan-2-ylmethyl)amino)benzoate

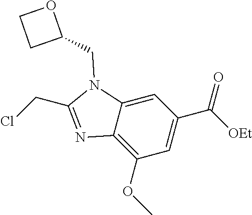 [SMILES: CCOC(=O)c1cc(OC)c2nc(CCl)n(C[C@@H]3CCO3)c2c1]

To a solution of ethyl (S)-4-amino-3-methoxy-5-((oxetan-2-ylmethyl)amino)benzoate (166 mg, 0.59 mmol) in dioxane (3 mL) was added 2-chloroacetic anhydride (101 mg, 0.59 mmol) at 0° C. The reaction mixture was stirred at 25° C. for 3 h. Then AcOH (0.5 ml) was added, the reaction mixture was stirred to 100° C. and continued to stir for another 2 h. After cooling, the mixture was diluted with EtOAc (40 mL), washed with water (50 mL×2) and brine (40 mL). The organic layer was dried over anhydrous Na₂SO₄, filtered and concentrated. The residue was purified by flash silica gel chromatography (12 g SepaFlash® Silica Flash Column, Eluent of 0~5% MeOH/DCM gradient@50 mL/min) to give ethyl (S)-2-(chloromethyl)-4-methoxy-1-(oxetan-2-ylmethyl)-1H-benzo[d]imidazole-6-carboxylate (120 mg, 60% yield). LC-MS: m/z 338.8 (M+H)⁺.

### Step F ethyl 2-((4-((R)-2-(4-cyanophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate

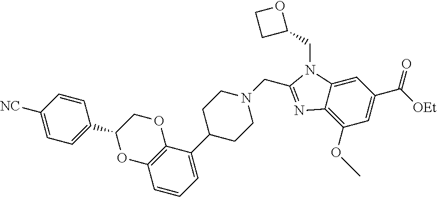 [SMILES: CCOC(=O)c1cc(OC)c2nc(CN3CCC(c4cccc5c4OC[C@@H](c4ccc(C#N)cc4)O5)CC3)n(C[C@@H]3CCO3)c2c1]

To a solution of ethyl (S)-2-(chloromethyl)-4-methoxy-1-(oxetan-2-ylmethyl)-1H-benzo[d]imidazole-6-carboxylate (120 mg, 0.36 mmol) in CH₃CN was added DIEA (465 mg, 3.6 mmol) and (R)-4-(5-(piperidin-4-yl)-2,3-dihydrobenzo[b][1,4]dioxin-2-yl)benzonitrile TFA salt (156 mg, 0.36 mmol). The reaction mixture was stirred at 60° C. for 1 h. The reaction mixture was diluted with EtOAc (40 mL) and washed with brine (30 mL×3). The organic layer was dried over anhydrous Na₂SO₄, filtered and concentrated. The residue was purified by flash silica gel chromatography (12 g SepaFlash® Silica Flash Column, Eluent of 0~5% MeOH/DCM gradient@50 mL/min) to give ethyl 2-((4-((R)-2-(4-cyanophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate (133 mg, 60.4% yield). LC-MS: m/z 622.8 (M+H)⁺.

### Step G 2-({4-[(2R)-2-(4-cyanophenyl)-2,3-dihydro-1,4-benzodioxin-5-yl]piperidin-1-yl}methyl)-4-methoxy-1-{[(2S)-oxetan-2-yl]methyl-}-1H-1,3-benzodiazole-6-carboxylic acid (Compound 118)

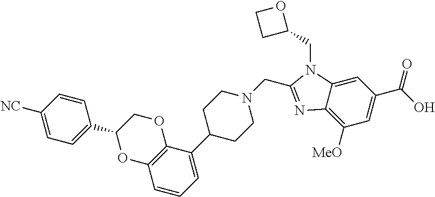 [SMILES: COc1cc(C(=O)O)cc2c1nc(CN1CCC(c3cccc4c3OC[C@@H](c3ccc(C#N)cc3)O4)CC1)n2C[C@@H]1CCO1]

To a solution of ethyl 2-((4-((R)-2-(4-cyanophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate (133 mg, 0.21 mmol) in THE/MeOH/H₂O (V/V/V=4:1:1, 1 mL) was added LiOH·H₂O (36 mg, 0.85 mmol). The resulting mixture was stirred at 25° C. for 1 h. The solution was filtered and the filtrate was purified by prep. HPLC purification (Column: Pursuit XRs C18, 19*250 mm*10 μm; Mobile Phase A: Water (0.1% formic acid), B: CH₃CN; Flow rate: 20 mL/min; Gradient: 35% B to 35% B; Retention Time: 6.38-8.5 min) to give 2-({4-[(2R)-2-(4-cyanophenyl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (24.0 mg, 18.9% yield). LC-MS: m/z 595.1 (M+H)⁺. ¹H NMR (400 MHz, DMSO-d6) δ 12.80 (br s, 1H), 7.95-7.86 (m, 3H), 7.68 (d, J=8.4 Hz, 2H), 7.26 (s, 1H), 6.88-6.79 (m, 3H), 5.41-5.35 (m, 1H), 5.13-5.04 (m, 1H), 4.80-4.72 (m, 1H), 4.68-4.60 (m, 1H), 4.58-4.46 (m, 2H), 4.39-4.30 (m, 1H), 4.14-4.05 (m, 1H), 3.95 (s, 3H), 3.90 (d, J=13.2 Hz, 1H), 3.78 (d, J=13.6 Hz, 1H), 3.03-2.92 (m, 1H), 2.80-2.70 (m, 2H), 2.72-2.64 (m, 1H), 2.45-2.32 (m, 1H), 2.26-2.10 (m, 2H), 1.80-1.53 (m, 4H).

### Example A4

### 2-({4-[(2R)-2-(4-chlorophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 111)

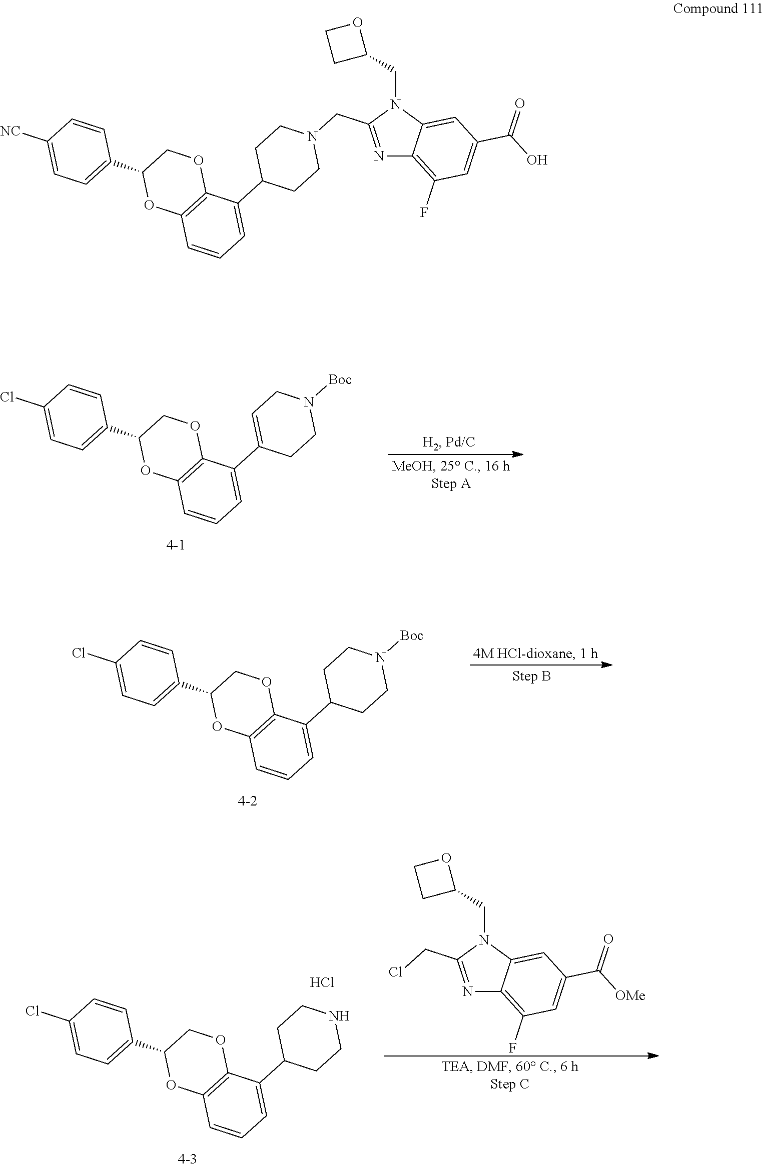 [SMILES: N#Cc1ccc([C@@H]2COc3c(cccc3C3CCN(Cc4nc5c(F)cc(C(=O)O)cc5n4C[C@@H]4CCO4)CC3)O2)cc1 ; CC(C)(C)OC(=O)N1CC=C(c2cccc3c2OC[C@@H](c2ccc(Cl)cc2)O3)CC1 ; CC(C)(C)OC(=O)N1CCC(c2cccc3c2OC[C@@H](c2ccc(Cl)cc2)O3)CC1 ; Clc1ccc([C@@H]2COc3c(cccc3C3CCNCC3)O2)cc1 ; Cl ; COC(=O)c1cc(F)c2nc(CCl)n(C[C@@H]3CCO3)c2c1]

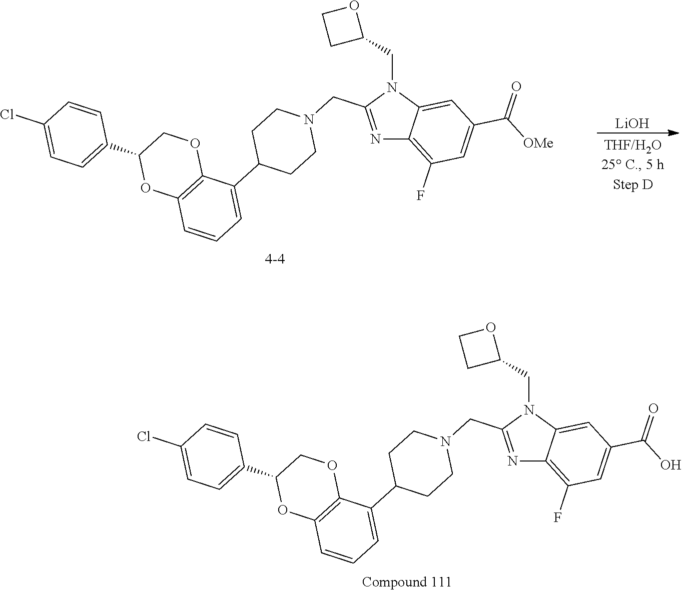 [SMILES: COC(=O)c1cc(F)c2nc(CN3CCC(c4cccc5c4OC[C@@H](c4ccc(Cl)cc4)O5)CC3)n(C[C@@H]3CCO3)c2c1 ; "LiOH" ; O=C(O)c1cc(F)c2nc(CN3CCC(c4cccc5c4OC[C@@H](c4ccc(Cl)cc4)O5)CC3)n(C[C@@H]3CCO3)c2c1]

### Step A tert-butyl (R)-4-(2-(4-chlorophenyl)-2,3-dihydrobenzo[b][1,4]dioxin-5-yl)piperidine-1-carboxylate

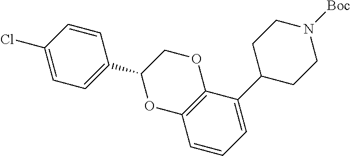 [SMILES: CC(C)(C)OC(=O)N1CCC(c2cccc3c2OC[C@@H](c2ccc(Cl)cc2)O3)CC1]

To the mixture of tert-butyl (R)-4-(2-(4-chlorophenyl)-2,3-dihydrobenzo[b][1,4]dioxin-5-yl)-3,6-dihydropyridine-1(2H)-carboxylate (50 mg, 0.117 mmol) in MeOH (1 mL), was added 5% Pd/C (10 mg). The reaction mixture was degassed and charged with H₂ three times and then stirred at 25° C. for 16 h. After the reaction was completed, the mixture was filtered and concentrated to give tert-butyl (R)-4-(2-(4-chlorophenyl)-2,3-dihydrobenzo[b][1,4]dioxin-5-yl)piperidine-1-carboxylate (50 mg, 99.5% yield). LC-MS: m/z 374.2 (M+H-t-Bu)⁺.

### Step B (R)-4-(2-(4-chlorophenyl)-2,3-dihydrobenzo[b][1,4]dioxin-5-yl)piperidine HCl salt

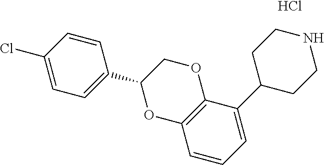 [SMILES: Cl ; Clc1ccc([C@@H]2COc3c(cccc3C3CCNCC3)O2)cc1]

The mixture of tert-butyl (R)-4-(2-(4-chlorophenyl)-2,3-dihydrobenzo[b][1,4]dioxin-5-yl)piperidine-1-carboxylate (50 mg, 0.116 mmol) in 4 M HCl (gas)/dioxane (2 mL) was stirred at 25° C. for 1 h. After the reaction was completed, the mixture was concentrated to give (R)-4-(2-(4-chlorophenyl)-2,3-dihydrobenzo[b][1,4]dioxin-5-yl)piperidine HCl salt (50 mg crude). LC-MS: m/z 330.2 (M+H)⁺.

### Step C methyl 2-((4-((R)-2-(4-chlorophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate

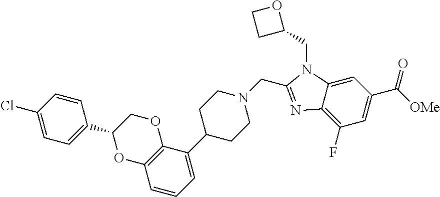 [SMILES: COC(=O)c1cc(F)c2nc(CN3CCC(c4cccc5c4OC[C@@H](c4ccc(Cl)cc4)O5)CC3)n(C[C@@H]3CCO3)c2c1]

To a solution of (R)-4-(2-(4-chlorophenyl)-2,3-dihydrobenzo[b][1,4]dioxin-5-yl)piperidine HCl salt (50 mg, crude) in DMF (1 mL) was added TEA (92 mg, 0.91 mmol). After being stirred for 2 minutes, methyl (S)-2-(chloromethyl)-4-fluoro-1-(oxetan-2-ylmethyl)-1H-benzo[d]imidazole-6-carboxylate (28 mg, 0.091 mmol) was added. The reaction mixture was stirred at 60° C. for 6 h. The reaction mixture was concentrated. The residue was purified by prep. TLC purification (PE/EtOAc=2/1) to give methyl 2-((4-((R)-2-(4-chlorophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate (44 mg, 62.9% yield). LC-MS: m/z 606.3 (M+H)⁺.

### Step D 2-({4-[(2R)-2-(4-chlorophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 111)

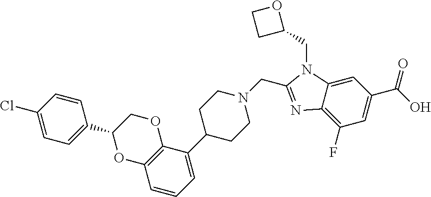 [SMILES: O=C(O)c1cc(F)c2nc(CN3CCC(c4cccc5c4OC[C@@H](c4ccc(Cl)cc4)O5)CC3)n(C[C@@H]3CCO3)c2c1]

To the mixture of methyl 2-((4-((R)-2-(4-chlorophenyl)-2,3-dihydrobenzo[b][1,4]dioxin-5-yl)piperidin-1-yl)methyl)-4-fluoro-1-(((S)-oxetan-2-yl)methyl)-1H-benzo[d]imidazole-6-carboxylate (44 mg, 0.073 mmol) in THF (1 mL) and H₂O (0.2 mL), was added LiOH (15 mg, 0.363 mmol). The reaction mixture was stirred at 25° C. for 16 h and then concentrated, the residue was purified by prep. HPLC purification (Column: Sunfire C18, 19*250 mm*10 m; Mobile Phase A: Water (0.1% formic acid), B: CH₃CN; Flow rate: 20 mL/min; Gradient: 33% B to 43% B; Retention Time: 9.0-11.1 min) to give 2-({4-[(2R)-2-(4-chlorophenyl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (17.88 mg, 41.6% yield). LC-MS: m/z 592.2 (M+H)⁺. H NMR (400 MHz, DMSO-d6) δ 12.96 (br s, 1H), 8.16 (s, 1H), 7.58-7.45 (m, 5H), 6.89-6.78 (m, 3H), 5.25 (dd, J₁=8.4 Hz, J₂=2.4 Hz, 1H), 5.16-5.06 (m, 1H), 4.89-4.78 (m, 1H), 4.74-4.65 (m, 1H), 4.56-4.45 (m, 2H), 4.42-4.33 (m, 1H), 4.12-4.01 (m, 1H), 3.95 (d, J=14.0 Hz, 1H), 3.82 (d, J=13.6 Hz, 1H), 3.06-2.99 (m, 1H), 2.96-2.80 (m, 2H), 2.78-2.64 (m, 1H), 2.47-2.37 (m, 1H), 2.31-2.12 (m, 2H), 1.81-1.53 (m, 4H). ¹⁹F NMR (377 MHz, DMSO-d6) δ−129.14.

### Example A5

### 2-({4-[(2R)-2-(4-chlorophenyl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (Compound 119)

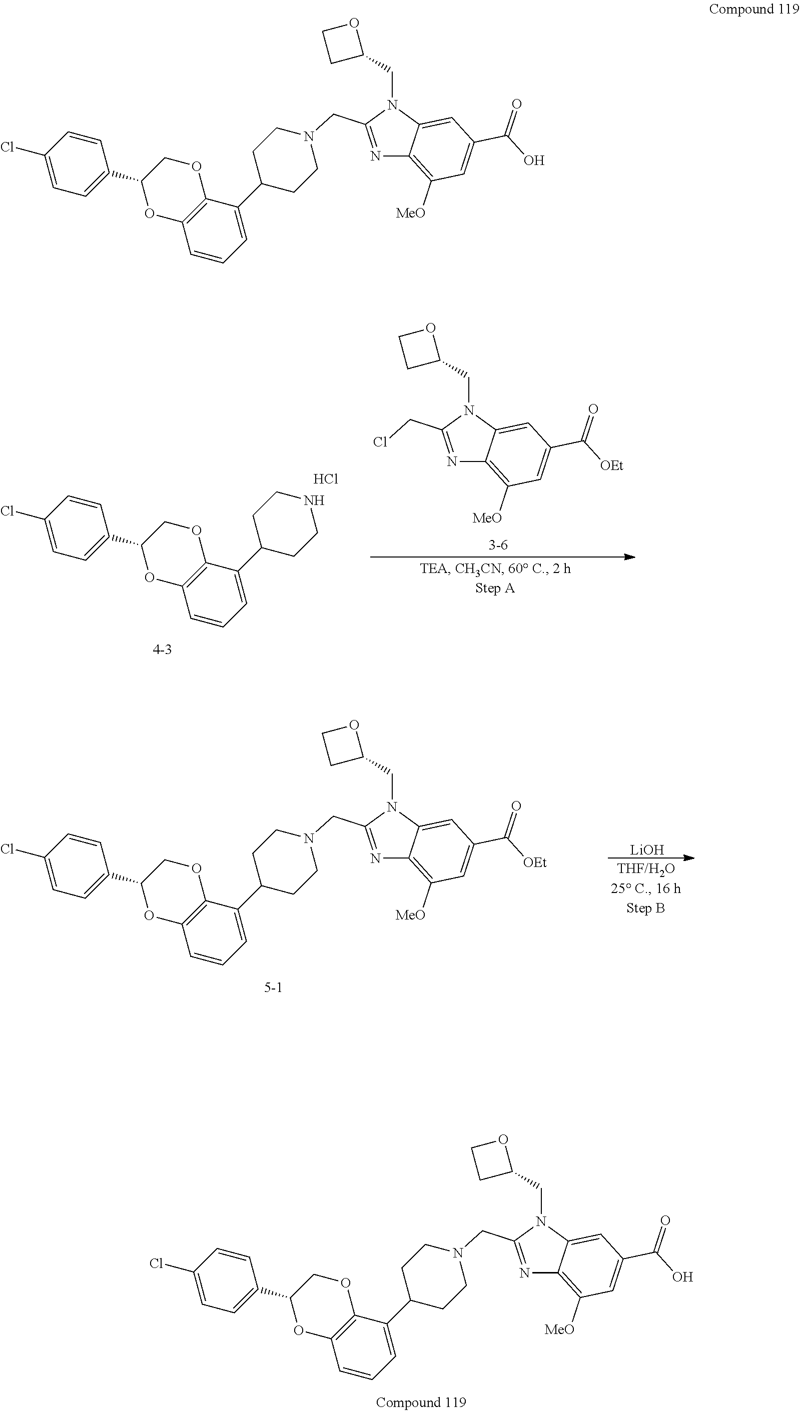

### Step A ethyl 2-((4-((R)-2-(4-chlorophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate

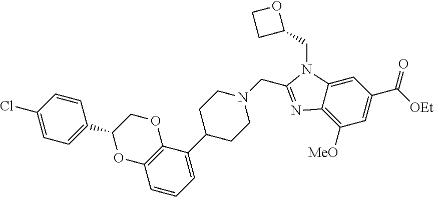 [SMILES: CCOC(=O)c1cc(OC)c2nc(CN3CCC(c4cccc5c4OC[C@@H](c4ccc(Cl)cc4)O5)CC3)n(C[C@@H]3CCO3)c2c1]

To the mixture of (R)-4-(2-(4-chlorophenyl)-2,3-dihydrobenzo[b][1,4]dioxin-5-yl)piperidine HCl salt (55 mg, 0.15 mmol) and ethyl (S)-2-(chloromethyl)-4-methoxy-1-(oxetan-2-ylmethyl)-1H-benzo[d]imidazole-6-carboxylate (60 mg, 0.18 mmol) in CH₃CN (6 mL), was added TEA (172 mg, 0.75 mmol). The reaction mixture was stirred at 60° C. for 2 h. After the reaction was completed, the mixture was concentrated. The residue was purified by flash silica gel chromatography (12 g SepaFlash® Silica Flash Column, Eluent of 0~50% EtOAc/PE gradient@50 mL/min) to give ethyl 2-((4-((R)-2-(4-chlorophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate (77 mg, 95.9% yield). LC-MS: m/z 632.1 (M+H)⁺.

### Step B 2-({4-[(2R)-2-(4-chlorophenyl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (Compound 119)

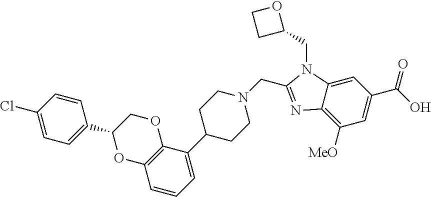 [SMILES: COc1cc(C(=O)O)cc2c1nc(CN1CCC(c3cccc4c3OC[C@@H](c3ccc(Cl)cc3)O4)CC1)n2C[C@@H]1CCO1]

To the mixture of ethyl 2-((4-((R)-2-(4-chlorophenyl)-2,3-dihydrobenzo[b][1,4]dioxin-5-yl)piperidin-1-yl)methyl)-4-methoxy-1-(((S)-oxetan-2-yl)methyl)-1H-benzo[d]imidazole-6-carboxylate (77 mg, 0.12 mmol) in H₂O (4 mL) and THF (12 mL), was added LiOH·H₂O (26 mg, 0.61 mmol). The reaction mixture was stirred at 25° C. for 16 h. After the reaction was completed, the reaction mixture was diluted with H₂O (10 mL), acidified with 1 M aq. HCl to pH=5-6, extracted with EtOAc (10 mL×2). The combined organic layers were washed with brine, dried over anhydrous Na₂SO₄, filtered and concentrated. The residue was purified by prep. HPLC purification (Column: Atlantis TM T3 Prep OBD TM C18, 19*250 mm*10 μm; Mobile Phase A: Water (0.1% formic acid), B: CH₃CN; Flow rate: 20 mL/min; Gradient: 39% B to 49% B; Retention Time: 6.5-8.2 min) to give 2-({4-[(2R)-2-(4-chlorophenyl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (19.81 mg, 27.5% yield). LC-MS: m/z 604.1 (M+H)⁺.

¹H NMR (400 MHz, DMSO-d6) δ 12.74 (s, 1H), 7.90 (s, 1H), 7.50 (s, 4H), 7.26 (s, 1H), 6.82-6.81 (m, 3H), 5.26-5.24 (m, 1H), 5.11-5.07 (m, 1H), 4.79-4.73 (m, 1H), 4.65-4.62 (m, 1H), 4.51-4.48 (m, 2H), 4.35-4.33 (m, 1H), 4.08-4.05 (m, 1H), 3.95 (s, 3H), 3.80-3.75 (m, 1H), 3.03-2.96 (m, 1H), 2.89-2.83 (m, 2H), 2.73-2.67 (m, 2H), 2.25-2.05 (s, 3H), 1.79-1.51 (m, 4H).

### Example A6

### 2-({4-[(2R)-2-(4-chloro-1-benzofuran-7-yl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (Compound 120)

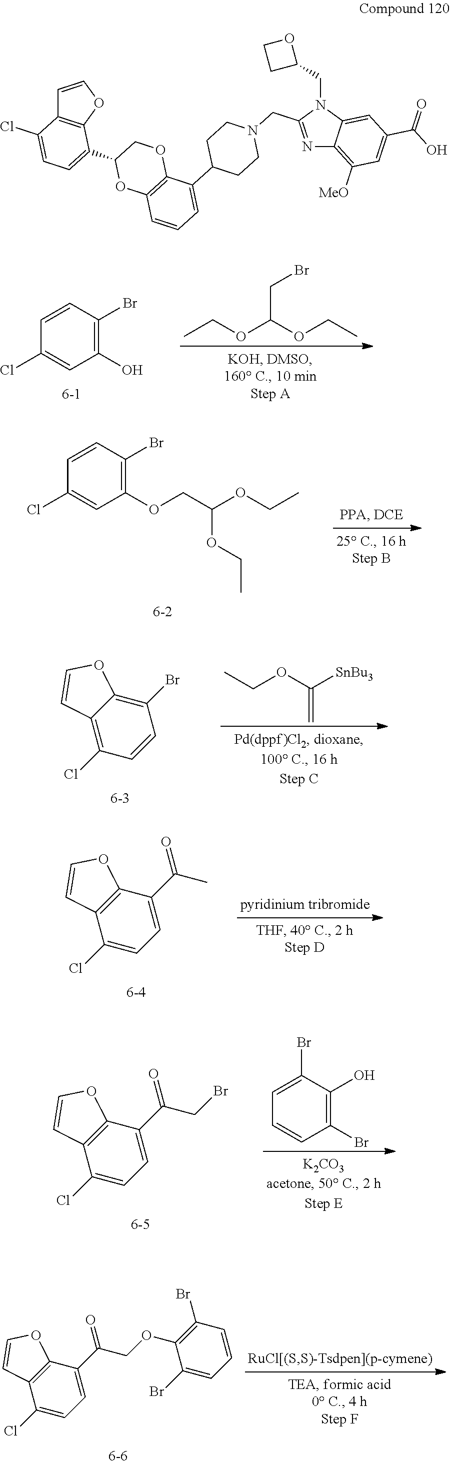

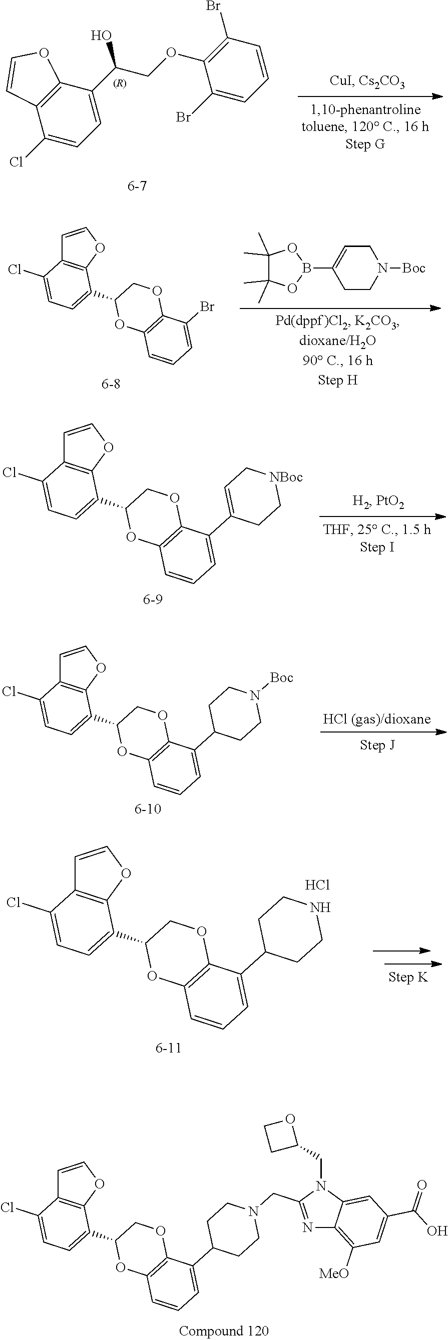

### Step A 1-bromo-4-chloro-2-(2,2-diethoxyethoxy)benzene

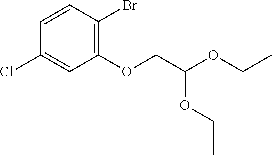 [SMILES: CCOC(COc1cc(Cl)ccc1Br)OCC]

To the mixture of 2-bromo-5-chlorophenol (5.00 g, 24.3 mmol), 1-bromo-4-chloro-2-(2,2-diethoxyethoxy)benzene (7.14 g, 36.24 nmol) in DMSO (30 mL) was added KOH (2.03 g, 36.25 nmol). The reaction mixture was stirred at 160° C. for 10 min. After cooling to 25° C., the mixture was poured into water (300 mL), extracted with ethyl acetate (300 mL×5). The organic layer was combined and washed with brine, dried over anhydrous Na₂SO₄, filtered and concentrated. The residue was purified by flash silica gel chromatography (120 g SepaFlash® Silica Flash Column, Eluent of 0~10% EtOAc/PE gradient@100 mL/min) to give 1-bromo-4-chloro-2-(2,2-diethoxyethoxy)benzene (7.1 g, 91.3% yield).

### Step B 7-bromo-4-chlorobenzofuran

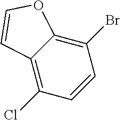 [SMILES: Clc1ccc(Br)c2occc12]

To a mixture of 1-bromo-4-chloro-2-(2,2-diethoxyethoxy)benzene (7.1 g, 22.05 mmol) in DCE (50 ml) was added PPA (10.8 g, 31.95 mmol). The reaction mixture was stirred at 25° C. for 16 h. The reaction mixture was quenched with sat. aq. NaHCO₃ (200 mL), extracted with ethyl acetate (200 mL×3). The organic layer was combined and washed with brine, dried over anhydrous Na₂SO₄, filtered and concentrated. The residue was purified by flash silica gel chromatography (120 g SepaFlash® Silica Flash Column, Eluent of 0~10% EtOAc/PE gradient@100 mL/min) to give 7-bromo-4-chlorobenzofuran (2.1 g, 41.4% yield).

### Step C 1-(4-chlorobenzofuran-7-yl)ethan-1-one

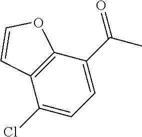

To the mixture of 7-bromo-4-chlorobenzofuran (1.9 g, 8.26 mmol) and tributyl(1-ethoxyvinyl)stannane (3.6 g, 9.97 mmol) in dioxane (20 mL) was added Pd(dppf)Cl₂ (604 mg, 0.83 mmol). The reaction mixture was stirred at 100° C. for 16 h. After cooling, the mixture was quenched with 1 M aq. HCl (20 mL) and stirred at 25° C. for 1 h. Then the reaction mixture was filtered and concentrated. The residue was purified by flash silica gel chromatography (40 g SepaFlash® Silica Flash Column, Eluent of 0~5% EtOAc/PE gradient@50 mL/min) to give 1-(4-chlorobenzofuran-7-yl)ethan-1-one (1.37 g, 85.6% yield). LC-MS: m/z 195.1 (M+H)⁺.

### Step D 2-bromo-1-(4-chlorobenzofuran-7-yl)ethan-1-one

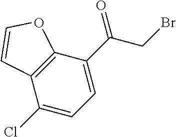 [SMILES: O=C(CBr)c1ccc(Cl)c2ccoc12]

To a mixture of 1-(4-chlorobenzofuran-7-yl)ethan-1-one (1.17 g, 6.03 mmol) in THF (12.0 mL) was added pyridinium tribromide (1.83 g, 5.72 mmol). The reaction mixture was stirred at 40° C. for 2 h. After the reaction was completed, the mixture was quenched with sat. aq. NaHCO₃ (40 mL), extracted with ethyl acetate (40 mL×3). The organic layer was combined and washed with brine, dried over anhydrous Na₂SO₄, filtered and concentrated. The residue was purified by flash silica gel chromatography (40 g SepaFlash® Silica Flash Column, Eluent of 10-20% EtOAc/PE gradient@50 mL/min) to give 2-bromo-1-(4-chlorobenzofuran-7-yl)ethan-1-one (790 mg, 48.1% yield). LC-MS: m/z 273.0 (M+H)⁺.

### Step E 1-(4-chlorobenzofuran-7-yl)-2-(2,6-dibromophenoxy)ethan-1-one

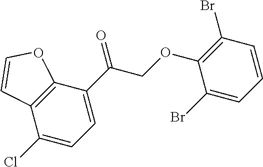 [SMILES: O=C(COc1c(Br)cccc1Br)c1ccc(Cl)c2ccoc12]

The mixture of 2-bromo-1-(4-chlorobenzofuran-7-yl)ethan-1-one (790 mg, 2.90 mmol), 2,6-dibromophenol (1.1 g, 4.37 mmol) and K₂CO₃ (1.2 g, 8.69 mmol) in acetone (10 mL) was stirred at 50° C. for 2 h. After the reaction was completed, the mixture was concentrated. The residue was purified by flash silica gel chromatography (24 g SepaFlash® Silica Flash Column, Eluent of 10~20% EtOAc/PE gradient@50 mL/min) to give 1-(4-chlorobenzofuran-7-yl)-2-(2,6-dibromophenoxy)ethan-1-one (580 mg, 45.3% yield). LC-MS: m/z 442.7 (M+H)⁺.

### Step F (R)-1-(4-chlorobenzofuran-7-yl)-2-(2,6-dibromophenoxy)ethan-1-ol

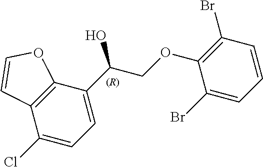

The mixture of RuCl[(S,S)-Tsdpen](p-cymene) (53 mg, 0.083 mmol) in TEA (18.5 mL) and formic acid (3.7 mL) was stirred at 0° C. for 1 h. Then 1-(4-chlorobenzofuran-7-yl)-2-(2,6-dibromophenoxy)ethan-1-one (370 mg, 0.84 mmol) was added. The resulting reaction mixture was stirred at 0° C. for 4 h. The solution was quenched with H₂O (40 mL) and extracted with ethyl acetate (30 mL×3). The organic layer was combined and washed with brine, dried over anhydrous Na₂SO₄, filtered and concentrated. The residue was purified by flash silica gel chromatography (12 g SepaFlash® Silica Flash Column, Eluent of 0~25% EtOAc/PE gradient@50 mL/min) to give (R)-1-(4-chlorobenzofuran-7-yl)-2-(2,6-dibromophenoxy)ethan-1-ol (270 mg, 72.6% yield). LC-MS: m/z 447.2 (M+H)⁺.

### Step G (R)-5-bromo-2-(4-chlorobenzofuran-7-yl)-2,3-dihydrobenzo[b][1,4]dioxine

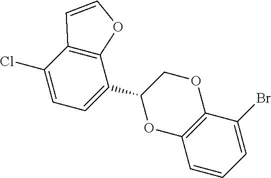 [SMILES: Clc1ccc([C@@H]2COc3c(Br)cccc3O2)c2occc12]

The mixture of (R)-1-(4-chlorobenzofuran-7-yl)-2-(2,6-dibromophenoxy)ethan-1-ol (320 mg, 0.72 mmol), 1,10-phenantroline (39 mg, 0.22 mmol), CuI (14 mg, 0.074 mmol) and Cs₂CO₃ (705 mg, 2.16 mmol) in toluene (5.0 mL) was stirred at 120° C. for 16 h. After the reaction was completed, the reaction was concentrated. The residue was purified by flash silica gel chromatography (12 g SepaFlash® Silica Flash Column, Eluent of 0~15% EtOAc/PE gradient@50 mL/min) to give (R)-5-bromo-2-(4-chlorobenzofuran-7-yl)-2,3-dihydrobenzo[b][1,4]dioxine (220 mg, 83.6% yield). LC-MS: m/z 365.0 (M+H)⁺.

### Step H tert-butyl (R)-4-(2-(4-chlorobenzofuran-7-yl)-2,3-dihydrobenzo[b][1,4]dioxin-5-yl)-3,6-dihydropyridine-1(2H)-carboxylate

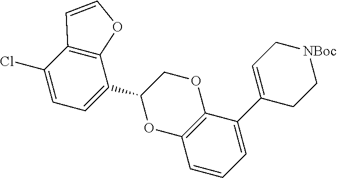 [SMILES: CC(C)(C)OC(=O)N1CC=C(c2cccc3c2OC[C@@H](c2ccc(Cl)c4ccoc24)O3)CC1]

To the mixture of (R)-5-bromo-2-(4-chlorobenzofuran-7-yl)-2,3-dihydrobenzo[b][1,4]dioxine (220 mg, 0.60 mmol), tert-butyl 4-(4,4,5,5-tetramethyl-1,3,2-dioxaborolan-2-yl)-3,6-dihydropyridine-1(2H)-carboxylate (280 mg, 0.91 mmol) and K₂CO₃ (250 mg, 1.81 mmol) in dioxane (5 mL) and H₂O (0.5 mL), was added Pd(dppf)Cl₂ (49 mg, 0.006 mmol). The reaction mixture was stirred at 90° C. for 16 h. After cooling, the mixture was diluted with H₂O (30 mL), extracted with EA (30 mL×2). The organic layer was combined and washed with brine (20 mL×2), dried over anhydrous Na₂SO₄, filtered and concentrated. The residue was purified by flash silica gel chromatography (12 g SepaFlash® Silica Flash Column, Eluent of 0~5% EtOAc/PE gradient@50 mL/min) to give tert-butyl (R)-4-(2-(4-chlorobenzofuran-7-yl)-2,3-dihydrobenzo[b][1,4]dioxin-5-yl)-3,6-dihydropyridine-1(2H)-carboxylate (210 mg, 74.5% yield). LC-MS: m/z 490.2 (M+Na)⁺.

### Step I tert-butyl (R)-4-(2-(4-chlorobenzofuran-7-yl)-2,3-dihydrobenzo[b][1,4]dioxin-5-yl)piperidine-1-carboxylate

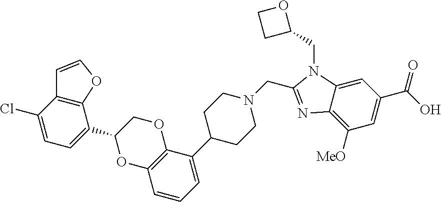 [SMILES: COc1cc(C(=O)O)cc2c1nc(CN1CCC(c3cccc4c3OC[C@@H](c3ccc(Cl)c5ccoc35)O4)CC1)n2C[C@@H]1CCO1]

To the mixture of tert-butyl (R)-4-(2-(4-chlorobenzofuran-7-yl)-2,3-dihydrobenzo[b][1,4]dioxin-5-yl)-3,6-dihydropyridine-1(2H)-carboxylate (175 mg, 0.37 mmol) in THF (3.0 mL) was added PtO₂ (8 mg, 0.037 mmol). The reaction mixture was degassed and charged with H₂ three times and then stirred at 25° C. for 1.5 h under H₂ balloon. After filtration, the filtrate was concentrated to give tert-butyl (R)-4-(2-(4-chlorobenzofuran-7-yl)-2,3-dihydrobenzo[b][1,4]dioxin-5-yl)piperidine-1-carboxylate (160 mg, crude). LC-MS: m/z 492.2 (M+Na)⁺.

### Step J (R)-4-(2-(4-chlorobenzofuran-7-yl)-2,3-dihydrobenzo[b][1,4]dioxin-5-yl)piperidine HCl salt

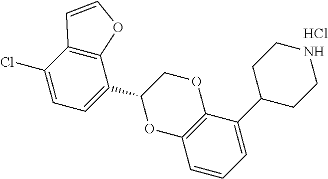 [SMILES: Clc1ccc([C@@H]2COc3c(cccc3C3CCNCC3)O2)c2occc12 ; Cl]

To a solution of tert-butyl (R)-4-(2-(4-chlorobenzofuran-7-yl)-2,3-dihydrobenzo[b][1,4]dioxin-5-yl)piperidine-1-carboxylate (80 mg, crude) in dioxane (2 mL) was added 4 M HCl (gas) in dioxane (1 mL). The reaction mixture was stirred at 25° C. for 1.5 h. then it was concentrated to get (R)-4-(2-(4-chlorobenzofuran-7-yl)-2,3-dihydrobenzo[b][1,4]dioxin-5-yl)piperidine HCl salt (80 mg, crude). LC-MS: m/z 370.1 (M+H)⁺.

### Step K 2-({4-[(2R)-2-(4-chloro-1-benzofuran-7-yl)-2,3-dihydro-1,4-benzodioxin-5-yl]piperidin-1-yl}methyl)-4-methoxy-1-{[(2S)-oxetan-2-yl]methyl}-1H-1,3-benzodiazole-6-carboxylic acid (Compound 120)

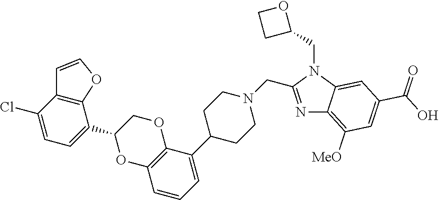 [SMILES: COc1cc(C(=O)O)cc2c1nc(CN1CCC(c3cccc4c3OC[C@@H](c3ccc(Cl)c5ccoc35)O4)CC1)n2C[C@@H]1CCO1]

2-({4-[(2R)-2-(4-chloro-1-benzofuran-7-yl)-2,3-dihydro-1,4-benzodioxin-5-yl]piperidin-1-yl}methyl)-4-methoxy-1-{([(2S)-oxetan-2-yl]methyl}-1H-1,3-benzodiazole-6-carboxylic acid (Compound 120) was synthesized according to the procedures described for the preparation of Example A5 (step A to B) by using (R)-4-(2-(4-chlorobenzofuran-7-yl)-2,3-dihydrobenzo[b][1,4]dioxin-5-yl)piperidine HCl salt and ethyl (S)-2-(chloromethyl)-4-methoxy-1-(oxetan-2-ylmethyl)-1H-benzo[d]imidazole-6-carboxylate in step A. LC-MS: m/z 644.3 (M+H)⁺. H NMR (400 MHz, DMSO-d6) δ 8.19 (d, J=2.0 Hz, 1H), 7.90 (s, 1H), 7.42 (s, 2H), 7.26 (s, 1H), 7.09 (d, J=2.0 Hz, 1H), 6.90-6.77 (m, 3H), 5.67 (dd, J₁=8.0 Hz, J₂=2.0 Hz, 1H), 5.15-5.02 (m, 1H), 4.82-4.72 (m, 1H), 4.68-4.57 (m, 2H), 4.53-4.45 (m, 1H), 4.40-4.29 (m, 2H), 3.95 (s, 3H), 3.91 (d, J=13.2 Hz, 1H), 3.77 (d, J=13.6 Hz, 1H), 3.05-2.95 (m, 1H), 2.93-2.82 (m, 2H), 2.76-2.65 (m, 1H), 2.47-2.36 (m, 1H), 2.30-2.09 (m, 2H), 1.82-1.74 (m, 1H), 1.72-1.55 (m, 3H).

### Example A7

### 2-({4-[(2R)-2-(4-chloro-1-benzofuran-7-yl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 122)

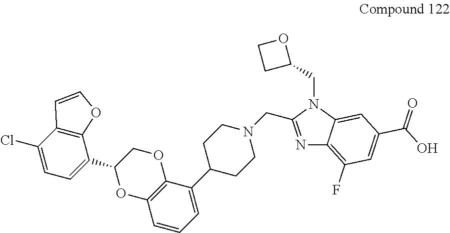 [SMILES: O=C(O)c1cc(F)c2nc(CN3CCC(c4cccc5c4OC[C@@H](c4ccc(Cl)c6ccoc46)O5)CC3)n(C[C@@H]3CCO3)c2c1]

2-({4-[(2R)-2-(4-chloro-1-benzofuran-7-yl)-2,3-dihydro-1,4-benzodioxin-5-yl]piperidin-1-yl}methyl)-4-fluoro-1-{[(2S)-oxetan-2-yl]methyl}-1H-1,3-benzodiazole-6-carboxylic acid (Compound 122) was synthesized according to the procedures described for the preparation of Example A5 (step A to B) by using (R)-4-(2-(4-chlorobenzofuran-7-yl)-2,3-dihydrobenzo[b][1,4]dioxin-5-yl)piperidine HCl salt and methyl (S)-2-(chloromethyl)-4-fluoro-1-(oxetan-2-ylmethyl)-1H-benzo[d]imidazole-6-carboxylate in step A. LC-MS: m/z 632.2 (M+H)⁺. ¹H NMR (400 MHz, DMSO-d6) δ 8.25-8.14 (m, 2H), 7.53 (d, J=11.6 Hz, 1H), 7.43 (s, 2H), 7.09 (d, J=2.0 Hz, 1H), 6.91-6.79 (m, 3H), 5.68 (dd, J₁=7.6 Hz, J₂=2.0 Hz, 1H), 5.17-5.05 (m, 1H), 4.91-4.80 (m, 1H), 4.72-4.68 (m, 1H), 4.64-4.60 (m, 1H), 4.56-4.46 (m, 1H), 4.43-4.30 (m, 2H), 4.10-3.72 (m, 2H), 3.10-2.82 (m, 3H), 2.79-2.66 (m, 1H), 2.43-2.36 (m, 1H), 2.36-2.08 (m, 2H), 1.88-1.58 (m, 4H). ¹⁹F NMR (377 MHz, DMSO-d6) δ−129.06.

Example compounds 115 and 116 were synthesized using a similar procedure described in the Example A7 above using the appropriate materials.

### Example A8

### 2-({4-[(2R)-2-(4-cyanophenyl)-2,3-dihydro-1,4-benzodioxin-5-yl]piperidin-1-yl}methyl)-4,7-difluoro-1-{[(2S)-oxetan-2-yl]methyl}-1H-1,3-benzodiazole-6-carboxylic acid (Compound 108)

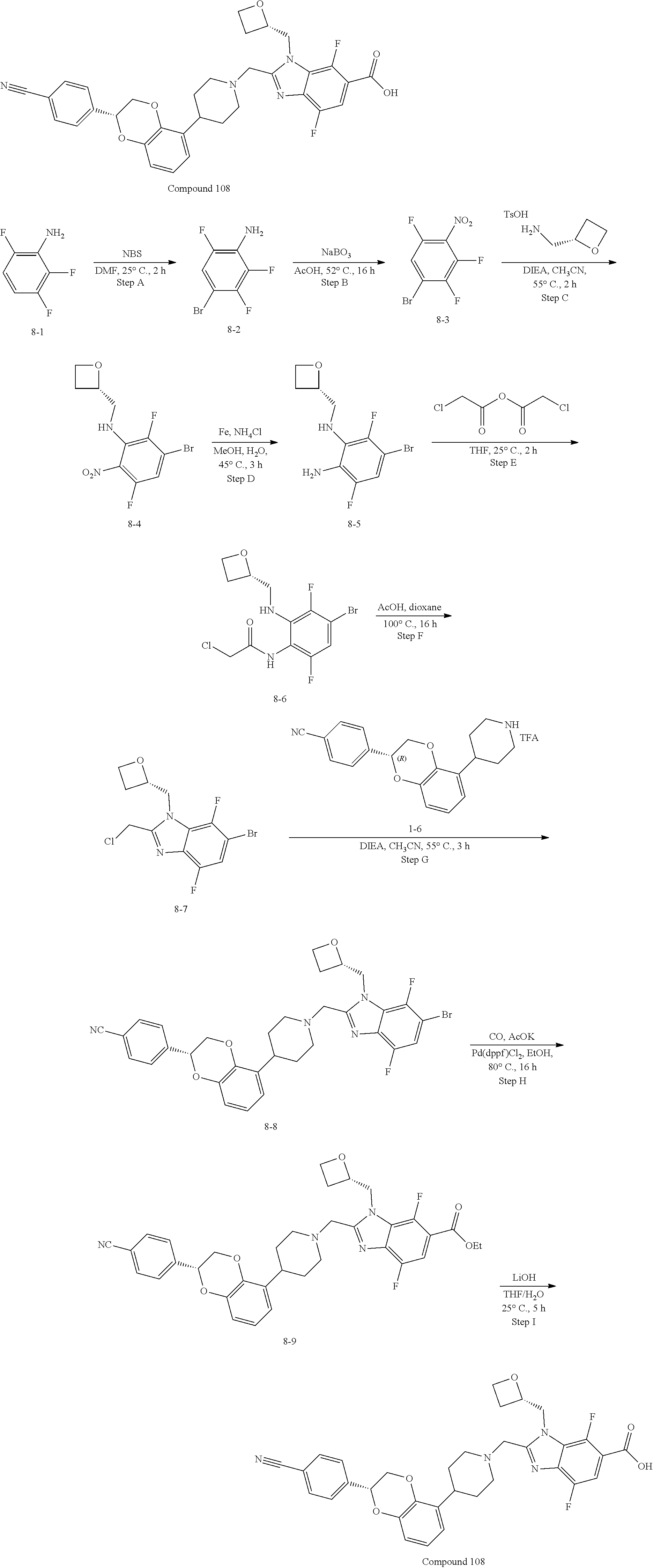

### Step A 4-bromo-2,3,6-trifluoroaniline

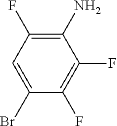 [SMILES: Nc1c(F)cc(Br)c(F)c1F]

The mixture of 2,3,6-trifluoroaniline (5.0 g, 34.00 mmol) and NBS (7.2 g, 40.80 mmol) in DMF (50 mL) was stirred at 25° C. for 2 h. Then the reaction mixture was diluted with EtOAc (200 mL), washed with 10% aq. Na₂SO₃ solution (100 mL×2), H₂O (100 mL) and brine (100 mL). The organic layer was dried over anhydrous Na₂SO₄, filtered and concentrated. The residue was purified by flash silica gel chromatography (120 g SepaFlash® Silica Flash Column, Eluent of 0~10% EtOAc/PE gradient @100 mL/min) to give 4-bromo-2,3,6-trifluoroaniline (6.0 g, 80% yield). ¹H NMR (400 MHz, DMSO-d6) δ 7.33 (ddd, J₁=10.4 Hz, J₂=6.0 Hz, J₃=2.4 Hz, 1H), 5.71 (s, 2H).

### Step B 1-bromo-2,3,5-trifluoro-4-nitrobenzene

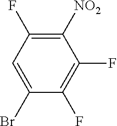 [SMILES: O=[N+]([O-])c1c(F)cc(Br)c(F)c1F]

The mixture of 4-bromo-2,3,6-trifluoroaniline (2.86 g, 12.71 mmol) and NaBO₃ (7.83 g, 50.84 mmol) in AcOH (50 mL) was stirred at 52° C. for 16 h. After cooling, the reaction mixture was concentrated. The residue was diluted with water and extracted with diethyl ether (100 mL×2). The combined organic extracts were washed with brine, dried over anhydrous Na₂SO₄, filtered and concentrated. The residue was purified by flash silica gel chromatography (120 g SepaFlash® Silica Flash Column, Eluent of 0~10% EtOAc/PE gradient@100 mL/min) to give 1-bromo-2,3,5-trifluoro-4-nitrobenzene (3.0 g, 93% yield).

### Step C (S)-3-bromo-2,5-difluoro-6-nitro-N-(oxetan-2-ylmethyl)aniline

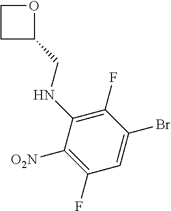 [SMILES: O=[N+]([O-])c1c(F)cc(Br)c(F)c1NC[C@@H]1CCO1]

The mixture of 1-bromo-2,3,5-trifluoro-4-nitrobenzene (800 mg, 3.14 mmol), (S)-oxetan-2-ylmethanamine TsOH salt (894 mg, 3.45 mmol) and DIEA (2.02 g, 15.70 mmol) in MeCN (15 mL) was stirred at 55° C. for 2 h. After cooling and concentration, the residue was purified by flash silica gel chromatography (40 g SepaFlash® Silica Flash Column, Eluent of 10-20% EtOAc/PE gradient@50 mL/min) to give (S)-3-bromo-2,5-difluoro-6-nitro-N-(oxetan-2-ylmethyl)aniline (340 mg, 34% yield). LC-MS: m/z 323.1 (M+H)⁺. ¹H NMR (400 MHz, DMSO-d6) δ 7.23 (dd, J₁=10.4 Hz, J₂=5.2 Hz, 1H), 6.91-6.87 (m, 1H), 4.87-4.81 (m, 1H), 4.51-4.46 (m, 1H), 4.33-4.27 (m, 1H), 3.54-3.46 (m, 2H), 2.64-2.55 (m, 1H), 2.46-2.37 (m, 1H).

### Step D (S)-5-bromo-3,6-difluoro-N1-(oxetan-2-ylmethyl)benzene-1,2-diamine

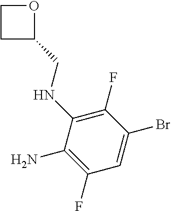 [SMILES: Nc1c(F)cc(Br)c(F)c1NC[C@@H]1CCO1]

To the mixture of (S)-3-bromo-2,5-difluoro-6-nitro-N-(oxetan-2-ylmethyl)aniline (340 mg, 1.06 mmol) and NH₄Cl (285 mg, 5.28 mmol) in EtOH/H₂O (20 mL/4 mL) was added Fe powder (296 mg, 5.28 mmol). The resulting mixture was stirred at 45° C. for 3 h. after cooling, the mixture was diluted with EtOAc (50 mL) and filtered. The filtrate was washed with brine (30 mL×2), dried over anhydrous Na₂SO₄, filtered and concentrated. The residue was purified by flash silica gel chromatography (24 g SepaFlash® Silica Flash Column, Eluent of 10-20% EtOAc/PE gradient@50 mL/min) to give (S)-5-bromo-3,6-difluoro-N1-(oxetan-2-ylmethyl)benzene-1,2-diamine (140 mg, 46% yield). LC-MS: m/z 295.1 (M+H)⁺.

### Step E (S)—N-(4-bromo-3,6-difluoro-2-((oxetan-2-ylmethyl)amino)phenyl)-2-chloroacetamide

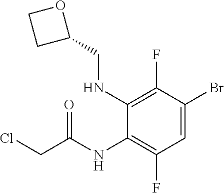 [SMILES: O=C(CCl)Nc1c(F)cc(Br)c(F)c1NC[C@@H]1CCO1]

To the mixture of (S)-5-bromo-3,6-difluoro-N1-(oxetan-2-ylmethyl)benzene-1,2-diamine (140 mg, 0.49 mmol) in THF (10 mL) was added 2-chloroacetic anhydride (91 mg, 0.54 mmol). The resulting mixture was stirred at 25° C. for 2 h. The reaction mixture was diluted with EtOAc (50 mL) and washed with sat.aq. NaHCO₃ (50 mL). The organic layer was dried over anhydrous Na₂SO₄, filtered and concentrated to give (S)—N-(4-bromo-3,6-difluoro-2-((oxetan-2-ylmethyl)amino)phenyl)-2-chloroacetamide (190 mg, crude). LC-MS: m/z 369.1 (M+H)⁺.

### Step F (S)-6-bromo-2-(chloromethyl)-4,7-difluoro-1-(oxetan-2-ylmethyl)-1H-benzo[d]imidazole

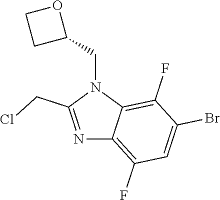 [SMILES: Fc1cc(Br)c(F)c2c1nc(CCl)n2C[C@@H]1CCO1]

The mixture of (S)—N-(4-bromo-3,6-difluoro-2-((oxetan-2-ylmethyl)amino)phenyl)-2-chloroacetamide (190 mg crude) in dioxane (10 mL)/AcOH (1 mL) was stirred at 100° C. for 16 h. After cooling, the reaction mixture was poured into sat.aq. Na₂CO₃ (20 mL), extracted with EtOAc (50 mL). The organic layer was washed with brine, dried over anhydrous Na₂SO₄, filtered and concentrated. The residue was purified by flash silica gel chromatography (12 g SepaFlash® Silica Flash Column, Eluent of 0~50% EtOAc/PE gradient@50 mL/min) to give (S)-6-bromo-2-(chloromethyl)-4,7-difluoro-1-(oxetan-2-ylmethyl)-1H-benzo[d]imidazole (170 mg, 93% yield). LC-MS: m/z 353.0 (M+H)⁺. ¹H NMR (400 MHz, DMSO-d6) δ 7.49 (dd, J₁=9.6 Hz, J₂=4.8 Hz, 1H), 5.15-5.03 (m, 3H), 4.77-4.71 (m, 1H), 4.63-4.59 (m, 1H), 4.51-4.46 (m, 1H), 4.38-4.32 (m, 1H), 2.77-2.69 (m, 1H), 2.43-2.36 (m, 1H). ¹⁹F NMR (377 MHz, DMSO-d6) δ−130.20,−131.30.

### Step G 4-((R)-5-(1-((6-bromo-4,7-difluoro-1-(((S)-oxetan-2-yl)methyl)-1H-benzo[d]imidazol-2-yl)methyl)piperidin-4-yl)-2,3-dihydrobenzo[b][1,4]dioxin-2-yl)benzonitrile

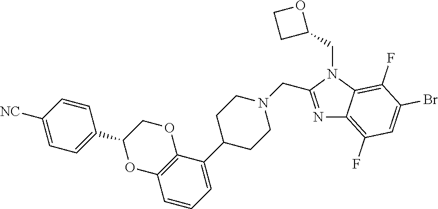 [SMILES: N#Cc1ccc([C@@H]2COc3c(cccc3C3CCN(Cc4nc5c(F)cc(Br)c(F)c5n4C[C@@H]4CCO4)CC3)O2)cc1]

The mixture of (S)-6-bromo-2-(chloromethyl)-4,7-difluoro-1-(oxetan-2-ylmethyl)-1H-benzo[d]imidazole (200 mg, 0.57 mmol), (R)-4-(5-(piperidin-4-yl)-2,3-dihydrobenzo[b][1,4]dioxin-2-yl)benzonitrile TFA salt (295 mg, 0.68 mmol) and DIEA (221 mg, 1.71 mmol) in MeCN (2 mL) was stirred at 55° C. for 3 h. After cooling and concentration, the residue was purified by flash silica gel chromatography (12 g SepaFlash® Silica Flash Column, Eluent of 0~5% MeOH/DCM gradient@50 mL/min) to give 4-((R)-5-(1-((6-bromo-4,7-difluoro-1-(((S)-oxetan-2-yl)methyl)-1H-benzo[d]imidazol-2-yl)methyl)piperidin-4-yl)-2,3-dihydrobenzo[b][1,4]dioxin-2-yl)benzonitrile (230 mg, 63.6% yield). LC-MS: m/z 635.2 (M+H)⁺.

### Step H ethyl 2-((4-((R)-2-(4-cyanophenyl)-2,3-dihydrobenzo[b][1,4]dioxin-5-yl)piperidin-1-yl)methyl)-4,7-difluoro-1-(((S)-oxetan-2-yl)methyl)-1H-benzo[d]imidazole-6-carboxylate

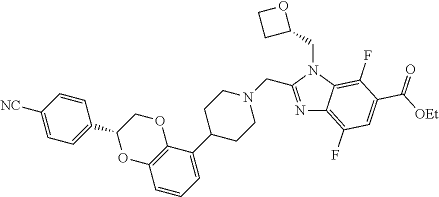 [SMILES: CCOC(=O)c1cc(F)c2nc(CN3CCC(c4cccc5c4OC[C@@H](c4ccc(C#N)cc4)O5)CC3)n(C[C@@H]3CCO3)c2c1F]

To a solution of 4-((R)-5-(1-((6-bromo-4,7-difluoro-1-(((S)-oxetan-2-yl)methyl)-1H-benzo[d]imidazol-2-yl)methyl)piperidin-4-yl)-2,3-dihydrobenzo[b][1,4]dioxin-2-yl)benzonitrile (120 mg, 0.19 mmol) in EtOH (5 mL) was added Pd(dppf)Cl₂·CH₂Cl₂ (15 mg, 0.019 mmol) and KOAc (56 mg, 0.57 mmol). The reaction mixture was stirred at 80° C. for 16 h under CO atmosphere. After the reaction was completed, the mixture was diluted with EtOAc (50 mL) and filtered, the filtrate was concentrated. The residue was purified by prep. TLC purification (DCM:MeOH=20:1) to give ethyl 2-((4-((R)-2-(4-cyanophenyl)-2,3-dihydrobenzo[b][1,4]dioxin-5-yl)piperidin-1-yl)methyl)-4,7-difluoro-1-(((S)-oxetan-2-yl)methyl)-1H-benzo[d]imidazole-6-carboxylate (90 mg, 75.7% yield). LC-MS: m/z 629.3 (M+H)⁺.

### Step I 2-({4-[(2R)-2-(4-cyanophenyl)-2,3-dihydro-1,4-benzodioxin-5-yl]piperidin-1-yl}methyl)-4,7-difluoro-1-{[(2S)-oxetan-2-yl]methyl}-1H-1,3-benzodiazole-6-carboxylic acid (Compound 108)

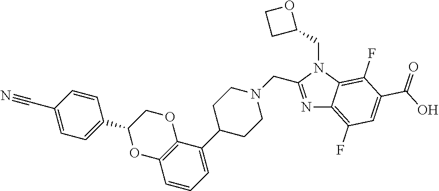 [SMILES: N#Cc1ccc([C@@H]2COc3c(cccc3C3CCN(Cc4nc5c(F)cc(C(=O)O)c(F)c5n4C[C@@H]4CCO4)CC3)O2)cc1]

To the mixture of ethyl 2-((4-((R)-2-(4-cyanophenyl)-2,3-dihydrobenzo[b][1,4]dioxin-5-yl)piperidin-1-yl)methyl)-4,7-difluoro-1-(((S)-oxetan-2-yl)methyl)-1H-benzo[d]imidazole-6-carboxylate (90 mg, 0.14 mmol) in THF (2 mL)/H₂O (2 mL) was added LiOH (17 mg, 0.72 mmol). The resulting mixture was stirred at 25° C. for 5 h. After concentration, the residue was purified by prep. HPLC purification (Column: Phenomenex C18, 19*250 mm*10 μm; Mobile Phase A: Water (0.1% formic acid), B: CH₃CN; Flow rate: 40 mL/min; Gradient: 20% B to 60% B; Retention Time: 10 min) to give 2-({4-[(2R)-2-(4-cyanophenyl)-2,3-dihydro-1,4-benzodioxin-5-yl]piperidin-1-yl}methyl)-4,7-difluoro-1-{[(2S)-oxetan-2-yl]methyl}-1H-1,3-benzodiazole-6-carboxylic acid (10.74 mg, 12.5% yield). LC-MS: m/z 601.3 (M+H)⁺. ¹H NMR (400 MHz, DMSO-d6) δ 7.91 (d, J=7.6 Hz, 2H), 7.69 (d, J=7.2 Hz, 2H), 7.38 (d, J=6.8 Hz, 11H), 6.85-6.80 (m, 3H), 5.38 (d, J=6.8 Hz, 1H), 5.11 (s, 11H), 4.94-4.89 (m, 1H), 4.76-4.72 (m, 1H), 4.56-4.50 (m, 2H), 4.38 (d, J=6.4 Hz, 1H), 4.12-4.07 (m, 1H), 3.97-3.83 (m, 2H), 3.02-2.99 (m, 1H), 2.92-2.89 (m, 2H), 2.76-2.74 (m, 1H), 2.46-2.44 (m, 1H), 2.29-2.23 (m, 2H), 1.75-1.66 (m, 4H). ¹⁹F NMR (377 MHz, DMSO-d6) δ −133.04,−133.52.

### Example A9

### 2-({4-[(2R)-2-(4-cyanophenyl)-2,3-dihydro-1,4-benzodioxin-5-yl]piperidin-1-yl}methyl)-1-[(3S,4S)-4-methoxyoxolan-3-yl]-1H-1,3-benzodiazole-6-carboxylic acid (Compound 114)

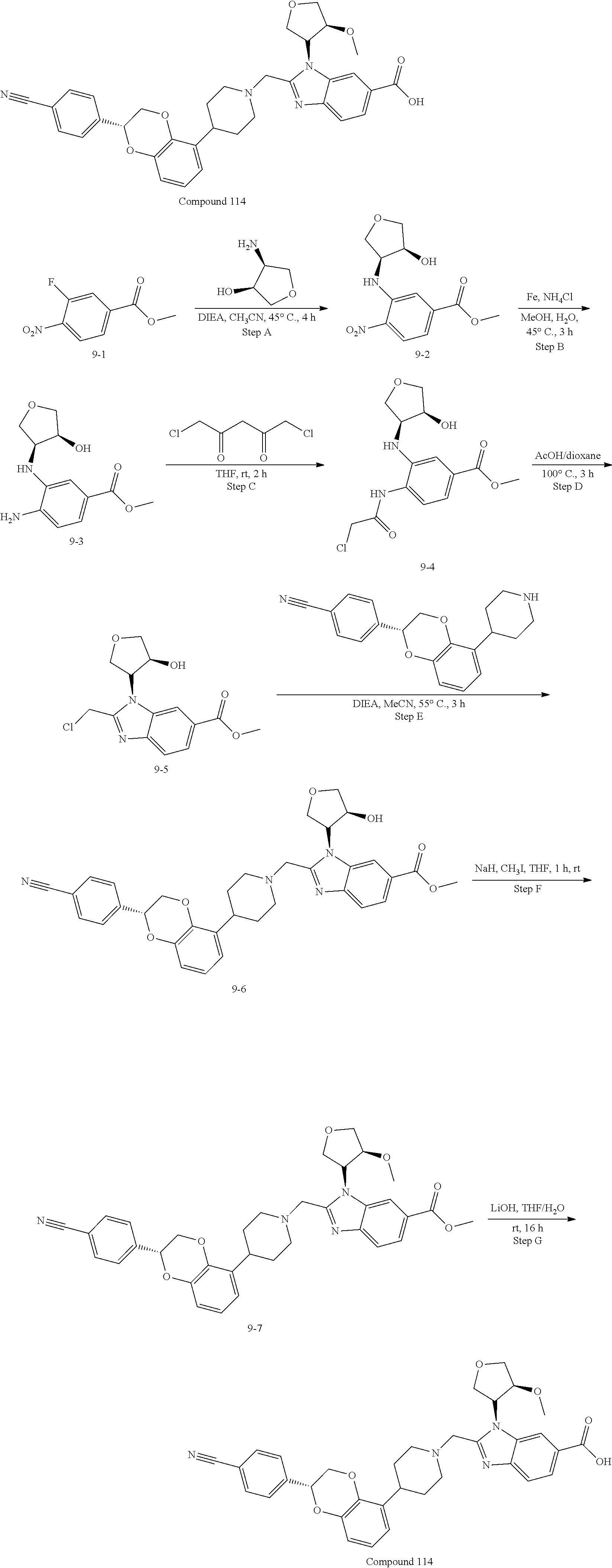

### Step A methyl 3-(((3S,4S)-4-hydroxytetrahydrofuran-3-yl)amino)-4-nitrobenzoate

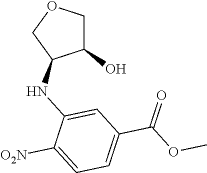 [SMILES: COC(=O)c1ccc([N+](=O)[O-])c(N[C@H]2COC[C@H]2O)c1]

The mixture of methyl 3-fluoro-4-nitrobenzoate (117 mg, 0.59 mmol), (3S,4S)-4-aminotetrahydrofuran-3-ol (200 mg, 0.71 mmol), DIEA (228 mg, 1.77 mmol) in CH₃CN (20 mL) was stirred at 45° C. for 4 h. The resulted mixture were concentrated and then purified by flash silica gel chromatography (12 g SepaFlash® Silica Flash Column, Eluent of 0~20% EtOAc/PE gradient@50 mL/min) to give methyl 3-(((3S,4S)-4-hydroxytetrahydrofuran-3-yl)amino)-4-nitrobenzoate (140 mg, 83.3% yield). LC-MS: m/z 283.2 (M+H)⁺.

### Step B methyl 4-amino-3-(((3S,4S)-4-hydroxytetrahydrofuran-3-yl)amino)benzoate

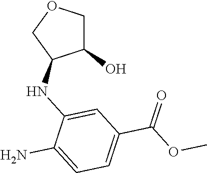 [SMILES: COC(=O)c1ccc(N)c(N[C@H]2COC[C@H]2O)c1]

The mixture of methyl 3-(((3S,4S)-4-hydroxytetrahydrofuran-3-yl)amino)-4-nitrobenzoate (140 mg, 0.50 mmol), Fe powder (84 mg, 1.5 mmol), NH₄Cl (80 mg, 1.5 mmol) in EtOH/H₂O (10 mL/2 mL) was stirred at 45° C. for 3 h. The mixture was diluted with EtOAc (50 mL), filtered and the filtrate was washed with brine (50 mL×3). The organic layer was separated and dried over anhydrous Na₂SO₄, filtered and concentrated, The residue was purified by flash silica gel chromatography (12 g SepaFlash® Silica Flash Column, Eluent of 0~50% EtOAc/PE gradient@50 mL/min) to give methyl 4-amino-3-(((3S,4S)-4-hydroxytetrahydrofuran-3-yl)amino)benzoate (100 mg, 80% yield). LC-MS: m/z 253.2 (M+H)⁺.

### Step C methyl 4-(2-chloroacetamido)-3-(((3S,4S)-4-hydroxytetrahydrofuran-3-yl)amino)benzoate

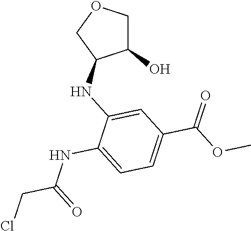 [SMILES: COC(=O)c1ccc(NC(=O)CCl)c(N[C@H]2COC[C@H]2O)c1]

The mixture of methyl 4-amino-3-(((3S,4S)-4-hydroxytetrahydrofuran-3-yl)amino)benzoate (100 mg, 0.40 mmol) and 2-chloroacetic anhydride (74 mg, 0.44 mmol) in THF (5 mL) was stirred at room temperature for 2 h. The mixture was poured into sat. aq. Na₂CO₃, extracted with EtOAc (100 mL). The organic layer was separated and dried over anhydrous Na₂SO₄, filtered and concentrated, The residue was purified by flash silica gel chromatography (12 g SepaFlash® Silica Flash Column, Eluent of 0~50% EtOAc/PE gradient@50 mL/min) to give methyl 4-(2-chloroacetamido)-3-(((3S,4S)-4-hydroxytetrahydrofuran-3-yl)amino)benzoate (120 mg, 92.3% yield). LC-MS: m/z 329.2 (M+H)⁺.

### Step D methyl 2-(chloromethyl)-1-((3S,4S)-4-hydroxytetrahydrofuran-3-yl)-1H-benzo[d]imidazole-6-carboxylate

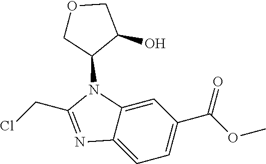 [SMILES: COC(=O)c1ccc2nc(CCl)n([C@H]3COC[C@H]3O)c2c1]

The mixture of methyl 4-(2-chloroacetamido)-3-(((3S,4S)-4-hydroxytetrahydrofuran-3-yl)amino)benzoate (120 mg, 0.37 mmol) in AcOH/dioxane (1 mL/10 mL) was stirred at 100° C. for 3 h. After cooling, the mixture was poured into sat.aq. Na₂CO₃ and extracted with EtOAc (100 mL). The organic layer was separated and dried over anhydrous Na₂SO₄, filtered and concentrated, the residue was purified by flash silica gel chromatography (12 g SepaFlash® Silica Flash Column, Eluent of 0~50% EtOAc/PE gradient@50 mL/min) to give methyl 2-(chloromethyl)-1-((3S,4S)-4-hydroxytetrahydrofuran-3-yl)-1H-benzo[d]imidazole-6-carboxylate (60 mg, 53.1% yield). LC-MS: m/z 311.1 (M+H)⁺.

### Step E methyl 2-((4-((R)-2-(4-cyanophenyl)-2,3-dihydrobenzo[b][1,4]dioxin-5-yl)piperidin-1-yl)methyl)-1-((3S,4S)-4-hydroxytetrahydrofuran-3-yl)-1H-benzo[d]imidazole-6-carboxylate

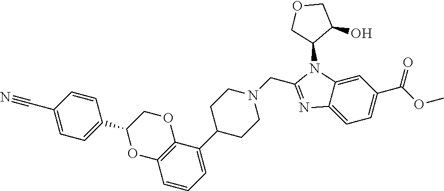 [SMILES: COC(=O)c1ccc2nc(CN3CCC(c4cccc5c4OC[C@@H](c4ccc(C#N)cc4)O5)CC3)n([C@H]3COC[C@H]3O)c2c1]

The mixture of methyl 2-(chloromethyl)-1-((3S,4S)-4-hydroxytetrahydrofuran-3-yl)-1H-benzo[d]imidazole-6-carboxylate (60 mg, 0.20 mmol), (R)-4-(5-(piperidin-4-yl)-2,3-dihydrobenzo[b][1,4]dioxin-2-yl)benzonitrile TFA salt (100 mg, 0.23 mmol) and DIEA (129 mg, 1.00 mmol) in CH₃CN (10 mL) was stirred at 55° C. for 3 h. The mixture was concentrated and the residue was purified by flash silica gel chromatography (12 g SepaFlash® Silica Flash Column, Eluent of 0~50% EtOAc/PE gradient@50 mL/min) to give methyl 2-((4-((R)-2-(4-cyanophenyl)-2,3-dihydrobenzo[b][1,4]dioxin-5-yl)piperidin-1-yl)methyl)-1-((3S,4S)-4-hydroxytetrahydrofuran-3-yl)-1H-benzo[d]imidazole-6-carboxylate (110 mg, 95.7% yield). LC-MS: m/z 595.3 (M+H)⁺.

### Step F methyl 2-((4-((R)-2-(4-cyanophenyl)-2,3-dihydrobenzo[b][1,4]dioxin-5-yl)piperidin-1-yl)methyl)-1-((3S,4S)-4-methoxytetrahydrofuran-3-yl)-1H-benzo[d]imidazole-6-carboxylate

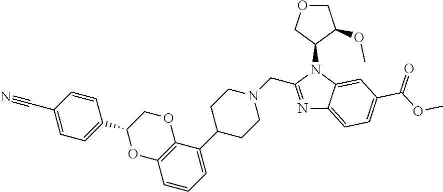 [SMILES: COC(=O)c1ccc2nc(CN3CCC(c4cccc5c4OC[C@@H](c4ccc(C#N)cc4)O5)CC3)n([C@H]3COC[C@H]3OC)c2c1]

The mixture of methyl 2-((4-((R)-2-(4-cyanophenyl)-2,3-dihydrobenzo[b][1,4]dioxin-5-yl)piperidin-1-yl)methyl)-1-((3S,4S)-4-hydroxytetrahydrofuran-3-yl)-1H-benzo[d]imidazole-6-carboxylate (110 mg, 0.19 mmol) and NaH (18 mg, 0.74 mmol), and CH₃I (105 mg, 0.74 mmol) in THF (10 mL) was stirred at room temperature for 1 h. After the reaction was completed, the mixture was diluted with EtOAc (50 mL), washed with brine (50 mL×2), dried, filtered, concentrated to give methyl 2-((4-((R)-2-(4-cyanophenyl)-2,3-dihydrobenzo[b][1,4]dioxin-5-yl)piperidin-1-yl)methyl)-1-((3S,4S)-4-methoxytetrahydrofuran-3-yl)-1H-benzo[d]imidazole-6-carboxylate (150 mg, crude). LC-MS: m/z 609.4 (M+H)⁺.

### Step G 2-({4-[(2R)-2-(4-cyanophenyl)-2,3-dihydro-1,4-benzodioxin-5-yl]piperidin-1-yl}methyl)-1-[(3S,4S)-4-methoxyoxolan-3-yl]-1H-1,3-benzodiazole-6-carboxylic acid (Compound 114)

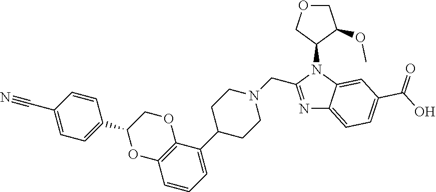 [SMILES: CO[C@@H]1COC[C@@H]1n1c(CN2CCC(c3cccc4c3OC[C@@H](c3ccc(C#N)cc3)O4)CC2)nc2ccc(C(=O)O)cc21]

The mixture of methyl 2-((4-((R)-2-(4-cyanophenyl)-2,3-dihydrobenzo[b][1,4]dioxin-5-yl)piperidin-1-yl)methyl)-1-((3S,4S)-4-methoxytetrahydrofuran-3-yl)-1H-benzo[d]imidazole-6-carboxylate (75 mg, 0.12 mmol) and LiOH (26 mg, 0.61 mmol) in H₂O (4 mL)/THF (12 mL) was stirred at rt for 16 h. After the reaction was completed, the reaction was diluted with H₂O (20 mL), adjusted with HCl (1 M) until pH=5-6, followed the mixture was extracted with EtOAc (20 mL×2). The organic layer was washed with brine, dried over anhydrous Na₂SO₄, filtered and concentrated under vacuum to dryness. The crude was purified by prep. HPLC purification (Column: Sunfire C18, 19*250 mm*10 m; Mobile Phase A: Water (0.1% formic acid), B: CH₃CN; Flow rate: 20 mL/min; Gradient: 28% B to 30% B; Retention Time: 8.8-10.1 min of 15 min) to give 2-({4-[(2R)-2-(4-cyanophenyl)-2,3-dihydro-1,4-benzodioxin-5-yl]piperidin-1-yl}methyl)-1-[(3S,4S)-4-methoxyoxolan-3-yl]-1H-1,3-benzodiazole-6-carboxylic acid (7.03 mg, 9.6% yield). LC-MS: m/z 595.4 (M+H)⁺. ¹H NMR (400 MHz, DMSO-d6) δ 12.68 (s, 1H), 8.37 (s, 1H), 7.92 (d, J=8.0 Hz, 2H), 7.77 (d, J=8.4 Hz, 1H), 7.69 (d, J=8.0 Hz, 2H), 7.61 (d, J=8.4 Hz, 1H), 6.85-6.81 (m, 3H), 5.63-5.56 (m, 1H), 5.38-5.36 (m, 1H), 4.56-4.53 (m, 1H), 4.37-4.30 (m, 2H), 4.16-4.05 (m, 3H), 4.01 (t, J=8.8 Hz, 1H), 3.87 (dd, J₁=10.4 Hz, J₂=4.0 Hz, 1H), 3.67 (d, J=13.2 Hz, 1H), 3.08-3.05 (m, 1H), 2.89 (s, 3H), 2.86-2.82 (m, 2H), 2.33-2.21 (m, 2H), 2.15-2.09 (m, 1H), 1.71-1.69 (m, 3H).

### Example A10

### 2-({6-[(2R)-2-(4-cyanophenyl)-2,3-dihydro-1,4-benzodioxin-5-yl]-3-azabicyclo[4.1.0]heptan-3-yl}methyl)-1-{[(2S)-oxetan-2-yl]methyl}-1H-1,3-benzodiazole-6-carboxylic acid (Compound 110)

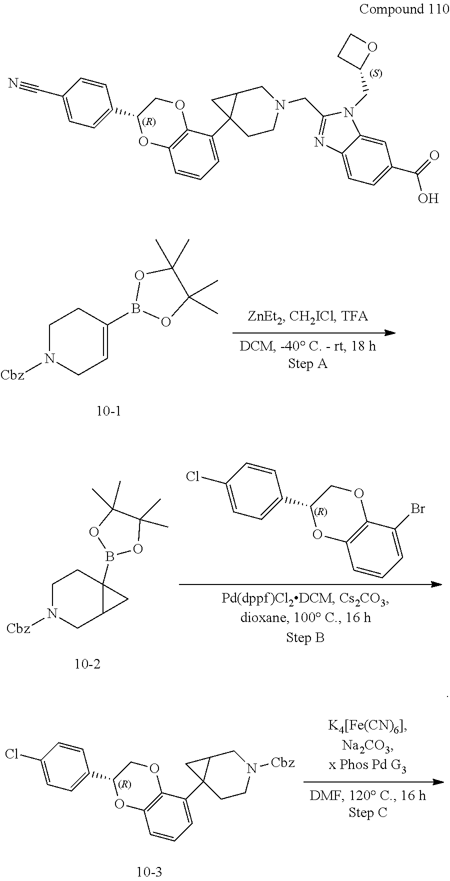

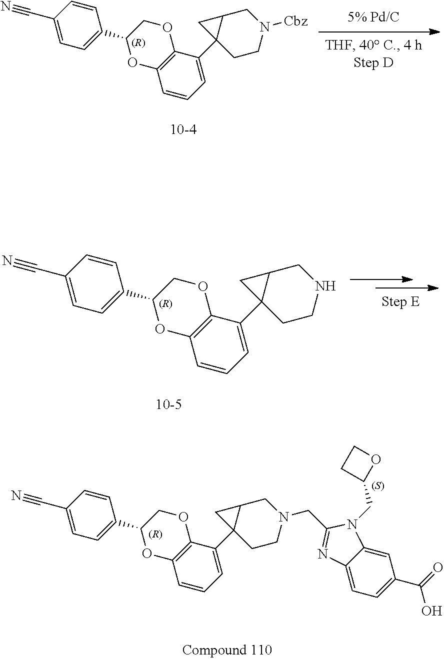

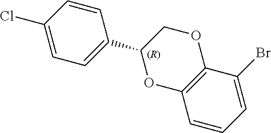

(R)-5-bromo-2-(4-chlorophenyl)-2,3-dihydrobenzo[b][1,4]dioxine was synthesized according to the procedures described for the preparation of intermediate 6-8 (step E, F and G) by using 2,6-dibromophenol and 2-bromo-1-(4-chlorophenyl)ethan-1-one in step E. ¹H NMR (400 MHz, DMSO-d6) δ 7.41 (dd, J₁=8.0 Hz, J₂=2.0 Hz, 2H), 7.36 (d, J=8.4 Hz, 2H), (dd, J=8.0 Hz, 1.2 Hz, 1H), 6.95-6.93 (m, 1H), 6.78 (t, J=8.0 Hz, 1H), 5.11 (dd, J₁=8.8 Hz, J₂=2.4 Hz, 1H), 4.48 (dd, J₁=10.6 Hz, J₂=2.4 Hz, 1H), 4.05 (dd, J₁=11.6 Hz, J₂=8.8 Hz, 1H).

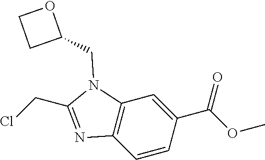 [SMILES: COC(=O)c1ccc2nc(CCl)n(C[C@@H]3CCO3)c2c1]

methyl (S)-2-(chloromethyl)-1-(oxetan-2-ylmethyl)-1H-benzo[d]imidazole-6-carboxylate was synthesized according to the procedures described for the preparation of intermediate 1-4 (step A, B and C) by using methyl 3-fluoro-4-nitrobenzoate and (2S)-oxetan-2-ylmethanamine in step A.

LC-MS: m/z 295.1 (M+H)⁺.

### Step A benzyl 6-(4,4,5,5-tetramethyl-1,3,2-dioxaborolan-2-yl)-3-azabicyclo[4.1.0]heptane-3-carboxylate

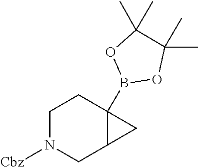 [SMILES: CC1(C)OB(C23CCN(C(=O)OCc4ccccc4)CC2C3)OC1(C)C]

To a solution of ZnEt₂ (1 M THF solution, 69.93 mL, 69.93 mmol) in DCM (45 mL) was added CH₂ClI (24.7 g, 139.94 mmol) slowly at −40° C. and the mixture was stirred at −40° C. for 1 h. TFA (7.98 g, 69.93 mmol) was added 40° C. and the mixture was stirred at −15° C. for another 1 h. Then benzyl 4-(4,4,5,5-tetramethyl-1,3,2-dioxaborolan-2-yl)-3,6-dihydropyridine-1(2H)-carboxylate (3 g, 8.73 mmol) in DCM (15 mL) was added to the reaction mixture slowly at −15° C. after addition, the reaction mixture was stirred at 25° C. for 16 h. After the reaction was complete, the mixture was concentrated and purified by flash silica gel chromatography (120 g SepaFlash® Silica Flash Column, Eluent of 0~20% EtOAc/PE gradient@100 mL/min) to give benzyl 6-(4,4,5,5-tetramethyl-1,3,2-dioxaborolan-2-yl)-3-azabicyclo[4.1.0]heptane-3-carboxylate (1.8 g, 57.6% yield). LC-MS: m/z 358.2 (M+H)⁺. ¹H NMR (400 MHz, DMSO-d6) δ 7.38-7.29 (m, 5H), 5.09-5.05 (m, 2H), 3.76-3.73 (m, 1H), 3.52 (s, 1H), 3.43-3.37 (m, 1H), 2.89-2.86 (m, 1H), 2.02-1.96 (m, 1H), 1.50-1.42 (m, 1H), 1.23-1.18 (m, 13H), 0.78-0.75 (m, 1H), 0.37-0.35 (m, 1H).

### Step B benzyl 6-((R)-2-(4-chlorophenyl)-2,3-dihydrobenzo[b][1,4]dioxin-5-yl)-3-azabicyclo[4.1.0]heptane-3-carboxylate

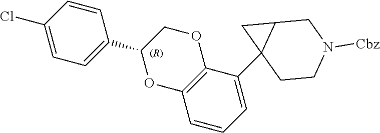

A mixture of (R)-5-bromo-2-(4-chlorophenyl)-2,3-dihydrobenzo[b][1,4]dioxine (400 mg, 1.23 mmol), benzyl 6-(4,4,5,5-tetramethyl-1,3,2-dioxaborolan-2-yl)-3-azabicyclo[4.1.0]heptane-3-carboxylate (527 mg, 1.47 mmol), Cs₂CO₃ (1.2 g, 3.69 mmol) and Pd(dppf)Cl₂·DCM (98 mg, 0.12 mmol) in dioxane (10 mL) was stirred at 100° C. for 16 h. After the reaction was completed, the mixture was concentrated and purified by reverse ISCO (CH₃CN/Water (0.1% formic acid)=0 to 85.2%) to give benzyl 6-((R)-2-(4-chlorophenyl)-2,3-dihydrobenzo[b][1,4]dioxin-5-yl)-3-azabicyclo[4.1.0]heptane-3-carboxylate (250 mg, 42.8% yield). LC-MS: m/z 476.2 (M+H)⁺.

### Step C benzyl 6-((R)-2-(4-cyanophenyl)-2,3-dihydrobenzo[b][1,4]dioxin-5-yl)-3-azabicyclo[4.1.0]heptane-3-carboxylate

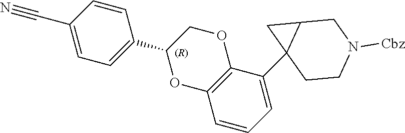

A mixture of benzyl 6-((R)-2-(4-chlorophenyl)-2,3-dihydrobenzo[b][1,4]dioxin-5-yl)-3-azabicyclo[4.1.0]heptane-3-carboxylate (250 mg, 0.53 mmol), K₄[Fe(CN)₆](967 mg, 2.63 mmol), Na₂CO₃ (167 mg, 1.58 mmol) and Xphos Pd G3 (43 mg, 0.05 mmol) in DMF (10 mL) was stirred at 120° C. for 16 h. After the reaction was completed, the mixture was concentrated and purified by reversed ISCO (CH₃CN/Water (0.1% formic acid)=0 to 82%) to give benzyl 6-((R)-2-(4-cyanophenyl)-2,3-dihydrobenzo[b][1,4]dioxin-5-yl)-3-azabicyclo[4.1.0]heptane-3-carboxylate (150 mg, 61.2% yield). LC-MS: m/z 467.2 (M+H)⁺.

### Step D 4-((2R)-5-(3-azabicyclo[4.1.0]heptan-6-yl)-2,3-dihydrobenzo[b][1,4]dioxin-2-yl)benzonitrile

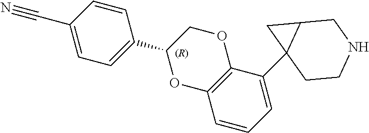

The mixture of benzyl 6-((R)-2-(4-cyanophenyl)-2,3-dihydrobenzo[b][1,4]dioxin-5-yl)-3-azabicyclo[4.1.0]heptane-3-carboxylate (150 mg, 0.32 mmol) and 5% Pd/C (15 mg) in THF (10 mL) was stirred at 40° C. for 4 h under H₂ balloon. After filtration, the filtrate was concentrated to give 4-((2R)-5-(3-azabicyclo[4.1.0]heptan-6-yl)-2,3-dihydrobenzo[b][1,4]dioxin-2-yl)benzonitrile (60 mg, 56.14% yield). LC-MS: m/z 333.2 (M+H)⁺.

### Step E 2-({6-[(2R)-2-(4-cyanophenyl)-2,3-dihydro-1,4-benzodioxin-5-yl]-3-azabicyclo[4.1.0]heptan-3-yl}methyl)-1-{[(2S)-oxetan-2-yl]methyl}-1H-1,3-benzodiazole-6-carboxylic acid (Compound 110)

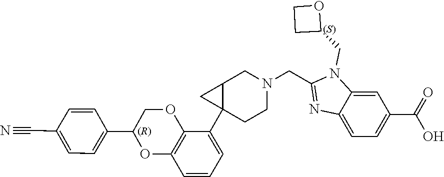

2-({6-[(2R)-2-(4-cyanophenyl)-2,3-dihydro-1,4-benzodioxin-5-yl]-3-azabicyclo[4.1.0]heptan-3-yl}methyl)-1-{[(2S)-oxetan-2-yl]methyl}-1H-1,3-benzodiazole-6-carboxylic acid (Compound 110) was synthesized according to the procedures described for the preparation of A1 (step E and F) by using 4-((2R)-5-(3-azabicyclo[4.1.0]heptan-6-yl)-2,3-dihydrobenzo[b][1,4]dioxin-2-yl)benzonitrile and methyl (S)-2-(chloromethyl)-1-(oxetan-2-ylmethyl)-1H-benzo[d]imidazole-6-carboxylate in step E. LC-MS: m/z 358.2 (M+H)⁺. ¹H NMR (400 MHz, DMSO-d6) δ 8.25 (s, 1H), 7.92 (d, J=8.0 Hz, 2H), 7.79-7.81 (m, 1H), 7.71 (d, J=8.0 Hz, 2H), 7.63-7.60 (m, 1H), 6.85-6.83 (m, 1H), 6.78-6.76 (m, 2H), 5.40-5.38 (m, 1H), 5.09-5.08 (m, 1H), 4.84-4.81 (m, 1H), 4.71-4.66 (m, 1H), 4.59-4.57 (m, 1H), 4.49-4.46 (m, 1H), 4.42-4.34 (m, 1H), 4.15-4.10 (m, 1H), 3.96-3.81 (m, 2H), 3.65 (d, J=13.2 Hz, 1H), 2.92-2.82 (m, 1H), 2.77-2.68 (m, 2H), 2.46-2.33 (m, 2H), 2.26-2.20 (m, 1H), 2.00-1.90 (m, 1H), 1.23-1.21 (m, 1H), 0.91-0.78 (m, 2H).

Example compounds 125, 126 and 127 were synthesized using a similar procedure described in the Example A10 above using the appropriate materials.

### Example A11

### 2-((4-((R)-3-(4-chlorophenyl)-2,3-dihydro-[1,4]dioxino[2,3-b]pyridin-8-yl)piperidin-1-yl)methyl)-1-(((S)-oxetan-2-yl)methyl)-1H-benzo[d]imidazole-6-carboxylic acid (Compound 130)

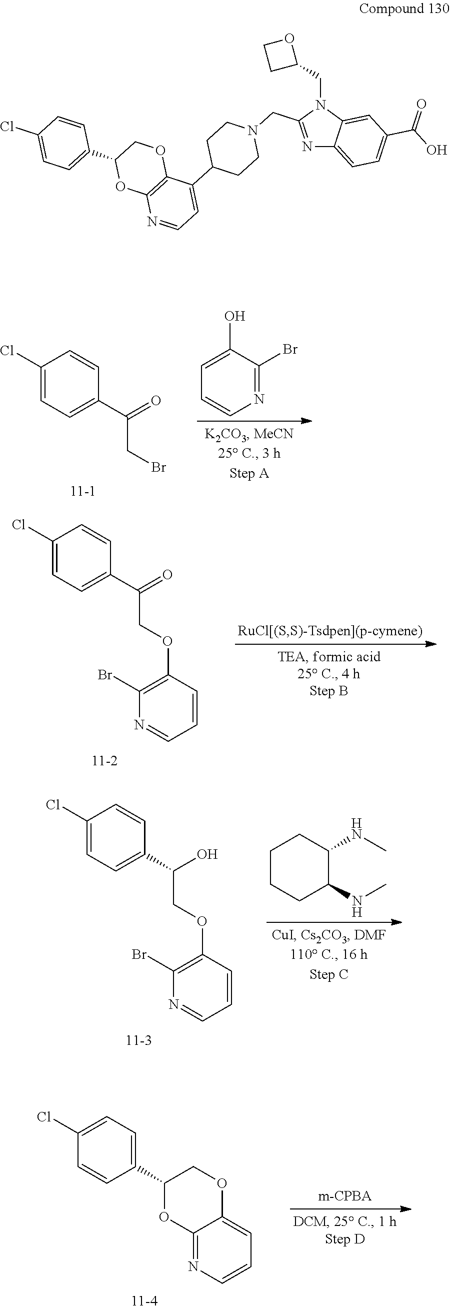

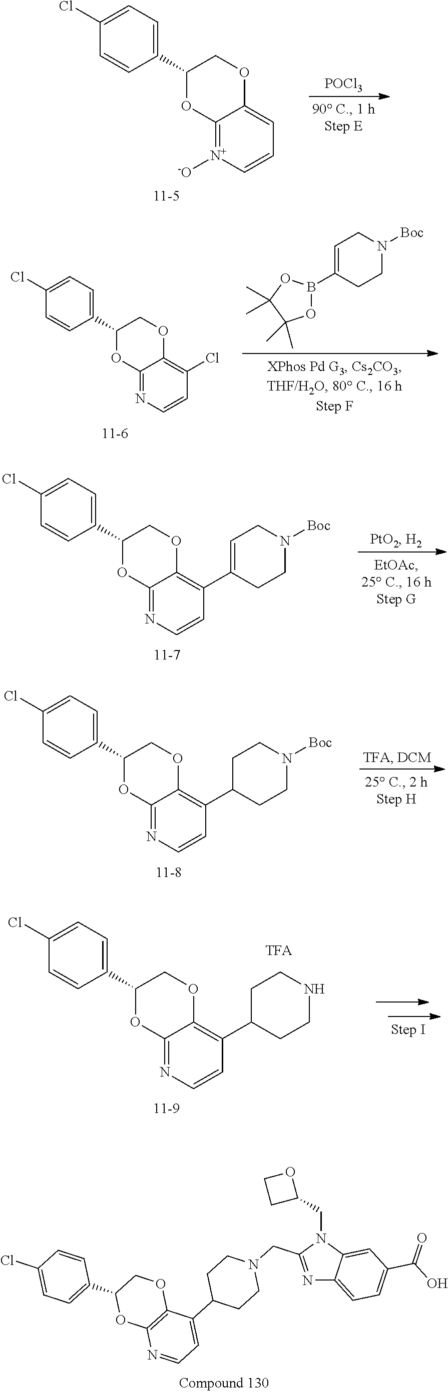

### Step A 2-((2-bromopyridin-3-yl)oxy)-1-(4-chlorophenyl)ethan-1-one

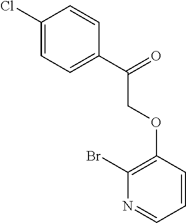 [SMILES: O=C(COc1cccnc1Br)c1ccc(Cl)cc1]

To a solution of 2-bromopyridin-3-ol (10 g, 57.5 mmol) and 2-bromo-1-(4-chlorophenyl)ethan-1-one (13.4 g, 57.5 mmol) in MeCN (300 mL) was added K₂CO₃ (15.9 g, 115 mmol). The mixture was stirred at 25° C. for 3 h. The mixture was diluted with H₂O (100 mL), the precipitate was collected by filtration. The solid was purified by flash silica gel chromatography (Silica Flash Column, Eluent of 50-60% EtOAc/PE gradient) to give 2-((2-bromopyridin-3-yl)oxy)-1-(4-chlorophenyl)ethan-1-one (17.5 g, 93.2% yield). ¹H NMR (400 MHz, CDCl₃) δ 8.04 (d, J=4.0 Hz, 1H), 7.98 (d, J=8.4 Hz, 2H), 7.50 (d, J=8.4 Hz, 2H), 7.19 (dd, J₁=8.0 Hz, J₂=4.7 Hz, 1H), 7.08 (d, J=8.0 Hz, 1H), 5.33 (s, 2H)

### Step B (S)-2-((2-bromopyridin-3-yl)oxy)-1-(4-chlorophenyl)ethan-1-ol

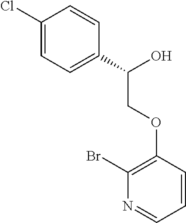 [SMILES: O[C@H](COc1cccnc1Br)c1ccc(Cl)cc1]

To a mixture of formic acid (35 mL) and TEA (175 mL) was added RuCl[(S,S)-Tsdpen](p-cymene) (1.71 g, 2.68 mmol). The mixture was stirred at 25° C. for 1 h. then 2-[(2-bromo-3-pyridyl)oxy]-1-(4-chlorophenyl)ethanone (17.5 g, 53.6 mmol) was added, the resulting mixture was stirred at 25° C. for 4 h. The reaction mixture was diluted with H₂O (100 mL) and extracted with EtOAc (100 mL×2). The combined organic phase was dried over Na₂SO₄, filtered and concentrated. The residue was purified by flash silica gel chromatography (Silica Flash Column, Eluent of 30-50% EtOAc/PE gradient) to give (S)-2-((2-bromopyridin-3-yl)oxy)-1-(4-chlorophenyl)ethan-1-ol (15 g, 85.7% yield). ¹H NMR (400 MHz, CDCl3) δ 7.96 (d, J=4.2 Hz, 1H), 7.44-7.38 (m, 2H), 7.37-7.30 (m, 2H), 7.20-7.08 (m, 2H), 5.17 (d, J=4.6 Hz, 1H), 4.17-4.06 (m, 2H), 3.54 (s, 1H)

### Step C (R)-3-(4-chlorophenyl)-2,3-dihydro-[1,4]dioxino[2,3-b]pyridine

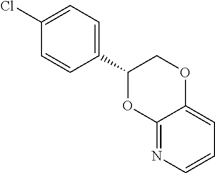 [SMILES: Clc1ccc([C@@H]2COc3cccnc3O2)cc1]

To a solution of (S)-2-((2-bromopyridin-3-yl)oxy)-1-(4-chlorophenyl)ethan-1-ol (8.5 g, 25.9 mmol) in DMF (20 mL) was added CuI (4.93 g, 25.9 mmol), (1 S,2S)—N1,N2-dimethylcyclohexane-1,2-diamine (7.36 g, 51.7 mmol) and Cs₂CO₃ (16.9 g, 51.7 mmol). The mixture was stirred at 110° C. for 16 h. After cooling, the reaction mixture was diluted with brine, extracted with EtOAc (100 mL×2). The combined organic layer was dried over Na₂SO₄, filtered and concentrated. The residue was purified by flash silica gel chromatography (Silica Flash Column, Eluent of 10~60% EtOAc/PE gradient) to give (R)-3-(4-chlorophenyl)-2,3-dihydro-[1,4]dioxino[2,3-b]pyridine (1.2 g, 18.7% yield). ¹H NMR (400 MHz, CDCl3) δ 7.89 (d, J=4.2 Hz, 1H), 7.46-7.36 (m, 4H), 7.28-7.23 (m, 1H), 6.93 (dd, J₁=7.8, J₂=4.8 Hz, 1H), 5.31 (dd, J=8.8, 2.4 Hz, 1H), 4.36 (dd, J=11.6, 2.4 Hz, 1H), 3.98 (dd, J=11.6, 8.8 Hz, 1H)

### Step D (R)-3-(4-chlorophenyl)-2,3-dihydro-[1,4]dioxino[2,3-b]pyridine 5-oxide

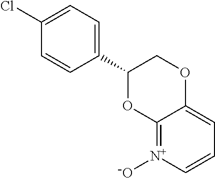 [SMILES: O[n+]1cccc2c1O[C@H](c1ccc(Cl)cc1)CO2]

To a solution of (3R)-3-(4-chlorophenyl)-2,3-dihydro-[1,4]dioxino[2,3-b]pyridine (150 mg, 605.63 μmol) in DCM (5 mL) was added 85% m-CPBA (620 mg, 727 μmol). The mixture was stirred at 25° C. for 1 h. The reaction mixture was diluted with sat. aq. NaHCO₃ (20 mL) and extracted with DCM (20 mL×2). The combined organic layer was dried over Na₂SO₄, filtered and concentrated to give (3R)-3-(4-chlorophenyl)-5-oxido-2,3-dihydro-[1,4]dioxino[2,3-b]pyridin-5-ium (145 mg, 90.7% yield), which used for next step without purification. LC-MS: m/z 264.2 (M+H)⁺.

### Step E (R)-8-chloro-3-(4-chlorophenyl)-2,3-dihydro-[1,4]dioxino[2,3-b]pyridine

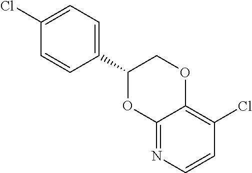 [SMILES: Clc1ccc([C@@H]2COc3c(Cl)ccnc3O2)cc1]

The mixture of (3R)-3-(4-chlorophenyl)-5-oxido-2,3-dihydro-[1,4]dioxino[2,3-b]pyridin-5-ium (145 mg, 550 μmol) in POCl₃ (422 mg, 2.75 mmol) was stirred at 90° C. for 1 h. The reaction mixture was concentrated. The residue was diluted with sat. aq. NaHCO₃ (20 mL) and extracted with EtOAc (20 mL×2). The organic layer was dried over Na₂SO₄, filtered and concentrated under reduced pressure to give the crude product (3R)-8-chloro-3-(4-chlorophenyl)-2,3-dihydro-[1,4]dioxino[2,3-b]pyridine (80 mg, 51.6% yield). LC-MS: m/z 282.0 (M+H)⁺.

### Step F tert-butyl (R)-4-(3-(4-chlorophenyl)-2,3-dihydro-[1,4]dioxino[2,3-b]pyridin-8-yl)-3,6-dihydropyridine-1(2H)-carboxylate

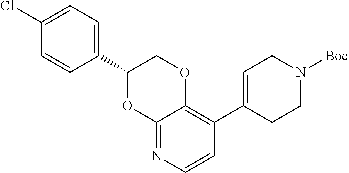 [SMILES: CC(C)(C)OC(=O)N1CC=C(c2ccnc3c2OC[C@@H](c2ccc(Cl)cc2)O3)CC1]

To a solution of (3R)-8-chloro-3-(4-chlorophenyl)-2,3-dihydro-[1,4]dioxino[2,3-b]pyridine (230 mg, 815 μmol) and tert-butyl 4-(4,4,5,5-tetramethyl-1,3,2-dioxaborolan-2-yl)-3,6-dihydropyridine-1(2H)-carboxylate (252 mg, 815 μmol) in THF (5 mL) and H₂O (3 mL) was added XPhos Pd G3 (69.0 mg, 81.5 μmol) and Cs₂CO₃ (531 mg, 1.63 mmol). The reaction mixture was stirred at 80° C. for 16 h. After cooling, the reaction mixture was diluted with brine and extracted with EtOAc (50 mL×2). The organic layer was dried over Na₂SO₄, filtered and concentrated. The residue was purified by flash silica gel chromatography (Silica Flash Column, Eluent of 10-20% EtOAc/PE gradient) to give tert-butyl (R)-4-(3-(4-chlorophenyl)-2,3-dihydro-[1,4]dioxino[2,3-b]pyridin-8-yl)-3,6-dihydropyridine-1(2H)-carboxylate (200 mg, 57.2% yield). LC-MS: m/z 429.2 (M+H)⁺.

### Step G tert-butyl (R)-4-(3-(4-chlorophenyl)-2,3-dihydro-[1,4]dioxino[2,3-b]pyridin-8-yl)piperidine-1-carboxylate

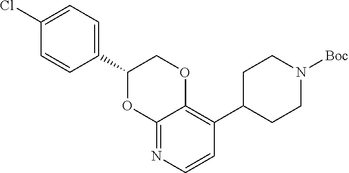 [SMILES: CC(C)(C)OC(=O)N1CCC(c2ccnc3c2OC[C@@H](c2ccc(Cl)cc2)O3)CC1]

To a solution of tert-butyl 4-[(3R)-3-(4-chlorophenyl)-2,3-dihydro-[1,4]dioxino[2,3-b]pyridin-8-yl]-3,6-dihydro-2H-pyridine-1-carboxylate (200 mg, 466 μmol) in EtOAc (5 mL) was added PtO₂ (318 mg, 1.40 mmol) under N₂ atmosphere. The suspension was degassed and purged with H₂ for 3 times. The mixture was stirred under H₂ (35 Psi) at 25° C. for 16 h. After filtration, the filtrate was concentrated to give tert-butyl (R)-4-(3-(4-chlorophenyl)-2,3-dihydro-[1,4]dioxino[2,3-b]pyridin-8-yl)piperidine-1-carboxylate (200 mg, 99% yield). LC-MS: m/z 431.2 (M+H)⁺.

### Step H (R)-3-(4-chlorophenyl)-8-(piperidin-4-yl)-2,3-dihydro-[1,4]dioxino[2,3-b]pyridine TFA salt

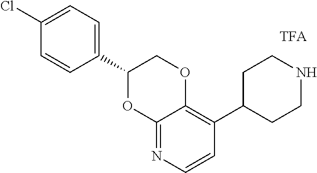 [SMILES: Clc1ccc([C@@H]2COc3c(C4CCNCC4)ccnc3O2)cc1]

To a solution of tert-butyl (R)-4-(3-(4-chlorophenyl)-2,3-dihydro-[1,4]dioxino[2,3-b]pyridin-8-yl)piperidine-1-carboxylate (200 mg, 464 μmol) in DCM (2 mL) was added TFA (2 mL). The mixture was stirred at 25° C. for 2 h. The solvent was removed to give (3R)-3-(4-chlorophenyl)-8-(4-piperidyl)-2,3-dihydro-[1,4]dioxino[2,3-b]pyridine TFA salt (150 mg, crude). LC-MS: m/z 331.2 (M+H)⁺.

### Step I 2-((4-((R)-3-(4-chlorophenyl)-2,3-dihydro-[1,4]dioxino[2,3-b]pyridin-8-yl)piperidin-1-yl)methyl)-1-(((S)-oxetan-2-yl)methyl)-1H-benzo[d]imidazole-6-carboxylic acid

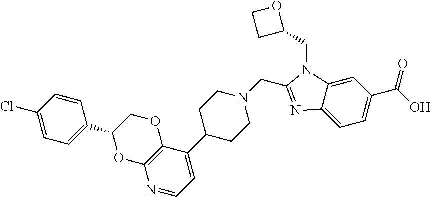 [SMILES: O=C(O)c1ccc2nc(CN3CCC(c4ccnc5c4OC[C@@H](c4ccc(Cl)cc4)O5)CC3)n(C[C@@H]3CCO3)c2c1]

2-((4-((R)-3-(4-chlorophenyl)-2,3-dihydro-[1,4]dioxino[2,3-b]pyridin-8-yl)piperidin-1-yl)methyl)-1-(((S)-oxetan-2-yl)methyl)-1H-benzo[d]imidazole-6-carboxylic acid (Compound 130) was synthesized according to the procedures described for the preparation of Example A5 (step A to B) by using (R)-3-(4-chlorophenyl)-8-(piperidin-4-yl)-2,3-dihydro-[1,4]dioxino[2,3-b]pyridine TFA salt and methyl (S)-2-(chloromethyl)-1-(oxetan-2-ylmethyl)-1H-benzo[d]imidazole-6-carboxylate in step A. LC-MS: m/z 575.3 (M+H)⁺. ¹H NMR (400 MHz, DMSO-d6) δ 8.28 (s, 1H), 8.20 (s, 1H), 7.81 (d, J=8.4 Hz, 1H), 7.74 (d, J=5.0 Hz, 1H), 7.64 (d, J=8.6 Hz, 1H), 7.52 (s, 3H), 6.94 (d, J=5.2 Hz, 1H), 5.46 (d, J=7.8 Hz, 1H), 5.10 (d, J=6.8 Hz, 1H), 4.80 (dd, J₁=15.2 Hz, J₂=7.2 Hz, 1H), 4.66 (d, J=14.4 Hz, 1H), 4.52 (d, J=9.4 Hz, 2H), 4.42-4.36 (m, 1H), 4.09 (d, J=10.8 Hz, 1H), 3.95 (d, J=13.4 Hz, 2H), 3.80 (d, J=13.6 Hz, 1H), 3.03 (s, 1H), 2.89 (d, J=8.6 Hz, 2H), 2.77-2.71 (m, 1H), 2.47-2.42 (m, 1H), 2.29-2.16 (m, 2H), 1.80-1.61 (m, 4H).

Example compound 128 was synthesized using a similar procedure described in the Example A11 above using the appropriate materials.

### Example A12

### 2-[[4-[(2R)-2-(4-chlorophenyl)-6-fluoro-2,3-dihydro-1,4-benzodioxin-5-yl]-1-piperidyl]methyl]-3-[[(2S)-oxetan-2-yl]methyl]benzimidazole-5-carboxylic acid (Compound 129)

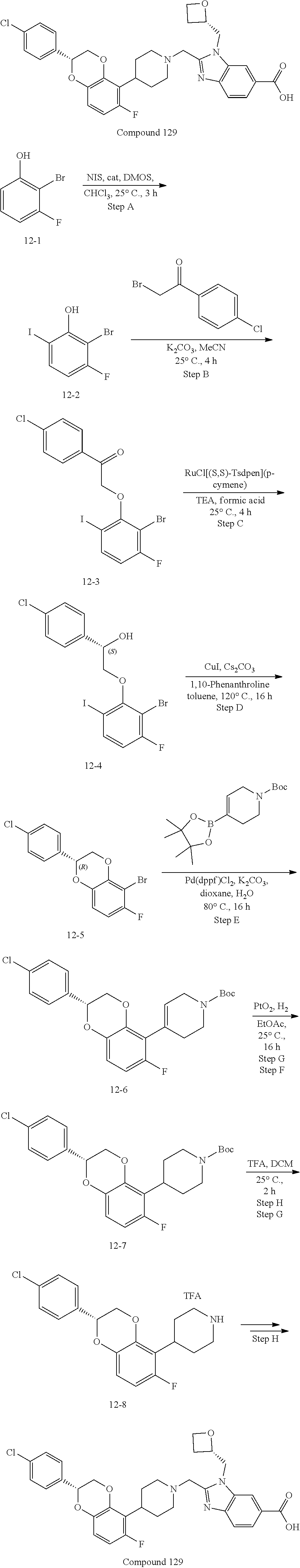

### Step A 2-bromo-3-fluoro-6-iodophenol

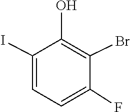

To a solution of 2-bromo-3-fluoro-phenol (9.42 g, 50 mmol) in CHCl₃ (30 mL) was added NIS (10 g, 44.4 mmol) and DMSO (771 mg, 9.86 mmol). The resulting mixture was stirred at 25° C. for 3 h. The mixture was diluted with brine, extracted with EtOAc (100 mL×2). The combined organic layer was washed with sat. aq. Na₂SO₃ (50 mL), dried over Na₂SO₄, filtered and concentrated. The residue was purified by flash silica gel chromatography (Silica Flash Column, Eluent of 10~30% EtOAc/PE gradient) to give 2-bromo-3-fluoro-6-iodo-phenol (3.8 g, 24% yield).

### Step B 2-(2-bromo-3-fluoro-6-iodophenoxy)-1-(4-chlorophenyl)ethan-1-one

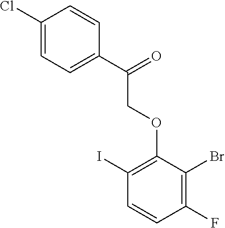 [SMILES: O=C(COc1c(I)ccc(F)c1Br)c1ccc(Cl)cc1]

To a solution of 2-bromo-3-fluoro-6-iodo-phenol (3.8 g, 12 mmol) and 2-bromo-1-(4-chlorophenyl)ethanone (2.80 g, 12 mmol) in MeCN (20 mL) was added K₂CO₃ (3.31 g, 24.0 mmol). The mixture was stirred at 25° C. for 4 h. The reaction mixture was diluted with H₂O (50 mL) and extracted with EtOAc (50 mL×2). The combined organic phase was dried over Na₂SO₄, filtered and concentrated. The residue was purified by flash silica gel chromatography (Silica Flash Column, Eluent of 10~20% EtOAc/PE gradient) to give 2-(2-bromo-3-fluoro-6-iodo-phenoxy)-1-(4-chlorophenyl)ethanone (3 g, 53.3% yield).

### Step C (S)-2-(2-bromo-3-fluoro-6-iodophenoxy)-1-(4-chlorophenyl)ethan-1-ol

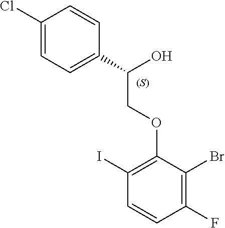

To a solution of formic acid (15 mL) and TEA (75 mL) was added RuCl[(S,S)-Tsdpen](p-cymene) (407 mg, 639 μmol). The mixture was stirred at 25° C. for 1 h, then 2-(2-bromo-3-fluoro-6-iodo-phenoxy)-1-(4-chlorophenyl)ethanone (6 g, 12.8 mmol) was added. The resulting mixture was stirred at 25° C. for 4 h. The reaction mixture was diluted with water, extracted with EtOAc (50 mL×2). The combined organic layer was dried over Na₂SO₄, filtered and concentrated. The residue was purified by flash silica gel chromatography (Silica Flash Column, Eluent of 10-40% EtOAc/PE gradient) to give (S)-2-(2-bromo-3-fluoro-6-iodophenoxy)-1-(4-chlorophenyl)ethan-1-ol (2.5 g, 5.30 mmol). ¹H NMR (400 MHz, CDCl₃) δ 7.72 (dd, J₁=8.8 Hz, J₂=6.0 Hz, 1H), 7.40-7.47 (m, 2H), 7.38-7.33 (m, 2H), 6.77 (t, J=8.4 Hz, 1H), 5.28 (d, J=8.8 Hz, 1H), 4.21-4.12 (m, 1H), 4.12-4.02 (m, 1H), 3.14 (d, J=2.0 Hz, 1H).

### Step D (R)-5-bromo-2-(4-chlorophenyl)-6-fluoro-2,3-dihydrobenzo[b][1,4]dioxine

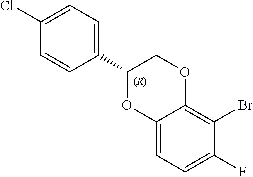

To a solution of (1 S)-2-(2-bromo-3-fluoro-6-iodo-phenoxy)-1-(4-chlorophenyl)ethanol (500 mg, 1.06 mmol) in toluene (5 mL) was added CuI (20.20 mg, 106.00 μmol), 1,10-phenanthroline (38.2 mg, 212 μmol) and Cs₂CO₃ (691 mg, 2.12 mmol). The reaction mixture was stirred at 120° C. for 16 h. After cooling, the reaction mixture was diluted with water, extracted with EtOAc (50 mL×2). The combined organic layer was dried over Na₂SO₄, filtered and concentrated. The residue was purified by flash silica gel chromatography (Silica Flash Column, Eluent of 10~20% EtOAc/PE gradient) to give (2R)-5-bromo-2-(4-chlorophenyl)-6-fluoro-2,3-dihydro-1,4-benzodioxine (250 mg, 68.7% yield). LC-MS: m/z 343.2 (M+H)⁺. ¹H NMR (400 MHz, CDCl₃) δ 7.45-7.39 (m, 2H), 7.38-7.33 (m, 2H), 6.94-6.87 (m, 1H), 6.76-6.64 (m, 1H), 5.06 (d, J=8.8 Hz, 1H), 4.49 (dd, J₁=11.6 Hz, J₂=1.6 Hz, 1H), 4.04 (dd, J₁=11.2, J₂=9.4 Hz, 1H)

### Step E tert-butyl (R)-4-(2-(4-chlorophenyl)-6-fluoro-2,3-dihydrobenzo[b][1,4]dioxin-5-yl)-3,6-dihydropyridine-1(2H)-carboxylate

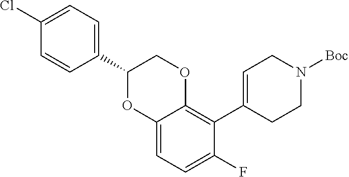 [SMILES: CC(C)(C)OC(=O)N1CC=C(c2c(F)ccc3c2OC[C@@H](c2ccc(Cl)cc2)O3)CC1]

To a solution of (2R)-5-bromo-2-(4-chlorophenyl)-6-fluoro-2,3-dihydro-1,4-benzodioxine (250 mg, 728 μmol) and tert-butyl 4-(4,4,5,5-tetramethyl-1,3,2-dioxaborolan-2-yl)-3,6-dihydropyridine-1(2H)-carboxylate (270 mg, 873 μmol) in dioxane (2 mL) and H₂O (0.2 mL) was added Pd(dppf)Cl₂ (53.2 mg, 72.8 μmol) and K₂CO₃ (201 mg, 1.46 mmol). The reaction mixture was stirred at 80° C. for 16 h. After cooling, the reaction mixture was diluted with water, extracted with EtOAc (50 mL×2). The combined organic layer was dried over Na₂SO₄, filtered and concentrated. The residue was purified by flash silica gel chromatography (Silica Flash Column, Eluent of 0~20% EtOAc/PE gradient) to give tert-butyl (R)-4-(2-(4-chlorophenyl)-6-fluoro-2,3-dihydrobenzo[b][1,4]dioxin-5-yl)-3,6-dihydropyridine-1(2H)-carboxylate (200 mg, 61.7% yield). [0460]¹H NMR (400 MHz, CDCl₃) δ 7.38-7.30 (m, 2H), 7.30-7.23 (m, 2H), 6.82 (dd, J₁=9.0 Hz, J₂=5.3 Hz, 1H), 6.62 (t, J=8.4 Hz, 1H), 4.97 (d, J=8.8 Hz, 1H), 4.40 (dd, J₁=11.6 Hz, J₂=1.2 Hz, 1H), 3.96 (dd, J₁=11.0, J₂=9.6 Hz, 1H)

### Step F tert-butyl (R)-4-(2-(4-chlorophenyl)-6-fluoro-2,3-dihydrobenzo[b][1,4]dioxin-5-yl)piperidine-1-carboxylate

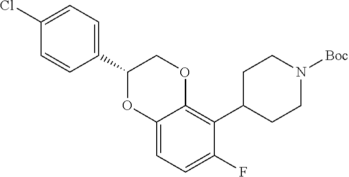 [SMILES: CC(C)(C)OC(=O)N1CCC(c2c(F)ccc3c2OC[C@@H](c2ccc(Cl)cc2)O3)CC1]

To a solution of tert-butyl 4-[(2R)-2-(4-chlorophenyl)-6-fluoro-2,3-dihydro-1,4-benzodioxin-5-yl]-3,6-dihydro-2H-pyridine-1-carboxylate (200 mg, 449 μmol) in EtOAc (3 mL) was added PtO₂ (102 mg, 449 μmol) under N₂ atmosphere. The suspension was degassed and purged with H₂ for 3 times. The mixture was stirred under H₂ (35 Psi) at 25° C. for 16 h. After filtration, the filtrate was concentrated to give tert-butyl 4-[(2R)-2-(4-chlorophenyl)-6-fluoro-2,3-dihydro-1,4-benzodioxin-5-yl]piperidine-1-carboxylate (100 mg, 50% yield). LC-MS: m/z 348.1 (M+H-Boc)⁺.

### Step G (R)-4-(2-(4-chlorophenyl)-6-fluoro-2,3-dihydrobenzo[b][1,4]dioxin-5-yl)piperidine TFA salt

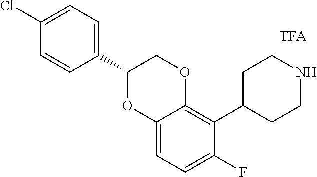 [SMILES: Fc1ccc2c(c1C1CCNCC1)OC[C@@H](c1ccc(Cl)cc1)O2]

To a solution of tert-butyl 4-[(2R)-2-(4-chlorophenyl)-6-fluoro-2,3-dihydro-1,4-benzodioxin-5-yl]piperidine-1-carboxylate (100 mg, 223 μmol) in DCM (2 mL) was added TFA (2 mL). The reaction mixture was stirred at 25° C. for 3 h. The mixture was concentrated to give 4-[(2R)-2-(4-chlorophenyl)-6-fluoro-2,3-dihydro-1,4-benzodioxin-5-yl]piperidine TFA salt (50 mg, crude), which used in the next step without purification. LC-MS: m/z 348.1 (M+H)⁺.

### Step H 2-((4-((R)-2-(4-chlorophenyl)-6-fluoro-2,3-dihydrobenzo[b][1,4]dioxin-5-yl)piperidin-1-yl)methyl)-1-(((S)-oxetan-2-yl)methyl)-1H-benzo[d]imidazole-6-carboxylic acid

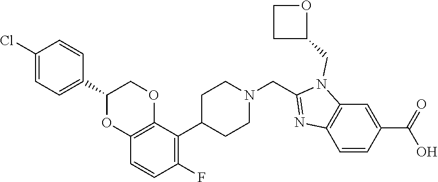 [SMILES: O=C(O)c1ccc2nc(CN3CCC(c4c(F)ccc5c4OC[C@@H](c4ccc(Cl)cc4)O5)CC3)n(C[C@@H]3CCO3)c2c1]

2-[[4-[(2R)-2-(4-chlorophenyl)-6-fluoro-2,3-dihydro-1,4-benzodioxin-5-yl]-1-piperidyl]methyl]-3-[[(2S)-oxetan-2-yl]methyl]benzimidazole-5-carboxylic acid (Compound 129) was synthesized according to the procedures described for the preparation of Example A5 (step A to B) by using (R)-4-(2-(4-chlorophenyl)-6-fluoro-2,3-dihydrobenzo[b][1,4]dioxin-5-yl)piperidine TFA salt and methyl (S)-2-(chloromethyl)-1-(oxetan-2-ylmethyl)-1H-benzo[d]imidazole-6-carboxylate in step A. LC-MS: m/z 592.2 (M+H)⁺. ¹H NMR (400 MHz, DMSO-d6) δ 8.27 (s, 1H), 7.80 (d, J=8.4 Hz, 1H), 7.63 (d, J=8.4 Hz, 1H), 7.51 (s, 4H), 6.91-6.80 (m, 1H), 6.75-6.61 (m, 1H), 5.23 (d, J=8.4 Hz, 1H), 5.12 (d, J=6.2 Hz, 1H), 4.78 (dd, J₁=14.8 Hz, J₂=7.2 Hz, 1H), 4.68-4.62 (m, 1H), 4.55-4.48 (m, 2H), 4.40 (d, J=7.6 Hz, 1H), 4.12-4.03 (m, 1H), 3.94 (d, J=12.8 Hz, 1H), 3.76 (d, J=13.6 Hz, 1H), 3.02 (d, J=10.3 Hz, 2H), 2.85 (d, J=9.7 Hz, 2H), 2.70 (d, J=9.4 Hz, 2H), 2.26-2.17 (m, 1H), 2.12-2.05 (m, 2H), 1.65-1.53 (m, 2H).

### Example A13

### 2-({4-[(2R)-2-[1-(2,2-difluoroethyl)-1H-indazol-6-yl]-2,3-dihydro-1,4-benzodioxin-5-yl]piperidin-1-yl}methyl)-1-{[(2S)-oxetan-2-yl]methyl}-1H-1,3-benzodiazole-6-carboxylic acid (Compound 102)

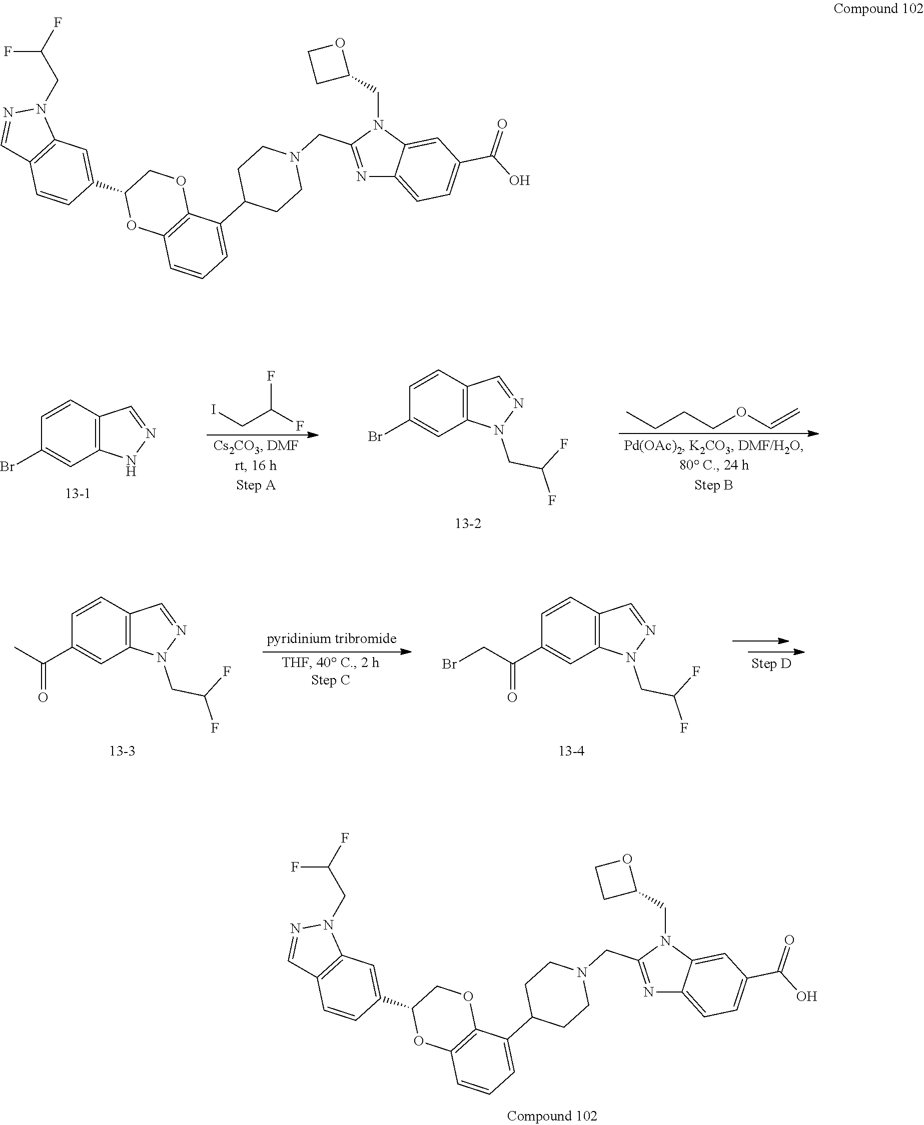

### Step A 6-bromo-1-(2,2-difluoroethyl)-1H-indazole

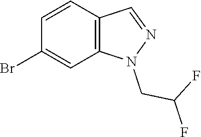 [SMILES: FC(F)Cn1ncc2ccc(Br)cc21]

To a solution of 6-bromo-1H-indazole (5.0 g, 25.38 mmol) in DMF (50 mL) was added 1,1-difluoro-2-iodoethane (4.9 g, 25.38 mmol) and Cs₂CO₃ (16.5 g, 50.75 mmol). The reaction was stirred at 25° C. for 16 h. After the reaction was completed, the reaction was diluted with H₂O (300 mL) and extracted with ethyl acetate (300 mL). The organic layer was washed with brine, dried over sodium sulfate, filtered and concentrated. The residue was purified by flash silica gel chromatography (Silica Flash Column, Eluent of 0~10% EtOAc/PE gradient) to give 6-bromo-1-(2,2-difluoroethyl)-1H-indazole (3. g, 57.6% yield). ¹H NMR (400 MHz, DMSO-d6) δ 8.20 (d, J=1.2 Hz, 1H), 8.12 (s, 1H), 7.76 (d, J=8.4 Hz, 1H), 7.33 (dd, J₁=8.4 Hz, J₂=1.2 Hz, 1H), 6.59-6.31 (m, 1H), 5.02-4.94 (m, 2H).

### Step B 1-(1-(2,2-difluoroethyl)-1H-indazol-6-yl)ethan-1-one

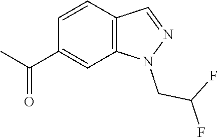 [SMILES: CC(=O)c1ccc2cnn(CC(F)F)c2c1]

To a solution of 6-bromo-1-(2,2-difluoroethyl)-1H-indazole (2.0 g, 7.66 mmol) in DMF/H₂O (16 mL/2 mL) was added 1-(vinyloxy)butane (2.3 g, 22.98 mmol), K₂CO₃ (4.2 g, 30.64 mmol), Pd(OAc)₂ (172 mg, 0.77 mmol) and 1,3-bis(diphenylphosphino)propane (632 mg, 1.53 mmol). The reaction mixture was stirred at 80° C. for 24 h under N₂ in the seal tube. After the reaction was completed, the reaction was filtered and concentrated, the residue was purified by flash silica gel chromatography (Silica Flash Column, Eluent of 0~15% EtOAc/PE gradient) to give 1-(1-(2,2-difluoroethyl)-1H-indazol-6-yl)ethan-1-one (660 mg, 38.4% yield). LC-MS: m/z 225.1 (M+H)⁺.

### Step C 2-bromo-1-(1-(2,2-difluoroethyl)-1H-indazol-6-yl)ethan-1-one

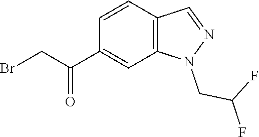 [SMILES: O=C(CBr)c1ccc2cnn(CC(F)F)c2c1]

To a solution of 1-(1-(2,2-difluoroethyl)-1H-indazol-6-yl)ethan-1-one (660 mg, 2.94 mmol) in THF (8 mL) was added pyridinium tribromide (942 mg, 2.94 mmol). The reaction was stirred at 40° C. for 2 h. After cooling, the reaction was quenched with H₂O (100 mL) and extracted with ethyl acetate (100 mL). The organic layer was washed with brine, dried over sodium sulfate, filtered and concentrated. The residue was purified by reverse ISCO (0-80% CH₃CN in water (0.1% NH₄HCO₃)) to give 2-bromo-1-(1-(2,2-difluoroethyl)-1H-indazol-6-yl)ethan-1-one (470 mg, 52.7% yield). LC-MS: m/z 302.9 (M+H)⁺.

### Step D 2-({4-[(2R)-2-[1-(2,2-difluoroethyl)-1H-indazol-6-yl]-2,3-dihydro-1,4-benzodioxin-5-yl]piperidin-1-yl}methyl)-1-{[(2S)-oxetan-2-yl]methyl}-1H-1,3-benzodiazole-6-carboxylic acid

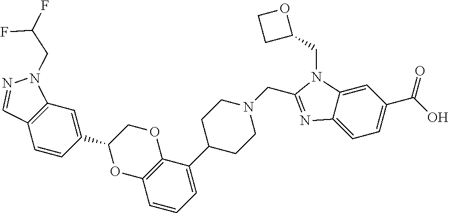 [SMILES: O=C(O)c1ccc2nc(CN3CCC(c4cccc5c4OC[C@@H](c4ccc6cnn(CC(F)F)c6c4)O5)CC3)n(C[C@@H]3CCO3)c2c1]

2-({4-[(2R)-2-[1-(2,2-difluoroethyl)-1H-indazol-6-yl]-2,3-dihydro-1,4-benzodioxin-5-yl]piperidin-1-yl}methyl)-1-{[(2S)-oxetan-2-yl]methyl}-1H-1,3-benzodiazole-6-carboxylic acid (Compound 102) was synthesized according to the procedures described for the preparation of Example A12 (step B to H) by using 2-bromo-1-(1-(2,2-difluoroethyl)-1H-indazol-6-yl)ethan-1-one and 2,6-dibromophenol in step B. LC-MS: m/z 644.4 (M+H)⁺. ¹H NMR (400 MHz, DMSO-d6) δ 8.27 (s, 1H), 8.19 (s, 1H), 7.88 (s, 1H), 7.85-7.78 (m, 2H), 7.64 (d, J=8.4 Hz, 1H), 7.31 (d, J=8.0 Hz, 1H), 6.89-6.78 (m, 3H), 6.43 (m, 1H), 5.38 (dd, J₁=8.4 Hz, J₂=2.0 Hz, 1H), 5.17-5.06 (m, 1H), 4.97-4.89 (m, 2H), 4.83 (dd, J₁=15.6 Hz, J₂=7.2 Hz, 1H), 4.68 (dd, J₁=14.8 Hz, J₂=2.4 Hz, 1H), 4.55-4.47 (m, 2H), 4.43-4.34 (m, 1H), 4.12 (dd, J₁=19.2 Hz, J₂=18.0 Hz, 1H), 3.96 (d, J=2.4 Hz, 1H), 3.82 (d, J=13.6 Hz, 1H), 3.79 (d, J=13.2 Hz, 1H), 3.06-2.97 (m, 1H), 2.94-2.81 (m, 2H), 2.75-2.65 (m, 1H), 2.47-2.40 (m, 1H), 2.35-2.18 (m, 2H), 1.85-1.55 (m, 4H). ¹⁹F NMR (377 MHz, DMSO-d6) δ −121.77.

The following molecules were synthesized using a similar procedure described in the Examples above using the appropriate starting material.

| Compound | |
|---|---|
| No. | LC-MS: m/z |
| 101 | 614.2 (M + H)⁺ |
| 102 | 644.4 (M + H)⁺ |
| 103 | 583.2 (M + H)⁺ |
| 104 | 583.2 (M + H)⁺ |
| 105 | 603.5 (M + H)⁺ |
| 106 | 579.4 (M + H)⁺ |
| 107 | 574.4 (M + H)⁺ |
| 108 | 601.3 (M + H)⁺ |
| 109 | 597.7 (M + H)⁺ |
| 110 | 577.7 (M + H)⁺ |
| 111 | 592.2 (M + H)⁺ |
| 112 | 606.2 (M + H)⁺ |
| 113 | 638.4 (M + H)⁺ |
| 114 | 595.4 (M + H)⁺ |
| 115 | 608.3 (M + H)⁺ |
| 116 | 626.1 (M + H)⁺ |
| 117 | 642.4 (M + H)⁺ |
| 118 | 595.1 (M + H)⁺ |
| 119 | 604.1 (M + H)⁺ |
| 120 | 644.3 (M + H)⁺ |
| 121 | 610.1 (M + H)⁺ |
| 122 | 632.2 (M + H)⁺ |
| 123 | 622.2 (M + H)⁺ |
| 124 | 625.2 (M + H)⁺ |
| 125 | 572.3 (M + H)⁺ |
| 126 | 586.1 (M + H)⁺ |
| 127 | 586.1 (M + H)⁺ |
| 128 | 575.6 (M + H)⁺ |
| 129 | 592.2 (M + H)⁺ |
| 130 | 575.3 (M + H)⁺ |

### BIOLOGICAL EXAMPLES

### Biological Example 1: cAMP Assays

Activation of GLP-1 receptor is known to stimulate cyclic AMP (cAMP) production in cells which indicates primary coupling to the G as subunit of the G protein heterotrimeric complex. Evidence suggests signaling through G as induced cAMP stimulation elicits the desired pharmacological response regarding insulin release from pancreatic β-cells.

To optimize functional activity directed toward G as coupling, a HEK293/CRE-Luc cell line developed by HDB stably expressing the GLP-1 Receptor was used. 200× concentration of compound working solutions were prepared (Agilent Technologies Bravo) with ½ log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550.1×10 5 cells/mL HEK293/GLP1R/CRE-LUC (HD Biosciences) cell suspensions prepared with assay buffer [DPBS containing 0.5 mM IBMX (Sigma, Cat #I5879) and 0.1% BSA (GENVIEW, Cat #FA016-100 g)], 10 μL cell suspensions were added to each well of previous generated assay plate which already contains 50 nL compound at 200× concentration using ThermoFisher Multidrop Combi (1000 cells/well). Seal the plate and incubate at 37° C. with 5% CO₂ for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

$$\% Activity = 100 \% \times ( mean RLU of test sample - mean RLU of vehicle control ) / ( mean RLU of MAX control - mean RLU of vehicle control )$$

Table 2 shows the biological activity of compounds in GLP-1R agonist cAMP stimulation assay (EC₅₀).

TABLE 2

| Compound | |
|---|---|
| No. | EC₅₀ (μM) |
| 101 | 0.000024 |
| 102 | 0.000065 |
| 103 | 0.000057 |
| 104 | 0.000027 |
| 105 | 0.000012 |
| 106 | 0.0016 |
| 107 | 0.00018 |
| 108 | 0.000025 |
| 109 | 0.000041 |
| 110 | 0.1 |
| 111 | 0.000017 |
| 112 | 0.000026 |
| 113 | 0.004 |
| 114 | 0.000024 |
| 115 | 0.000055 |
| 116 | 0.000029 |
| 117 | 0.00042 |
| 118 | 0.000011 |
| 119 | 0.000011 |
| 120 | 0.000005 |
| 121 | 0.000031 |
| 122 | 0.0000012 |
| 123 | 0.0019 |
| 124 | 0.00068 |
| 125 | 0.00077 |
| 126 | 0.0016 |
| 127 | 0.5 |
| 128 | 0.00079 |
| 129 | 0.00038 |
| 130 | 0.00016 |

What is claimed is:

1. A compound of Formula I: 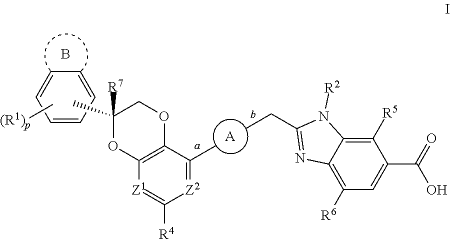
or a pharmaceutically acceptable salt, isotopically enriched analog, stereoisomer, mixture of stereoisomers, or prodrug thereof, wherein:
ring A is 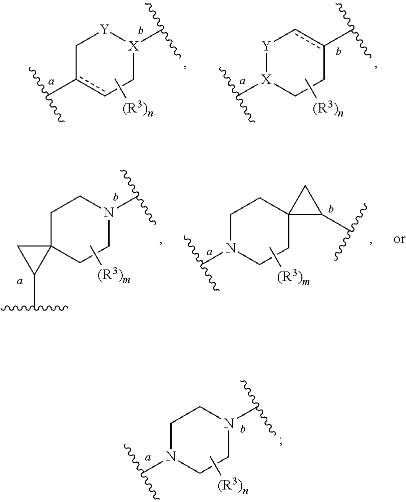
where [symbol] the bond represents a single or double bond;
where X is N or CH and Y is O, CH₂, CHR³, or C(R³)₂;
Z¹ is N or CR⁴;
Z² is N or CR⁴;
ring B is absent; or ring B is present and, together with the carbon atoms of the phenyl ring to which it is fused, forms a C₅₋₁₀ cycloalkyl, heterocyclyl, aryl, or heteroaryl; wherein ring B is independently optionally substituted with one to five R¹;
p is 0, 1, 2, or 3;
n is 0, 1, 2, or 3;
m is 0, 1, 2, or 3;
each R¹ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹OC(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₁₋₉ haloalkyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl is independently optionally substituted with one to five Z;
R² is hydrogen, heterocyclyl, C₁₋₆ alkyl, or C₁₋₆ alkyl substituted with C₁₋₆ alkoxy, C₁₋₆ thioalkoxy, C₁₋₆ haloalkoxy, S(O)₂(C₁₋₆ alkyl), C₃₋₆ cycloalkyl, 3- to 6-membered heterocyclyl, phenyl, or 5- to 6-membered heteroaryl; wherein each of the C₃₋₆ cycloalkyl, 3- to 6-membered heterocyclyl, phenyl, or 5- to 6-membered heteroaryl is optionally substituted with one to four R¹²;
R³ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀ cycloalkyl; or
two R³, together with the carbon atom to which they are attached, form a C₃₋₁₀ cycloalkyl or heterocyclyl; wherein each C₃₋₁₀cycloalkyl or heterocyclyl is independently optionally substituted with one or two halo;
each R⁴ is independently hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁵ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁶ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
R⁷ is hydrogen, cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀cycloalkyl, heterocyclyl, aryl, or heteroaryl is independently optionally substituted with one to five Zia;
each R¹² is independently selected from the group consisting of cyano, halo, hydroxy, SH, nitro, C₁₋₆ alkyl, C₂₋₆ alkenyl, C₂₋₆ alkynyl, C₁₋₆ haloalkyl, C₁₋₆ cyanoalkyl, C₁₋₆ hydroxyalkyl, C₁₋₆ alkoxy, C₁₋₆ haloalkoxy, C₃₋₆ cycloalkyl, amino, C₁₋₆ alkylamino, and di-C₆ alkylamino;
each Z is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁ g alkyl, C₂₋₉ alkenyl, C₂₋₉ alkynyl, C₃₋₁₀ cycloalkyl, heterocyclyl, aryl, or heteroaryl is independently optionally substituted with one to five Zia;
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁, alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆cycloalkyl, heterocyclyl, aryl, or heteroaryl; wherein each —NH—C₁₋₉ alkyl, —N(C₁₋₉ alkyl)₂, —S—C₁₋₉ alkyl, C₁₋₉ alkoxy, C₁₋₉ alkyl, C₂₋₉ alkenyl, C₂₋₉ alkynyl, C₃₋₆ cycloalkyl, heterocyclyl, aryl, or heteroaryl of Zia is independently optionally substituted with one to five substituents selected from C₁₋₉ alkyl, oxo, halo, hydroxy, and cyano;
provided that any one or more of the following occurs:
A) ring A is 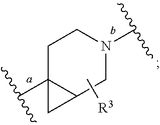
B) one of R⁵ and R⁶ is other than hydrogen;
C) R² is heterocyclyl optionally substituted with one to four R¹²; or
D) at least one R¹² is —C(O)—C₁₋₆ alkyl.

2. The compound of claim 1, wherein ring A is:

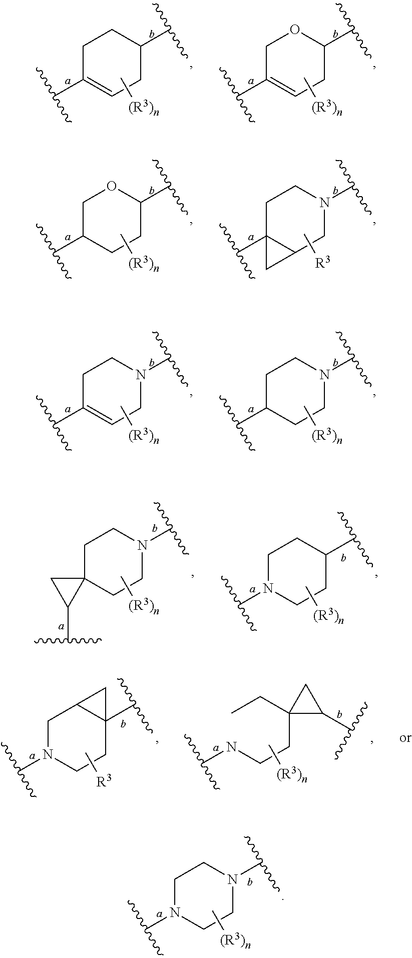

3. The compound of claim 1, wherein the compound is represented by Formula IA: 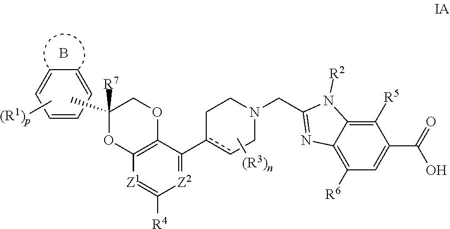
or a pharmaceutically acceptable salt, isotopically enriched analog, stereoisomer, mixture of stereoisomers, or prodrug thereof.

4. The compound of claim 1, wherein the compound is represented by Formula IB: 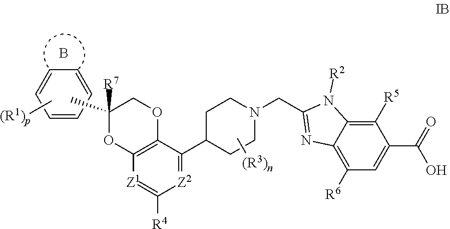
or a pharmaceutically acceptable salt, isotopically enriched analog, stereoisomer, mixture of stereoisomers, or prodrug thereof.

5. The compound of claim 1, wherein the compound is represented by Formula IC: 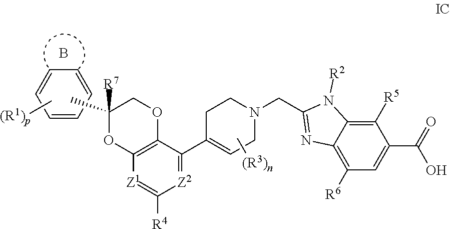
or a pharmaceutically acceptable salt, isotopically enriched analog, stereoisomer, mixture of stereoisomers, or prodrug thereof.

6. The compound of claim 1, wherein the compound is represented by Formula ID: 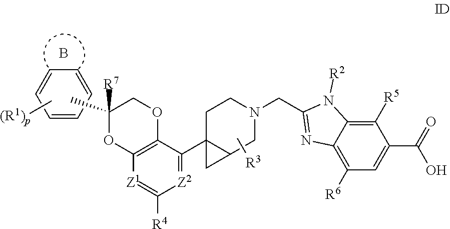
or a pharmaceutically acceptable salt, isotopically enriched analog, stereoisomer, mixture of stereoisomers, or prodrug thereof.

7. The compound of claim 1, wherein the compound is represented by Formula IE: 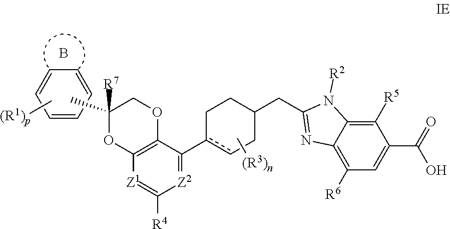
or a pharmaceutically acceptable salt, isotopically enriched analog, stereoisomer, mixture of stereoisomers, or prodrug thereof.

8. The compound of any preceding claim, wherein R⁵ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀cycloalkyl.

9. The compound of any preceding claim, wherein R⁶ is cyano, halo, hydroxy, C₁₋₉ alkyl, C₂₋₉ alkenyl, C₂₋₉ alkynyl, C₁₋₉ alkoxy, C₁₋₉ haloalkyl, C₁₋₉ haloalkoxy, or C₃₋₁₀cycloalkyl.

10. The compound of any preceding claim, wherein the moiety

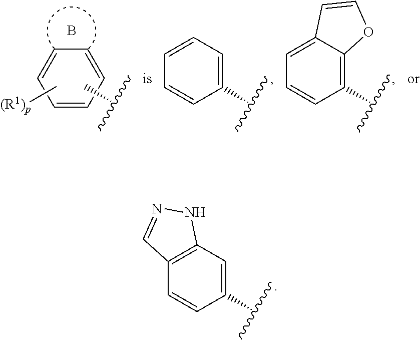

11. The compound of any preceding claim, wherein ring B is present.

12. The compound of any preceding claim, wherein the moiety

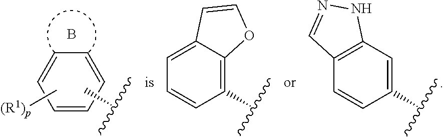

13. The compound of any preceding claim, wherein each R¹ is independently halo, cyano, or C₁₋₉ haloalkyl.

14. The compound of any preceding claim, wherein p is 1.

15. The compound of any preceding claim, wherein at least one R⁴ is other than hydrogen.

16. The compound of any preceding claim, wherein Z² is CR⁴; and R⁴ of Z² is cyano, halo, hydroxy, C₁₋₉ alkyl, C₂₋₉ alkenyl, C₂₋₉ alkynyl, C₁₋₆ cyanoalkyl, C₁₋₆ hydroxyalkyl, C₁₋₉ alkoxy, C₁₋₉ haloalkyl, C₁₋₉ haloalkoxy, or C₃₋₁₀ cycloalkyl.

17. The compound of any preceding claim, wherein R² is heterocyclyl or C₁₋₆ (alkyl substituted with C₃₋₆ cycloalkyl, 3- to 6-membered heterocyclyl, or 5- to 6-membered heteroaryl; wherein each of the heterocyclyl, C₃₋₆ cycloalkyl, 3- to 6-membered heterocyclyl, or 5- to 6-membered heteroaryl is optionally substituted with one to four, or one to three, or one to two, or one R¹².

18. The compound of any preceding claim, wherein R² is heterocyclyl or C₁₋₆ alkyl substituted with C₃₋₆cycloalkyl, 3- to 6-membered heterocyclyl, or 5- to 6-membered heteroaryl; wherein each of the heterocyclyl, C₃₋₆ cycloalkyl, 3- to 6-membered heterocyclyl, or 5- to 6-membered heteroaryl is optionally substituted with one to four, or one to three, or one to two, or one, cyano, halo, C₁₋₆ alkyl, C₁₋₆ cyanoalkyl, or —C(O)—C₁₋₆ alkyl.

19. The compound of any preceding claim, wherein R² is heterocyclyl optionally substituted with one to five substituents independently selected from halo, cyano, hydroxy, C₁₋₆ alkyl, C₁₋₆ haloalkyl, C₁₋₆ cyanoalkyl, C₁₋₆ hydroxyalkyl, C₁₋₆ alkoxy, C₁₋₆ haloalkoxy, and C₃₋₆ cycloalkyl.

20. The compound of any preceding claim, wherein R² is 3-6 membered heterocyclyl substituted with C₁₋₆ alkoxy.

21. The compound of any preceding claim, wherein each R⁴ is independently hydrogen or halo.

22. The compound of any preceding claim, wherein R¹ is hydrogen or halo.

23. The compound of any preceding claim, wherein R⁶ is hydrogen, halo, or C₁₋₉ alkoxy.

24. The compound of any preceding claim, wherein R¹ is hydrogen or halo; and R⁶ is halo or C₁₋₉ alkoxy.

25. The compound of any one of claims 1-23, wherein R⁵ is halo; and R⁶ is hydrogen, halo, or C₁₋₉ alkoxy.

26. The compound of any preceding claim, wherein R⁷ is hydrogen.

27. The compound of any preceding claim, wherein Z¹ is N.

28. The compound of any one of claims 1-15 and 17-27, wherein Z² is N.

29. The compound of any preceding claim, wherein the moiety 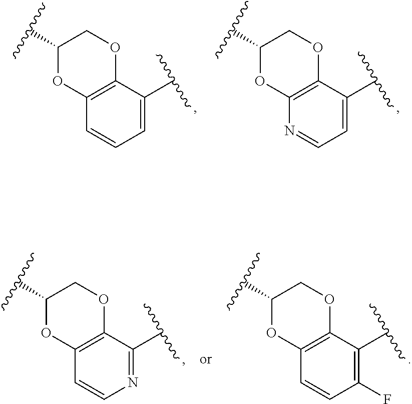 [SMILES: *c1cccc2c1OC[C@@H](*)O2 ; *c1ccnc2c1OC[C@@H](*)O2 ; *c1nccc2c1OC[C@@H](*)O2 ; *c1c(F)ccc2c1OC[C@@H](*)O2]
is

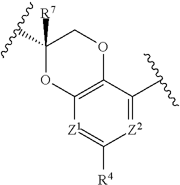

30. A compound selected from Table 1, or a pharmaceutically acceptable salt, stereoisomer, mixture of stereoisomers, or prodrug thereof.

31. A pharmaceutical composition comprising a compound of any preceding claim, or a pharmaceutically acceptable salt, stereoisomer, mixture of stereoisomers, or prodrug thereof, and a pharmaceutically acceptable excipient.

32. A method for treating a GLP-1 associated disease, disorder, or condition, the method comprising administering to a patient in need thereof an effective amount of a compound of any one of claims 1-30, or a pharmaceutically acceptable salt, stereoisomer, mixture of stereoisomers, or prodrug thereof, or the pharmaceutical composition according to claim 31.

33. The method of claim 32, wherein the disease, disorder, or condition is selected from the group consisting of type 1 diabetes mellitus, type 2 diabetes mellitus, early onset type 2 diabetes mellitus, idiopathic type 1 diabetes mellitus (Type 1 b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

34. A method of treating type 2 diabetes mellitus in a patient in need thereof, the method comprising administering to a patient in need thereof an effective amount of a compound of any one of claims 1-30, or a pharmaceutically acceptable salt, stereoisomer, mixture of stereoisomers, or prodrug thereof, or the pharmaceutical composition according to claim 31.

35. A method for modulating insulin levels in a patient in need of such modulating, the method comprising administering to a patient in need thereof an effective amount of a compound of any one of claims 1-30, or a pharmaceutically acceptable salt, stereoisomer, mixture of stereoisomers, or prodrug thereof, or the pharmaceutical composition according to claim 31.

36. A method for modulating glucose levels in a patient in need of such modulating, the method comprising administering to a patient in need thereof an effective amount of a compound of any one of claims 1-30, or a pharmaceutically acceptable salt, stereoisomer, mixture of stereoisomers, or prodrug thereof, or the pharmaceutical composition according to claim 31.

37. The method of any one of claims 32-36, further comprising administering an additional therapy or therapeutic agent to the patient.

38. The method of claim 37, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

39. A process for preparing the compound of Formula I as in claim 1, or a pharmaceutically acceptable salt, isotopically enriched analog, stereoisomer, mixture of stereoisomers, or prodrug thereof, comprising contacting a compound of Formula I-1: 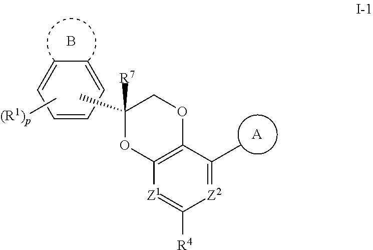
with a compound of Formula I-2: 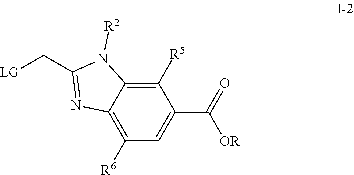
wherein LG is a leaving group; and R is hydrogen or C₁₋₆ alkyl optionally substituted with phenyl, under conditions sufficient to provide the compound of Formula I, or a pharmaceutically acceptable salt, isotopically enriched analog, stereoisomer, mixture of stereoisomers, or prodrug thereof.

40. The process of claim 39, wherein the process further comprises a hydrolysis or transesterification step prior to or after the contacting.
